(19) United States
(12) Patent Application Publication
(10) Pub. No.: US 2024/0175881 A1
(43) Pub. Date: May 30, 2024

(54) SIMPLIFIED METHODS FOR MEASURING LIVER FUNCTION

(71) Applicant: HepQuant, LLC, Denver, CO (US)

(72) Inventors: Michael P. McRae, Houston, TX (US); Steve Mark Helmke, Denver, CO (US); Gregory Thomas Everson, Englewood, CO (US)

(21) Appl. No.: 18/500,800

(22) Filed: Nov. 2, 2023

Related U.S. Application Data
(60) Provisional application No. 63/422,337, filed on Nov. 3, 2022.
(60) Provisional application No. 63/505,880, filed on Jun. 2, 2023.
(60) Provisional application No. 63/581,496, filed on Sep. 8, 2023.

(51) Int. Cl. G01N 33/74; G16H 50/20

(52) U.S. Cl. CPC G01N 33/74; G16H 50/20

(57) ABSTRACT

Simplified methods are provided for determining liver function based on a Compartmental Model (CM) method for analysis of various hepatic indices including cholate SHUNT test which closely approximates and elucidates the underlying physiology of hepatic uptake of cholate from systemic and portal circulations simultaneously.

### CROSS-REFERENCES TO RELATED APPLICATIONS

This application is being filed on Nov. 2, 2023, as a PCT International Patent Application that claims the benefit of priority to U.S. Provisional Application Ser. No. 63/422,337, filed Nov. 3, 2022, U.S. Provisional Application Ser. No. 63/505,880, filed Jun. 2, 2023, and U.S. Provisional Application Ser. No. 63/581,496, filed Sep. 8, 2023, each of which is incorporated by reference herein in its entirety.

### BACKGROUND OF THE INVENTION

Chronic liver disease (CLD) affects millions worldwide. Estimates suggest that as many as 50 million Americans have CLD, with over 15 million with fibrosis, and over 4 million with cirrhosis. The common etiologies of CLD include alcoholic liver disease, chronic viral hepatitis B or C, autoimmune hepatitis, primary biliary cholangitis, primary sclerosing cholangitis, and genetic conditions. But, as a result of the obesity pandemic, the most common emerging and expanding etiology for CLD is metabolic dysfunction-associated steatotic liver disease (MASLD), formerly known as nonalcoholic fatty liver disease (NAFLD), or metabolic dysfunction-associated steatohepatitis (MASH), formerly known as nonalcoholic steatohepatitis (NASH). The cholate SHUNT liver function test quantifies liver health (DSI, disease severity index) to aid providers, patients, and payers in the management of CLD patients.

The cholate SHUNT test quantifies liver function from portal and systemic clearances simultaneously using stable nonradioactive isotopes of carbon-13-labeled cholate (13C-CA) intravenously and deuterium-labeled cholate (d4-CA) orally. The test is minimally invasive, well tolerated, and only requires the sampling of peripheral venous blood. A disease severity index (DSI) score, a single score of liver health ranging from 0 (healthy) to 50 (severely impaired), has demonstrated reliability in reproducibility studies (Burton J R, Helmke S, Lauriski S, Kittelson J, Everson G T. The within-individual reproducibility of the disease severity index from the HepQuant SHUNT test of liver function and physiology. Transl Res. 2021; 233:5-15) and has detected early stages of disease, quantified disease severity, monitored disease progression, and measured treatment effects (Helmke S, Colmenero J, Everson G T. Noninvasive assessment of liver function. Curr Opin Gastroenterol. 2015:31). The test uniquely enables pharmaceutical companies to measure efficacy of their therapeutics by measuring improvements in liver function rather than waiting to clinically observe progressive liver failure. A pivotal study has been recently completed linking SHUNT test results to the likelihood of large esophageal varices, a precursor to variceal hemorrhage and the most lethal complication of CLD (ClinicalTrials.gov. The SHUNT-V Study for Varices NCT03583996).

The cholate SHUNT test of liver function and physiology can be useful for monitoring treatment effects and predicting risk for clinical outcome. The HepQuant cholate SHUNT test quantifies hepatic functional impairment from the simultaneous clearance of cholate from the systemic and portal circulations for the purpose of monitoring treatment effects or for predicting risk for clinical outcome. Test parameters, derived from a noncompartmental minimal model (MM), are reproducible and reliable (Translational Research 2021).

U.S. Pat. No. 8,613,904, Everson et al., discloses cholate SHUNT test methods for evaluating liver function in a patient comprising obtaining patient serum samples following administration of two distinguishable stable isotope labeled cholate compounds, laborious sample processing and analysis of patient serum samples utilizing GC-MS.

U.S. Pat. No. 8,778,299, Everson, discloses cholate SHUNT test methods for evaluating liver function comprising obtaining patient serum samples following administration of two distinguishable stable isotope labeled cholate compounds, processing and analysis of patient serum samples utilizing HPLC-MS. Methods for determining portal hepatic filtration rate (portal HFR, FLOW) from oral administration of a distinguishable agent are also provided.

U.S. Pat. No. 9,091,701, Everson, discloses methods for determining liver function and obtaining a Disease Severity Index (DSI) value in a patient comprising obtaining patient serum samples following administration of two distinguishable stable isotope labeled cholate compounds, processing and analysis of patient serum samples utilizing HPLC-MS.

U.S. Pat. No. 8,961,925, Everson, discloses methods for performing the STAT test comprising oral administration of a distinguishable agent and measuring the distinguishable compound in a single blood or serum sample following using HLPC-MS. Methods of estimating portal hepatic filtration rate (portal HFR) from STAT values are also provided.

US Pat. Appl. Pub. No. US 2021/0318274 A1, Everson et al., discloses improved methods for blood or serum sample preparation, analyte detection and quantification which may be applied to one or more of the SHUNT, FLOW, STAT, and DSI liver function tests.

The cholate SHUNT liver function test measures portal and systemic clearances simultaneously, comprising: an intravenous (IV) dose of carbon-13-labeled cholate (13C-CA), a simultaneous oral dose of a deuterium-labeled cholate (d4-CA), and 5 peripheral venous blood draws over 90 minutes.

Simplified test methods providing reliable measurements of liver function that significantly reduce time and invasiveness of the test are desirable.

### SUMMARY OF THE INVENTION

Simplified liver function test methods are provided that assess both liver function and physiology using naturally occurring endogenous cholates labeled with stable isotopes to probe hepatocyte uptake and hepatic perfusion. Results deliver accurate information about hepatic impairment in persons at risk/who have liver disease, monitoring treatment effects, and determining risk for clinical complications.

The present disclosure provides a series of second-generation liver function tests which provide accurate measures of liver function that correlate with first generation liver function tests (STAT, FLOW, and SHUNT tests), but which are significantly simplified in terms of the test administration.

Liver function test parameters (DSI, HFR, SHUNT, HR, etc.) require accurate and reliable measurements of the area under the oral and IV distinguishable cholate clearance curves (AUC_{Oral}, AUC_{IV}). The SHUNT test assesses both hepatocyte function and the portal circulation, making it a comprehensive diagnostic tool that addresses the pathogenesis of all types of CLD. However, the SHUNT test (SHUNT V1.0) requires both oral and intravenous (IV) cholate doses and six peripheral venous blood samples taken over 90 minutes. Potential sources of error related to the IV injection and multiple timed blood samples include poor IV access (difficulty in locating vein or catheter dislodges from vein), extravasation, errors in the timing of sampling, and variability of skill in administering the test.

Simplified liver function tests are provided herein including cholate DuO and SHUNT V2.0 liver function tests that reduce the number of blood samples by two-thirds and test time by one-third relative to SHUNT V1.0, provide reproducible measurements of liver function and physiology, and have potential to improve prediction of clinical outcome, enhance patient monitoring, and inform decisions about costly drugs, tests, and procedures.

The present disclosure provides simplified DuO and TRIO liver function tests, requiring only a limited number of datapoints (e.g., only 2 oral data points for DuO, no more than 2 oral data points for DuO, or 2 oral plus 1 or 2 IV data points for TRIO), as well as specialized analysis techniques to fit the systemic and portal clearance curves therefrom. Analysis methodologies are provided herein for each test.

The disclosure provides a method for assessing liver function in a subject having or suspected of having or contracting a liver disease, comprising obtaining blood or serum sample concentration data of an orally administered distinguishable cholate compound collected from a subject at two time points after oral administration; measuring the area under the curve of the blood or serum concentrations of the orally administered distinguishable cholate compound (AUCoral) in the subject comprising simulating a full oral clearance curve using a compartmental model of oral cholate clearance, the compartmental model comprising the oral distinguishable cholate compound concentration data at the two time points, body mass index (BMI), body weight (BW), and hematocrit (Hct) input values in the subject, and calculating the area comprising trapezoidal numerical integration to obtain the AUCoral; and calculating one or more indices of hepatic disease in the subject using the AUCoral, wherein the one or more indices is associated with liver function in the subject.

The method of the obtaining data at first and second time points after oral administration of distinguishable cholate may include receiving first and second blood or serum samples that had been collected from the subject at first and second time points following a single oral dose of a first distinguishable cholate compound; and analyzing the samples to obtain the oral concentration data at the first and second time points, optionally wherein the blood or serum samples had been collected within about 180 minutes, 120 minutes, 90 minutes, or within about 75 minutes, after the oral administration. In some cases, the first and second blood or serum samples had been collected from the subject between at least about 5 min to about 75 min, 10 min to 70 min, 20 min to 60 min, 25 min to 55 min, 30 min to 50 min, 35 to 45 min, or about 40 min apart. In some cases, the first and second blood or serum samples had been collected from the subject at about 10-30 min and about 40-80 min following the oral administration, respectively. In some cases, the first and second blood or serum samples had been collected from the subject at about 15-25 min and about 50-70 min following the oral administration, respectively. In some cases, the first and second blood or serum samples had been collected from the subject at about 20 min and about 60 min following the oral administration, respectively.

The method of obtaining data at first and second time points after oral administration of distinguishable cholate may include receiving a single blood or serum sample that had been collected from the subject following administration of a first oral dose of a first distinguishable cholate compound and a second oral dose of a second distinguishable cholate compound to the subject. The method may include analyzing the single sample to obtain the oral concentration data of the first distinguishable cholate compound and second distinguishable cholate compound at the two time points. The single blood or serum sample had been collected from the subject within about 180 minutes, 120 minutes, 90 minutes, or within about 75 minutes, after the first oral dose administration. In some cases, the first and second oral doses had been administered to the subject between at least about 5 min to about 75 min, 10 min to 70 min, 20 min to 60 min, 25 min to 55 min, 30 min to 50 min, 35 to 45 min, or about 40 min apart. In some cases, the single sample had been collected from the subject at about 10-30 min after the second oral dose and simultaneously at about 40-80 min after the first oral dose, to obtain the data at the first and second time points. In some cases, the single sample had been collected from the subject at about 15-25 min after the second oral dose and simultaneously at about 55-65 min after the first oral dose, to obtain the data at the first and second time points. In some cases, the single sample had been collected from the subject at about 20 min after the second oral dose and simultaneously at about 60 min after the first oral dose, to obtain the data at the first and second time points.

In some embodiments, the method for assessing liver function in a subject having or suspected of having or contracting a liver disease may comprise estimating an area under the curve of blood or serum concentrations of the intravenously administered distinguishable cholate compound (AUCiv) comprising a linear regression model. The AUCiv may be estimated by a linear regression model according to equation 11A:

AUC_{IV}=β₀+β_{BW}·BW+β_{PO,20}·C_{PO,20}+β_{PO,60}·C_{PO,60}+β_{HFR,P}·HFR_{P}, Eqn. 11A

wherein β₀ is an intercept coefficient, optionally wherein the intercept coefficient is 161.972; β_{BW} is a body weight coefficient, optionally wherein the body weight coefficient is 0.6459; BW is the subject's body weight in β_{PO,20} is an orally administered distinguishable cholate concentration coefficient at a first time point, optionally wherein the β_{PO,20} is 16.9249; C_{PO,20} is the orally administered distinguishable cholate concentration at the first time point; β_{PO,60} is an orally administered distinguishable cholate concentration coefficient at a second time point, optionally wherein the β_{PO,60} is 89.2405; C_{PO,60} is an orally administered distinguishable cholate concentration at the second time point; β_{HFR,P} is a portal HFR coefficient, optionally wherein the portal HFR coefficient is −0.4755; and HFR_{P} is the subject's measured portal HFR.

In some embodiments, the method for assessing liver function in a subject having or suspected of having or contracting a liver disease may comprise estimating an area under the curve of blood or serum concentrations of an intravenously administered distinguishable cholate compound (AUCiv) comprising obtaining blood or serum sample concentration data of an intravenously administered third distinguishable cholate compound in one of the two time points after the oral administration, and exponential fitting the intravenous concentration data to a systemic cholate clearance curve comprising fast, moderate, and slow phases of clearance over at least about 180 min after the iv administration of the intravenous dose. In some cases, the third distinguishable cholate had been intravenously administered at least about 5 min to about 75 min, 10 min to 70 min, 20 min to 60 min, 25 min to 55 min, 30 min to 50 min, 35 to 45 min, or about 40 min after the first oral dose. In some cases, the blood or serum sample collected at the one time point had been collected within about 90 minutes or less, 75 minutes or less, 60 minutes or less, 45 minutes or less, 30 minutes or less, or about 20 minutes after the intravenous administration.

The exponential fitting to systemic cholate clearance curve fast phase (Y₀) may be calculated according to equation 20:

Y₀=C₀·e^{-k}^{_{fast}}^{·t} Eqn. 20,

wherein t=time (0 to 20 min); C₀ is the initial concentration of intravenously administered distinguishable cholate compound; C₂₀ is the measured 20-minute concentration of intravenously administered distinguishable cholate compound; and k_{fast} is the rate of elimination in the fast phase estimated by equation 18:

$${k}_{fast} = \frac{ln ( {C}_{0} ) - ln ( {C}_{20} )}{20} , \qquad Eqn . 18$$

optionally wherein C₀ is estimated according to equation 17;

$${C}_{0} = \frac{{D}_{IV}}{{V}_{d} \cdot B W} , \qquad Eqn . 17$$

wherein D_{IV} is the intravenous dose of third distinguishable cholate; BW is subject body weight (kg). V_{d} is the volume of distribution (V_{d}) in L per kg body weight, calculated according to equation 16A:

$$T P V = {V}_{d} = \frac{0.07}{\sqrt{B M I / 22}} \cdot ( 1 - Hct ) , \qquad Eqn . 16 A$$

TPV is total plasma volume, BMI is body mass index, and Hct is hematocrit in the subject.

The exponential fitting to systemic cholate clearance curve moderate phase (Yᵢ) may be calculated according to equation 21:

$${Y}_{1} = \frac{{C}_{20}}{{e}^{- {k}_{mod} \cdot 20}} \cdot {e}^{- {k}_{mod} \cdot t} , \qquad Eqn . 21$$

wherein t=time (20-45 min); k_{mod} is the rate of elimination in the moderate phase estimated by equation 19:

k_{mod}=β_{0,mod}+β_{kfast}·k_{fast}+β_{IV,20}·C_{IV,20}+β_{PO,20,mod}·C_{PO,20}+β_{PO,60,mod}·C_{PO,60}

Eqn. 19, wherein β_{0,mod} is the intercept coefficient, optionally wherein the intercept coefficient is 0.0268; β_{kfast} is the k_{fast} coefficient, optionally wherein the k_{fast} coefficient is 0.546; β_{IV,20} is the coefficient of the intravenous distinguishable cholate concentration at the 20 min time point; optionally wherein β_{IV,20} is 0.0045; β_{PO,20,mod} is the coefficient of the oral distinguishable cholate concentration at the 20 min time point, optionally wherein β_{PO,20,mod} is −0.0007; β_{PO,60,mod} is the coefficient of the oral distinguishable cholate concentration at the 60 min time point, optionally wherein β_{PO,60,mod} is −0.0052.

The exponential fitting to systemic cholate clearance curve slow phase (Y₂) may be calculated according to equation 22:

$${Y}_{2} = \frac{{C}_{45}}{{e}^{- {k}_{slow} \cdot 45}} \cdot {e}^{- {k}_{slow} \cdot t} , \qquad Eqn . 22$$

wherein t=time (45-180 min); C₄₅ is the estimated 45-minute concentration of intravenously administered distinguishable cholate; and k_{slow} is the rate of elimination in the slow phase estimated by a mean value from a multiplicity of CLD patients, optionally wherein k_{slow} is 0.018 min⁻¹.

The areas under each of the three exponential curve fits for fast, moderate and slow phases may be calculated by trapezoidal numerical integration and summed to estimate the AUC_{IV}.

A simplified compartmental model is provided for calculating the AUCoral in the subject from orally administered distinguishable cholate compound concentration data at two time points is provided. The simplified compartmental model of oral clearance may comprise estimating compartment volumes of a plurality of compartments in the subject, and flow parameters between the plurality of compartments in the subject. The simplified compartmental model of oral cholate clearance may further include estimating cholate binding and dose administration in the subject.

The plurality of compartments in the subject may comprise at least systemic, portal, and liver compartments.

The estimating compartment volumes of the plurality of compartments may comprise estimating the systemic compartment volume (V_{S}), the portal compartment volume (V_{P}), and the liver compartment volume (V_{L}) in the subject, optionally each compartment volume in liters (L).

The volume of systemic compartment (V_{S}) may be estimated according to equation 4A:

V_{S}=TBV·(1−Hct) Eqn. 4A,
wherein
TBV is total blood volume in the subject according to equation 4:

$$T B V = \frac{0.07 \cdot B W}{\sqrt{B M I / 22}} , \qquad Eqn . 4$$

wherein
   BMI is body mass index (kg/m²) in the subject, and
   BW is body weight (kg) in the subject; and
Hct is the hematocrit in the subject.

The portal compartment volume (VP) may be estimated according to Equation 4B:

V_{P}=0.25·V_{S} Eqn. 4B.

The liver compartment volume (V) may be estimated according to Equation 5A:

V_{L}=(0.275·22.46·BW·d_{L}·fₚₗₐₛₘₐ)/1000 Eqn. 5A,
wherein d_{L} is assumed Liver tissue density of 1.06 g·mL⁻¹.

The simplified compartmental model may comprise estimating of the flow parameters between the plurality of compartments comprising a system of first-order ordinary differential equations 1A to 3:

$$\frac{{dC}_{S}}{dt} = \frac{1}{{V}_{S}} ( - [ {q}_{SP} + {q}_{SL} ] {C}_{S} + {q}_{LS} \cdot {C}_{L} ) ; \qquad Eqn . 1 A$$

$$\frac{dCP}{dt} = \frac{1}{{V}_{P}} ( {D}_{PO} + {q}_{SP} \cdot {C}_{S} - {q}_{PL} \cdot {C}_{P} ) ; \qquad Eqn . 2 A$$

$$and \frac{{dC}_{L}}{dt} = \frac{1}{{V}_{L}} ( - [ {Cl}_{H} \cdot {V}_{L} + {q}_{LS} ] {C}_{L} + {q}_{PL} \cdot {C}_{P} + {q}_{SL} \cdot {C}_{S} ) , \qquad Eqn . 3$$

wherein V is the volume of each of the each of the systemic (V_{S}), portal (V_{P}), and liver (V_{L}) compartments, C is the concentration of cholate in each of the systemic (C_{S}), portal (C_{P}), and liver (C_{L}) compartments, q is the flow rate between compartments, and Cl_{H} is the hepatic clearance.

The simplified compartmental model may comprise estimating total hepatic inflow to the liver (Q_{L}), splanchnic arterial circulation (q_{SP}), hepatic portal venous inflow to the liver (q_{PL}), total hepatic venous return flow to systemic circulation (q_{LS}), and hepatic arterial inflow to the liver (q_{SL}) in the subject.

The estimating total hepatic inflow to the liver (Q_{L}) may comprise both hepatic arterial (q_{SL}) and portal venous (q_{PL}) inflows to the liver according to equation 6A:

Q_{L}=q_{SL}+q_{PL, (L·min}⁻¹) Eqn. 6A,
wherein q_{SL} is rate of hepatic arterial inflow to the liver, q_{SL}=0.25·Q_{L} (L·min⁻¹); and q_{PL} is rate of portal venous inflow to the liver, q_{PL}=q_{SP} (L·min⁻¹), wherein q_{SP} is splanchnic arterial blood flow rate to abdominal intestinal organs, q_{SP}=0.75·Q_{L, init} (L·min⁻¹), optionally wherein the initial estimate for Q_{L} is about 1 L·min⁻¹·kg⁻¹ liver wet weight.

The hepatic clearance (Cl_{H}) may be estimated by equation 7A:

Cl_{H}=Q_{L}·ER Eqn. 7A,
wherein ER is the extraction ratio in the subject and is assumed to be 0.7 for cholate.

In some embodiments, a method for assessing liver function in a subject having or suspected of having or contracting a liver disease is provided, comprising obtaining input data derived from the subject comprising blood or serum sample concentration data of an orally administered distinguishable cholate compound collected from a subject at two time points within 180 minutes after oral administration, body mass index (BMI), optionally hematocrit (Hct), and estimated volume of distribution (Vd) in the subject; fitting the input data to a trained function fitting neural network comprising a training algorithm to generate a multiplicity of output points for oral and intravenous distinguishable cholate clearance curves; generating oral and IV distinguishable cholate clearance curves from the fitted data; measuring AUC_{Oral} and AUC_{IV} values for the subject comprising trapezoidal numerical integration; and calculating one or more indices of hepatic disease in the subject using the AUCoral and/or and AUC_{IV} values wherein the one or more indices is associated with liver function in the subject. In some cases, the Hct in the subject is measured by any appropriate method. In some cases, the Het may be set at an appropriate value in a range between 0-100%, or in a range between 35-50%. In some cases, Hct may be set at 45%.

In some embodiments, a method for assessing liver function in a subject having or suspected of having or contracting a liver disease is provided, comprising obtaining input data derived from the subject comprising blood or serum sample concentration data of an orally administered first distinguishable cholate compound collected from a subject at two time points within 180 minutes after oral administration, blood or serum sample concentration data of an intravenously administered second distinguishable cholate compound collected from a subject at one time point within 180 minutes after intravenous administration, estimated volume of distribution (Vd) in the subject, and estimated initial intravenous distinguishable cholate concentration at 0 minutes of the intravenously administered second distinguishable cholate based on the Vd; fitting the input data to a trained function fitting neural network comprising a training algorithm to generate a multiplicity of output points for oral and intravenous distinguishable cholate clearance curves; generating oral and IV distinguishable cholate clearance curves from the fitted data; measuring AUC_{Oral} and AUC_{IV} values for the subject comprising trapezoidal numerical integration; and calculating one or more indices of hepatic disease in the subject using the AUCoral and/or and AUC_{IV} values wherein the one or more indices is associated with liver function in the subject. In some embodiments, the estimated Vd (L per kg body weight) in the subject may be estimated by equation 16A:

$$T P V = {V}_{d} = \frac{0.07}{\sqrt{B M I / 22}} \cdot ( 1 - Hct ) , \qquad Eqn . 16 A$$

wherein TPV is total plasma volume, BMI is body mass index, and Hct is hematocrit in the subject.

The neural network may be configured for regression tasks and comprises a 2-layer feedforward network comprising hidden layers and an output layer, optionally comprising a sigmoid transfer function in the hidden layers and a linear transfer function in the output layer.

The neural network may comprise a training algorithm had been trained on a training data set comprising a multiplicity of oral and intravenous distinguishable cholate clearance curves estimated by a non-compartmental minimal model (MM) from a combination of normal control subjects and chronic liver disease patients. The training algorithm may be selected from the group consisting of a Levenberg-Marquardt backpropagation, Bayesian Regularization, BFGS Quasi-Newton, Resilient Backpropagation, Scaled Conjugate Gradient, Conjugate Gradient with Powell/Beale Restarts, Fletcher-Power Conjugate Gradient, Polak-Ribiére Conjugate Gradient, One Step Secant, Variable Learning Rate Gradient Descent, Gradient Descent with Momentum, and Gradient Descent training algorithm, and the like.

In some embodiments, the output points for oral and intravenous distinguishable cholate clearance curves comprise 5-minute increments from 0 to 180 minutes after the oral administration, resulting in 37 timepoints for each of the oral and IV clearance curves.

In some cases, the initial intravenous distinguishable cholate compound concentration at 0 minutes is estimated comprising estimating Vd (L per kg body weight) in the subject by equation 16A:

$$T P V = {V}_{d} = \frac{0.07}{\sqrt{B M I / 22}} \cdot ( 1 - Hct ) , \qquad Eqn . 16 A$$

dividing the IV dose by V_{d} times body weight (BW).

A method is provided for assessing liver function in a subject having or suspected of having or contracting a liver disease, comprising:
(a) receiving a plurality of blood or serum samples collected from the subject following oral administration of a dose of a first distinguishable cholate compound (doseₒᵣₐₗ) to the subject and simultaneous intravenous co-administration of a dose of a second distinguishable cholate compound (doseᵢᵥ) to the subject, wherein the samples had been collected over no more than 180 minutes after administration;
(b) quantifying the concentration of the first and the second distinguishable cholate compounds; and
(c) generating individual subject oral and intravenous clearance curves from the concentration of the first and second distinguishable cholate compounds comprising using a computer algorithm curve fitting to model oral and intravenous clearance curves; and computing the area under the individualized oral and intravenous clearance curves (AUCoral) and (AUCiv), respectively, in the subject, wherein the multiplicity of samples comprise blood or serum samples collected from the subject at least 5 time points, and
wherein the generating individual intravenous clearance curve comprises estimating an initial intravenous distinguishable cholate compound concentration in the subject at 0 minutes comprising
estimating Vd (L per kg body weight) in the subject by equation 16A:

$$T P V = {V}_{d} = \frac{0.07}{\sqrt{B M I / 22}} \cdot ( 1 - Hct ) ; \qquad Eqn . 16 A$$

dividing the IV dose by Vd times body weight (BW) to obtain the initial estimated initial intravenous distinguishable cholate concentration; and
constructing the intravenous distinguishable cholate clearance curve comprising the estimated initial intravenous concentration.

The one or more indices of hepatic disease may be selected from the group consisting of portal hepatic filtration rate (HFRp), systemic hepatic filtration rate (HFRs), cholate SHUNT, liver disease severity index (DSI), indexed hepatic reserve (HR_{indexed}), and algebraic hepatic reserve (HR_{algebraic}), in the subject.

The portal hepatic filtration rate (HFRp) in the subject may be calculated by equation 10A:

$$H F {R}_{P} = \frac{{D}_{PO}}{{AUC}_{Oral} \cdot B W} , \qquad Eqn . 10 A$$

wherein D_{PO} is the oral dose of the orally administered distinguishable cholate, and BW is the subject body weight.

The systemic hepatic filtration rate (HFRs) may be calculated according to equation 12:

$$H F {R}_{S} = \frac{{D}_{IV}}{{AUC}_{IV} \cdot B W} , \qquad Eqn . 12$$

wherein D_{IV} is the administered intravenous oral dose of distinguishable cholate, and BW is the subject body weight.

The cholate SHUNT (F) may be calculated according to equation 13:

$$F = \frac{{AUC}_{Oral} \cdot {D}_{IV}}{{AUC}_{IV} \cdot {D}_{PO}} , \qquad Eqn . 13$$

wherein D_{IV} is the intravenous dose of distinguishable cholate, D_{PO} is the oral dose of distinguishable cholate compound, and AUC_{Oral}, and AUC_{IV} are determined by methods as provided herein.

The liver Disease Severity Index (DSI) may be calculated according to equation 14:

$$D S I = A \cdot \sqrt{{( ln [ \frac{{HFR}_{P , max}}{{HFR}_{P}} ] )}^{2} + {( ln [ \frac{{HFR}_{S , max}}{{HFR}_{S}} ] )}^{2}} , \qquad Eqn . 14$$

wherein HFR_{P,max} is the upper limit of portal clearance from a multiplicity of healthy controls; HFR_{S,max} is the upper limit of clearance from a multiplicity of healthy controls; and A is a factor to scale DSI from 0 to 50.

The Hepatic Reserve (HR) may be calculated according to equation 15:

$$H R = 100 - A \cdot \sqrt{{( ln [ \frac{{HFR}_{P , lean}}{{HFR}_{P}} ] )}^{2} + {( ln [ \frac{{HFR}_{S , lean}}{{HFR}_{S}} ] )}^{2}} , \qquad Eqn . 15$$

wherein HFR_{P} and HFR_{S} are indexed to lean controls minus one standard deviation (HFR_{P,lean} and HFR_{S,lean}); and A is a constant to scale HR value from 100 to 0.

The method of the disclosure may include comparing the one or more indices of hepatic disease in the subject to one or more cutoff values as an indicator of the relative hepatic function in the subject. The one or more cutoff values may be derived from one or more normal healthy controls, a group of known patients, or within the subject over time.

The group of known patients may be suffering from any disease or condition. The disease or condition may be selected from the group consisting of a chronic liver disease having a fibrosis stage; portal hypertension; Childs-Turcotte-Pugh (CTP) score A; CTP score B, CTP score C; Model for End-stage Liver Disease (MELD) progression score, primary sclerosing cholangitis (PSC) not listed for transplant; PSC listed for liver transplant; PSC listed for liver transplant without varices; PSC listed for liver transplant with varices; ascites; stomal bleeding; splemomegaly; varices; large varices, variceal hemorrhage; hepatic encephalopathy, decompensation; or liver disease-related death.

The fibrosis stage may be determined by a method comprising liver biopsy or elastography. The liver biopsy may be used to determine Ishak fibrosis score (liver biopsy) of F2 (mild portal fibrosis), F3, F4 (moderate bridging fibrosis), F5 (nodular formation and incomplete cirrhosis), or F6 (cirrhosis).

The one or more indices of hepatic disease may be employed for a purpose selected from the group consisting of determining a need for treatment, predicting response to treatment, monitoring the effectiveness of a treatment, and predicting risk of clinical outcome in the subject.

In some embodiments, the one or more indices of hepatic disease may be employed for a purpose selected from the group consisting of determining a need for treatment, predicting response to treatment, monitoring the effectiveness of a treatment, predicting large esophageal varices, personalized dosing of one or more drugs, and predicting risk of clinical outcome in the subject. The one or more indices of hepatic disease may be used for one or more precision medicine applications, for example, personalization of dosing of one or more of a multitude of drugs, either related to or unrelated to the treatment of hepatic diseases, based the estimated oral bioavailability of the drug.

The method of the disclosure may include providing the one or more indices of hepatic disease to a medical professional for the purpose of developing a treatment plan in the subject. The subject may be a human subject.

The liver disease may be any known chronic liver disease (CLD). The liver disease may be a chronic liver disease selected from the group consisting of chronic hepatitis C (CHC), chronic hepatitis B, metabolic dysfunction-associated alcoholic liver disease (Met-ALD), Alcohol-associated Liver Disease (ALD), steatotic liver disease (SLD), also known as fatty liver disease, Alcoholic SteatoHepatitis (ASH), Alcoholic Hepatitis (AH), metabolic dysfunction-associated steatotic liver disease (MASLD), formerly known as Non-Alcoholic Fatty Liver Disease (NAFLD), steatosis, metabolic dysfunction-associated steatohepatitis (MASH), formerly known as Non-Alcoholic SteatoHepatitis (NASH), autoimmune liver disease, cryptogenic cirrhosis, hemochromatosis, Wilson's disease, alpha-1-antitrypsin deficiency, liver cancer, liver failure, cirrhosis, primary sclerosing cholangitis (PSC), and other cholestatic liver diseases.

The monitoring the need for treatment in the subject may comprise determining the one or more indices of hepatic disease in the subject; and comparing the one or more indices of hepatic disease to one or more cutoff value(s), wherein a change in the one or more indices of hepatic disease compared to cutoff value(s) is indicative of the need for treatment in the subject.

The treatment of chronic liver disease may be selected from the group consisting of antiviral treatments, antifibrotic treatments, antibiotics, immunosuppressive treatments, anti-cancer treatments, ursodeoxycholic acid, farnesoid X receptor ligands, insulin sensitizing agents, interventional treatment, liver transplant, lifestyle changes, dietary restrictions, low glycemic index diet, antioxidants, vitamin supplements, transjugular intrahepatic portosystemic shunt (TIPS), catheter-directed thrombolysis, balloon dilation and stent placement, balloon-dilation and drainage, weight loss, exercise, and avoidance of alcohol. The first distinguishable cholate compound may be a first stable isotope labeled cholate compound. The second distinguishable cholate compound may be a second stable isotope labeled cholate compound. The third distinguishable cholate compound may be a third stable isotope labeled cholate compound.

### BRIEF DESCRIPTION OF THE DRAWINGS

FIG. 1A shows a schematic illustrating DuO cholate liver function test v1.0 parameters for estimating portal clearance using 1 oral cholate dose and 2 blood draws (DuO v1.0) to obtain two orally administered distinguishable cholate (e.g., d4-CA) concentration timepoints. Both versions of the DuO test involve the same analysis method.

FIG. 1B shows a schematic illustrating DuO cholate liver function test v2.0 parameters for estimating portal clearance using 2 distinguishable oral cholate doses and 1 blood draw (DuO v2.0) to obtain two orally administered distinguishable cholate (e.g., d4-CA, 13C-CA) concentration timepoints. Both versions of the DuO test involve the same analysis method.

FIG. 2A shows a schematic illustrating TRIO cholate liver function test v1.0 that estimates both portal and systemic cholate clearances using 1 oral dose+1 IV dose+2 blood draws. (TRIO v1.0). Both versions of the TRIO test use the same analysis method.

FIG. 2B shows a schematic illustrating TRIO cholate liver function test v2.0 that estimates both portal and systemic cholate clearances using 2 oral doses+1 IV dose+1 blood draw (TRIO v2.0). Both versions of the TRIO test use the same analysis method.

FIG. 3 shows an example graph of TRIO cholate liver function test exponential fits to systemic clearance curve of an intravenously administered distinguishable cholate (13C-CA) compared to Minimal Model (MM). A noncompartmental analysis method involving the exponential fits of systemic cholate clearance using only a single IV (e.g., 20 min. 13C-CA concentration) timepoint. In TRIO 1.0, a second sample at 60 min is also analyzed. The exponential fits split the curve into three sections or phases: fast, moderate, and slow (0 to 20 min., 20-45 min., and 45-180 min. for Y₀, Y₁, and Y₂, respectively).

FIG. 4 shows AI DuO and AI TRIO neural network architecture. The neural network outputs selected points from the oral and IV curves estimated by the MM analysis method (i.e., SHUNT V1.0, 5-minute increments from 0 to 180 minutes, resulting in 37 timepoints for both oral and IV curves, rearranged into a 74×1 array). The network was configured for regression tasks and comprises a 2-layer feedforward network with a sigmoid transfer function in the hidden layer and a linear transfer function in the output layer. The hidden layer size was optimized for performance (mean squared error) on a test set.

FIG. 5A shows six graphs illustrating correlation of portal HFR (HFRp) among 2^{nd} generation cholate tests (DuO, TRIO, AI DuO, AI TRIO) and 1^{st} generation SHUNT test results analyzed by the MM (upper panels) and MM_{Vd} methods (lower panels). TRIO graphs are the same for those of DuO and are not shown.

FIG. 5B shows Table 1 with HFRp reproducibility by analysis method as measured by coefficient of variation (CV) and intraclass correlation coefficient (ICC) for patient groups (Controls, N=16; NASH, N=16; HCV, N=16).

FIG. 6A shows eight graphs illustrating correlation of systemic HFR (HFRs) among 2^{nd} generation cholate tests (DuO, TRIO, AI DuO, AI TRIO) and 1^{st} generation SHUNT test results analyzed by the MM and MM_{Vd} methods.

FIG. 6B shows Table 6 with HFRs reproducibility by analysis method as measured by coefficient of variation (CV) and intraclass correlation coefficient (ICC) for patient groups including NASH and HCV (Controls, N=16; NASH, N=16; HCV, N=16).

FIG. 7A shows eight graphs illustrating correlation of SHUNT % among 2^{nd} generation cholate tests (DuO, TRIO, AI DuO, AI TRIO) and 1^{st} generation HepQuant SHUNT test results analyzed by the MM and MM_{Vd} methods.

FIG. 7B shoes Table 8 with SHUNT reproducibility by analysis method as measured by coefficient of variation (CV) and intraclass correlation coefficient (ICC) for patient groups (Controls, N=16; NASH, N=16; HCV, N=16).

FIG. 8A shows eight graphs illustrating correlation of DSI among 2^{nd} generation cholate tests (DuO, TRIO, AI DuO, AI TRIO) and 1^{st} generation SHUNT test results analyzed by the MM and MM_{Vd} methods.

FIG. 8B shows Table 10 with DSI reproducibility by analysis method as measured by coefficient of variation (CV) and intraclass correlation coefficient (ICC) for patient groups (Controls, N=16; NASH, N=16; HCV, N=16).

FIG. 9A shows eight graphs illustrating correlation of indexed hepatic reserve (HR_{indexed}) among 2^{nd} generation cholate tests (DuO, TRIO, AI DuO, AI TRIO) and 1^{st} generation SHUNT cholate test results analyzed by the MM and MM_{Vd} methods.

FIG. 9B shows Table 12 with indexed hepatic reserve (HR_{indexed}) reproducibility by analysis method as measured by coefficient of variation (CV) and intraclass correlation coefficient (ICC) for patient groups (Controls, N=16; NASH, N=16; HCV, N=16).

FIG. 10A shows Table 13A with reproducibility and diagnostic performance for prediction of large esophageal varices. For the measurement of DSI, MM_{Vd} and TRIO (v 1.0) cholate test versions demonstrated similar ICCs but improved CVs relative to the MM method. For measurement of SHUNT %, diagnostic performance based on AUROCs for MM_{Vd} and TRIO (v 1.0) was equivalent to MM in most cases and improved in MM_{Vd}.

FIG. 10B shows DSI values measured by DuO cholate test in Child-Pugh A cirrhosis subjects and control subjects. DSI from lean and overweight controls was plotted alongside subjects with no, small, or large esophageal varices (LEVs). DSI cutoff of 18.3 is shown as a horizontal line. A monotonic, stepwise increase in DSI with increasing risk for LEVs is exhibited.

FIG. 10C shows a graph of DSI values measured by DuO cholate test in Child-Pugh A cirrhosis subjects and control subjects vs. probability of LEVs based on DSI. DSI cutoff of 18.3 is shown as a vertical line. DSI from DuO demonstrated significant association with finding LEVs at endoscopy (p<0.001).

FIGS. 11A-D show pharmacokinetic clearance curves for various versions of the HepQuant liver function tests on a NASH subject with F3 fibrosis.

FIG. 11A shows pharmacokinetic clearance curves for original cholate SHUNT liver function test (SHUNT V1.0) which measures the concentration of orally administered (PO) d4-CA and intravenously (IV) administered 13C-CA in systemic circulation across 90 minutes.

FIG. 11B shows pharmacokinetic clearance curves for cholate SHUNT V1.1 liver function test using the patient's physical characteristics to calculate the initial 13C-CA concentration, eliminating the need for sampling at 5 minutes.

FIG. 11C shows pharmacokinetic clearance curves for cholate SHUNT V2.0 liver function test which calculates oral and IV cholate clearances using samples collected at 20 and 60 minutes.

FIG. 11D shows pharmacokinetic clearance curves for cholate DuO liver function test which calculates portal clearance from samples collected at 20 and 60 minutes and systemic clearance by derived IV.

FIG. 12 shows a schematic of a compartmental model of dual cholate clearance describing the transfer of cholate between systemic, portal, and liver compartments. D_{IV}=intravenous dose of 13C cholate (SHUNT V2.0); D_{PO}=oral dose of d4-CA (SHUNT V2.0 and DuO); q_{SP}=splanchnic arterial flow rate; q_{PS}=portal systemic shunt flow rate; q_{PL}=portal venous flow rate; q_{LS}=hepatic return to systemic circulation flow rate; q_{SL}=hepatic arterial flow rate; Cl_{H}=hepatic clearance. Reprinted from Translational Research, Volume 252, McRae, M. P. et al., Compartmental model describing the physiological basis for the HepQuant SHUNT test, Pages 53-63, Copyright (2023), with permission from Elsevier.

FIG. 13 shows two graphs of fitting functions for estimation of DSI (left panel) and portal HFR (right panel) using STAT. Scatter dots show values for the PSC Study and the REPRO Study (control, NASH, and HCV groups).

FIG. 14A-D shows Equivalence of next-generation simplified cholate liver function tests to SHUNT V1.1 in terms of DSI by each study individually and across all studies. FIG. 14 A shows a plot of Two one-sided t-test (TOST) analysis of SHUNT V2.0 for DSI. FIG. 14 B shows Bioequivalence analysis of SHUNT V2.0 for DSI. FIG. 14C shows TOST analysis of DuO for DSI. FIG. 14 D shows Bioequivalence analysis of DuO for DSI. Error bars represent 90% confidence intervals. Dotted vertical lines indicate equivalence bounds (TOST) or bioequivalence limits. REPRO=HepQuant Reproducibility Study; PSC=Primary Sclerosing Cholangitis Study; HALT-C=Hepatitis C Antiviral Long-Term Treatment Against Cirrhosis Trial; ALL=all studies combined.

FIG. 15A-15B show Equivalence analysis of SHUNT V2.0 versus SHUNT V1.1 for DSI. Bland-Altman plot (FIG. 15A) shows a graph of the mean of methods versus the difference between methods with bias (solid black line), 95% confidence intervals of the mean difference (dotted lines), and equivalence bands (red lines). The correlation plot (FIG. 15B) shows the DSI values for both methods and Deming regression (solid line). Each point represents values derived from a single test across all studies in the analysis.

FIG. 16A-168 show Equivalence analysis of DuO versus SHUNT V1.1 for DSI. Bland-Altman plot (FIG. 16A) shows the mean of methods versus the difference between methods with bias (solid black line), 95% confidence intervals of the mean difference (dotted lines), and equivalence bands (red lines). The correlation plot (FIG. 16B) shows the DSI values for both methods and Deming regression (solid line). Each point represents values derived from a single test across all studies in the analysis.

FIG. 17A-17B show Equivalence analysis of STAT versus SHUNT V1.1 for DSI. Bland-Altman plot (FIG. 17A) shows the mean of methods versus the difference between methods with bias (solid black line), 95% confidence intervals of the mean difference (dotted lines), and equivalence bands (red lines). The correlation plot (FIG. 17B) shows the DSI values for both methods and Deming regression (solid line). Each point represents values derived from a single test across all studies in the analysis.

FIG. 18A-18B show Equivalence analysis of DuO versus SHUNT V1.1 for Hepatic Reserve. Bland-Altman plot (FIG. 18A) shows the mean of methods versus the difference between methods with bias (solid black line), 95% confidence intervals of the mean difference (dotted lines), and equivalence bands (red lines). The correlation plot (FIG. 18B) shows the Hepatic Reserve values for both methods and Deming regression (solid line). Each point represents values derived from a single test across all studies in the analysis.

FIG. 19A-D show Equivalence of next-generation simplified liver function tests to SHUNT V1.1 in terms of Hepatic Reserve (HR) by each study individually and across all studies. FIG. 19A shows Two one-sided t-test (TOST) analysis of SHUNT 2.0 for HR. FIG. 19B shows Bioequivalence analysis of SHUNT 2.0 for HR. FIG. 19C shows TOST analysis of DuO for HR. FIG. 19D shows Bioequivalence analysis of DuO for HR. Error bars represent 90% confidence intervals. Dotted red line indicates equivalence bounds (TOST) or bioequivalence limits. REPRO=HepQuant Reproducibility Study; PSC=Primary Sclerosing Cholangitis Study; HALT-C=Hepatitis C Antiviral Long-Term Treatment Against Cirrhosis Trial; ALL=all studies combined.

FIG. 20A-20B show Equivalence analysis of SHUNT V1.1 versus SHUNT V1.0 for DSI. Bland-Altman plot (FIG. 20A) shows the mean of methods versus the difference between methods with bias (solid black line), 95% confidence intervals of the mean difference (dotted lines), and equivalence bands (red lines). The correlation plot (FIG. 20B) shows the DSI values for both methods and Deming regression (solid line). Each point represents values derived from a single test across all studies in the analysis.

FIG. 21A shows a graph of PSC progressor groups slow, intermediate, and rapid progressors as age in years vs. DSI value from DuO cholate test.

FIG. 21B shows graph of PSC progressor groups slow, intermediate, and rapid progressors as age in years vs. SHUNT % value from DuO cholate test.

FIG. 22A shows a graph of SHUNT % vs. probability of portal hypertension for PSC study subjects.

FIG. 22B shows a graph of SHUNT % vs. probability of varices for PSC study subjects.

FIG. 23 shows a timeline of an open-label active Resmetirom treatment arm in patients with well-compensated (Child-Pugh A [CP-A]) NASH cirrhosis.

FIG. 24A shows pharmacokinetic clearance curves for cholate SHUNT V2.0 liver function test which calculates oral and IV cholate clearances using samples collected at 20 and 60 minutes.

FIG. 24B shows pharmacokinetic clearance curves for cholate DuO liver function test which calculates portal clearance from samples collected at 20 and 60 minutes and systemic clearance by derived IV.

FIG. 25A shows Risk ACE calculated using DuO cholate test after 28 weeks of Resmetirom compared to baseline. Risk ACE from DuO decreased with resmetirom treatment in 21 of 23 subjects.

FIG. 25B shows Risk ACE calculated using DuO cholate test after 48 weeks of Resmetirom compared to baseline. At 48 weeks, Risk ACE decreased in 19 of 23 subjects.

FIG. 26A shows Risk ACE calculated using SHUNT V2.0 cholate test after 28 weeks of Resmetirom compared to baseline. Risk ACE from SHUNT V2.0 decreased in 20 of 23 subjects at 28 weeks.

FIG. 26B shows Risk ACE calculated using SHUNT V2.0 cholate test after 48 weeks of Resmetirom compared to baseline. Risk ACE from SHUNT V2.0 decreased in 19 of 23 subjects at 48 weeks.

### DETAILED DESCRIPTION OF THE INVENTION

Current noninvasive liver tests are surrogates for fibrosis and do not measure function. The HepQuant platform of noninvasive cholate liver function tests uniquely assesses both liver function and physiology through the hepatic uptake of stable isotopes of cholate. However, the prototypical HepQuant SHUNT test (SHUNT V1.0) described in U.S. Pat. Nos. 8,613,904, and 8,778,299 can be cumbersome to administer, requiring intravenous and oral administration of cholate and six timed peripheral venous blood samples over 90 minutes.

To alleviate the burden of SHUNT test administration, the inventors explored whether an oral only (DuO) cholate liver function test and other simplified versions of the test could provide reproducible measurements of liver function while maintaining equivalency of results. The simplified test versions include SHUNT V1.1 (oral and IV dosing but 4 blood samples), SHUNT V2.0 (oral and IV dosing but only 2 blood samples over 60 minutes), and DuO (oral dosing and 2 blood samples over 60 minutes).

The simplified test methods, particularly SHUNT V2.0 and DuO are easier to administer and less invasive, thus, having the potential to be more widely accepted by care providers administering the test and by patients receiving the test. The HepQuant Test versions are displayed in Table 1A showing an overview of analysis methods in terms of number and administration route of cholate isotope doses, number of blood samples, and duration of the testing period in minutes.

TABLE 1A
Overview of Simplified Cholate Liver Function Tests

| | | | Duration of | Alternative |
|---|---|---|---|---|
| | No. of isotope | No. of | testing | Name/Analysis |
| Test Version | doses | samples | period | Method |
| SHUNT V1.0 | 1 oral + 1 IV | 6 | 90 min. | MM, Minimal |
| | | | | Model |
| SHUNT V1.1 | 1 oral + 1 IV | 4 | 90 min. | MM_{Vd}, |
| | | | | Minimal Model |
| | | | | Vd |
| SHUNT V2.0 | 1 oral + 1 IV | 2 | 60 min. | TRIO V1.0, |
| | | | | TRIO |
| SHUNT V3.0 | 2 oral + 1 IV | 1 | 60 min. | TRIO V2.0 |
| DuO | 1 oral | 2 | 60 min. | DUO, |
| | | | | DuO V1.0 |
| DuO V2.0 | 2 oral | 1 | 60 min. | DuO V2.0 |
| STAT | 1 oral | 1 | 60 min. | cholate STAT, |
| | | | | STAT V1.0 |

The first-generation cholate SHUNT liver function test (SHUNT V1.0) measures portal and systemic clearances simultaneously, comprising: an intravenous (IV) dose of carbon-13-labeled cholate (13C-CA), a simultaneous oral dose of deuterium-labeled cholate (d4-CA), and 6 peripheral venous blood draws over 90 minutes. The SHUNT V1.0 test may be analyzed by the MM method.

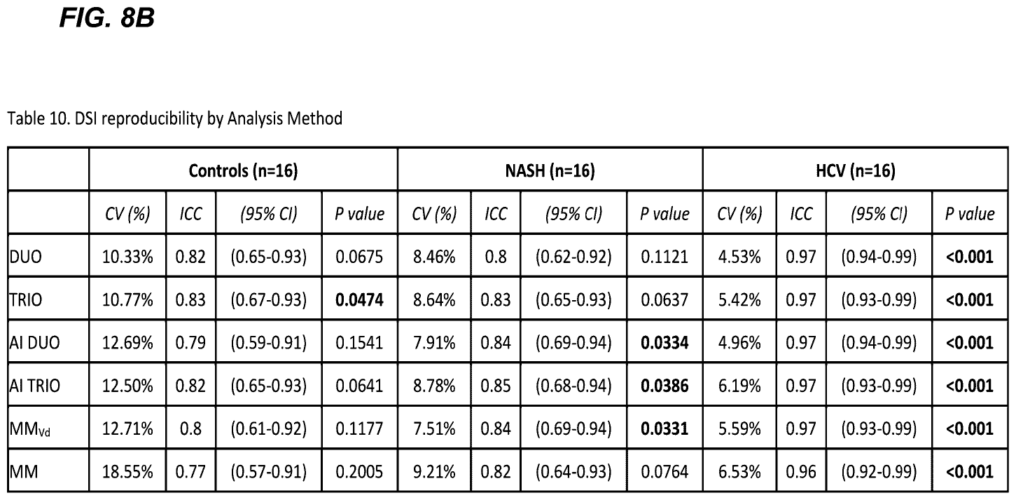

The cholate SHUNT V1.0 (FIG. 11A) liver function test and MM analysis method describes IV clearance by noncompartmental exponential fits and oral clearance by a cubic spline fit to calculate the areas under the IV and oral curves (AUC_{IV}, AUC_{Oral}), respectively. Everson et al. Portal-systemic shunting in patients with fibrosis or cirrhosis due to chronic hepatitis C: the minimal model for measuring cholate clearances and shunt. Aliment Pharmacol Ther. 2007; 26:401-10. Peripheral venous blood is obtained at 0, 5, 20, 45, 60, and 90 minutes within specified time windows, and concentrations of administered cholates are measured by liquid chromatography/mass spectrometry (LC/MS). After IV injection, residual 13C-CA in the sampling catheter and sampling blood beyond the time window, especially for the 5-minute sample (±1 minute), were sources of error in a minority of tests but may have compromised results in those tests.

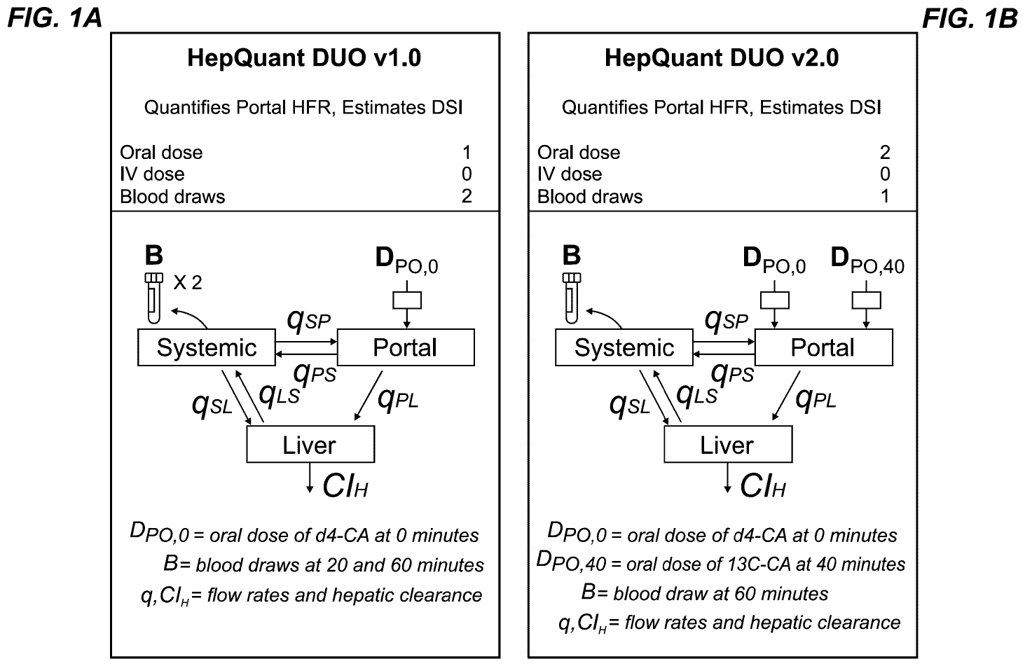

The cholate SHUNT V1.1 (FIG. 1I B) liver function test is a cholate liver function test that employs an MM_{Vd} analysis method to eliminate the 5-minute sample from the calculations. The initial 13C-CA concentration at 0 minutes is calculated from administered dose and estimation of blood volume from body mass index (BMI). Lemmens et al., Estimating blood volume in obese and morbidly obese patients. Obes Surg. 2006; 16:773-6. The 5-minute 13C-CA concentration is then estimated by log-linear regression between the 0- and 20-minute concentrations, and the 5-minute oral concentration is approximated as 15% of the 20-minute d4-CA concentration. The AUC_{IV} and AUC_{Oral} are then estimated by the same Minimal Model equations as in SHUNT V1.0.

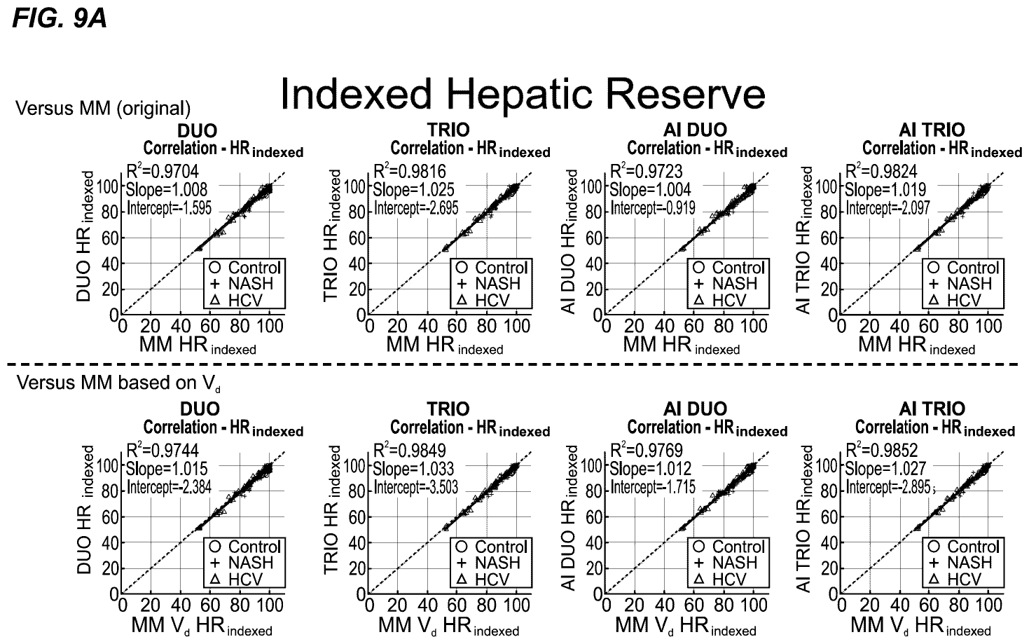

The cholate SHUNT V2.0 (FIG. 11C)(also known as TRIO V1.0) is a cholate liver function test that implements (1) a compartmental model of portal cholate clearance that uses assumptions of liver flow and physiology to predict oral clearance curves and (2) noncompartmental exponential fits to systemic cholate clearance. The compartmental model (FIG. 12) for measuring portal clearance describes the flow between systemic (S), portal (P), and liver (L) compartments represented by a system of first-order ordinary differential equations (Equations 1B, 213, and 3), where q is the flow rate between compartments, V is the volume of the compartment, C is the concentration of d4-CA in the compartment, Cl_{H} is the hepatic clearance, and D_{PO,rate} is the rate of orally administered d4-CA entering the portal compartment.

$$\frac{{dC}_{S}}{dt} = \frac{1}{{V}_{S}} ( - [ {q}_{SP} + {q}_{SL} ] {C}_{S} + {q}_{LS} \cdot {C}_{L} + {q}_{PS} \cdot {C}_{P} ) \qquad Eqn . 1 B$$

$$\frac{{dC}_{P}}{dt} = \frac{1}{{V}_{P}} ( {D}_{PO , rate} + {q}_{SP} \cdot {C}_{S} - {q}_{PL} \cdot {C}_{P} - {q}_{PS} \cdot {C}_{P} ) \qquad Eqn . 2 B$$

$$\frac{{dC}_{L}}{dt} = \frac{1}{{V}_{L}} ( - [ {Cl}_{H} \cdot {V}_{L} + {q}_{LS} ] {C}_{L} + {q}_{PL} \cdot {C}_{P} + {q}_{SL} \cdot {C}_{S} ) \qquad Eqn . 3$$

The system of ordinary differential equations is solved numerically, and the concentration of d4-CA in the systemic compartment is integrated over 180 minutes to calculate the AUC_{Oral}.

The noncompartmental analysis for measuring systemic clearance involves the exponential fits of systemic cholate clearance using the 20- and 60-minute 13C-CA concentrations. The exponential fits split the curve into three clearance phases: fast (Y₀), moderate (Y₁), and slow (Y₂). Equations 4C, 5B, and 6B define the systemic concentration of 13C-CA through time.

$${Y}_{0} = {C}_{0} \cdot {e}^{- {k}_{fast} \cdot t} \qquad Eqn . 4 C$$

$${Y}_{1} = \frac{{C}_{20}}{{e}^{- {k}_{mod} \cdot {T}_{20}}} \cdot {e}^{- {k}_{mod} \cdot t} \qquad Eqn . 5 B$$

$${Y}_{2} = \frac{{C}_{60}}{{e}^{- {k}_{slow} \cdot {T}_{60}}} \cdot {e}^{- {k}_{slow} \cdot t} \qquad Eqn . 6 B$$

Here, t is time (0-20, 20-60, and 60-180 minutes for Y₀, Y₁, and Y₂, respectively), C₀ is the initial concentration of 13C-CA, C₂₀ and C₆₀ are the measured 20- and 60-minute concentrations of 13C-CA, and T₂₀ and T₆₀ are the actual collection times of the 20- and 60-minute blood sample. The systemic concentration of 13C-CA is then integrated to calculate the AUC_{IV}. See the Example 2 for a more detailed description of both the compartmental analysis for portal clearance and the noncompartmental analysis for systemic clearance.

The DuO test (FIG. 11D) (also known as DuO V1.0 or dual sample oral cholate challenge test) is an oral-only cholate liver function test involving administration of one oral dose (d4-CA at 0 minutes) and collection of two blood samples (e.g., at 20 and 60 minutes) to quantify portal HFR and estimate systemic HFR. These analyses involve the same compartmental model of portal cholate clearance as SHUNT V2.0. The systemic clearance is calculated by first estimating the derived IV concentrations at 20 and 60 minutes using linear regression models (Table 14) with body weight, BMI, the actual time of the 20-minute blood sample, and d4-CA concentrations at 20 and 60 minutes (see Example 2 for a detailed description of the linear models and the regression coefficients). The derived IV concentrations are then used in the same noncompartmental analysis as SHUNT V2.0.

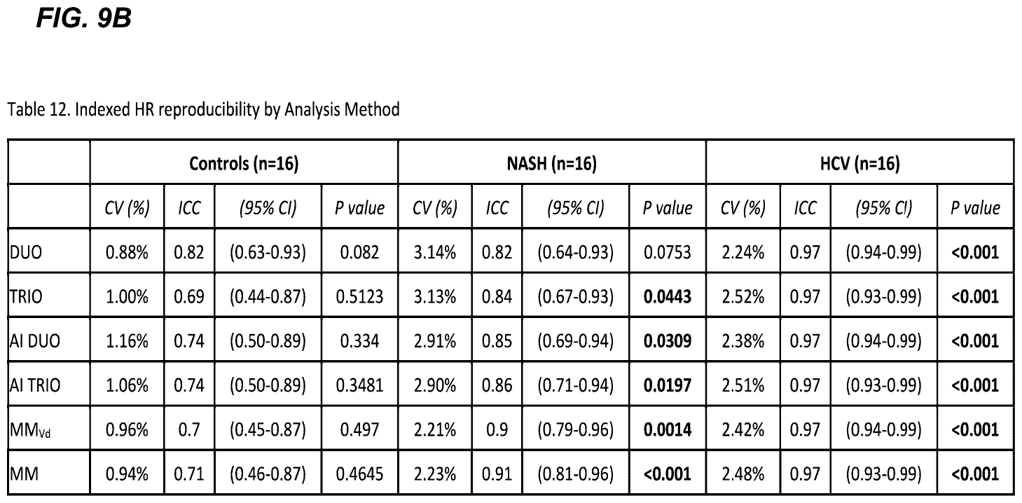

The STAT V1.0 test (also known as the cholate STAT test or STAT) is an oral-only cholate liver function test involving administration of one oral dose (e.g., d4-CA at 0 minutes) and collection of a single blood or serum sample (e.g., at 60 minutes). The STAT test is simply the d4-CA concentration at 60 minutes normalized to 75 kg body weight by the calculation ([d4-CA]×(kg body weight/75 kg)). The value derived from the STAT test may be used by itself or in the estimation of portal HFR and DSI (FIG. 13).

Liver Function Test Parameters

The following parameters are calculated by the SHUNT and DuO tests and have previously demonstrated associations with liver function:

Portal hepatic filtration rate (HFR_{P}) is the portal clearance adjusted for body weight (units mL min⁻¹ kg⁻¹) calculated by Equation 7B, where D_{PO} is the oral dose.

$$H F {R}_{P} = \frac{{D}_{P O}}{{AUC}_{Oral} \cdot B W} \qquad Eqn . 7 B$$

Systemic hepatic filtration rate (HFR_{S}) is the measured systemic clearance adjusted for body weight (units mL min⁻¹ kg⁻¹) calculated by Equation 8B, where D_{IV} is the intravenous dose.

$$H F {R}_{S} = \frac{{D}_{IV}}{A U {C}_{IV} \cdot B W} \qquad Eqn . 8 B$$

SHUNT/is the estimated absolute bioavailability of orally administered d4-CA, or equivalently the ratio of systemic and portal HFRs (Equation 9B).

$$SHUNT \% = \frac{{AUC}_{Oral} \cdot {D}_{IV}}{{AUC}_{IV} \cdot {D}_{PO}} = \frac{{HFR}_{S}}{{HFR}_{P}} \qquad Eqn . 9 B$$

DSI is a score indicative of overall liver function comprising both portal and systemic HFR. The DSI conveys unique information regarding fibrosis stage and clinical stages of cirrhosis, and modeling these outputs for prediction of clinical outcomes produced the DSI in Equation 10B [8, 10-12]. Here, HFR_{P,max} and HFR_{S,max} are the upper limits of clearance for healthy controls, and A is a factor to scale DSI from 0 to 50.

$$DSI = A \cdot \sqrt{{( ln [ \frac{{HFR}_{P , max}}{{HFR}_{P}} ] )}^{2} + {( ln [ \frac{{HFR}_{S , max}}{{HFR}_{S}} ] )}^{2}} \qquad Eqn . 10 B$$

Hepatic Reserve is a numerical index representing overall hepatic health with values between 0 and 100 (Equation 11B). Here, Hepatic Reserve uses HFR_{P} and HFR_{S} indexed to lean controls minus one standard deviation (HFR_{P,lean} and HFR_{S,lean}) with scaling factor, A, to scale Hepatic Reserve from 100 to 0. Any HFR_{P} value above HFR_{P,lean} is set to the HFR_{P,lean} value, and similarly any HFR_{S} value above HFR_{S,lean} is set to the HFR_{S,lean} value. Thus, any patients with both HFR values greater than these limits are calculated to have an HR of 100.

$$HR = 100 - A \cdot \sqrt{{( ln [ \frac{H F {R}_{P , leαn}}{H F {R}_{P}} ] )}^{2} + {( ln [ \frac{{HFR}_{S , lean}}{{HFR}_{S}} ] )}^{2}} \qquad Eqn . 11 B$$

RCA-20 is the residual 13C-CA at 20 minutes as a percent of the initial estimated 13C-CA immediately following the IV administration.

Risk-ACE (clinical events per person-year) is derived from a Poisson regression model as a function of DSI.

STAT may be derived from SHUNT, DuO, or STAT tests as the d4-CA concentration at 60 minutes normalized to 75 kg body weight.

Reproducibility of test parameters DSI, SHUNT %, Portal HFR, Systemic HFR, Hepatic Reserve, RCA-20, Risk ACE, STAT, STAT estimated DSI, and STAT estimated Portal HFR as calculated using SHUNT V1.0, SHUNT V1.1, SHUNT V2.0 and DuO analysis methods provided herein is shown in Example 2.

The present disclosure provides a series of second-generation liver function tests which provide accurate measures of liver function that correlate with first generation liver function tests (cholate STAT, FLOW, and SHUNT tests), but which are significantly simplified in terms of the test administration.

Liver function test parameters (DSI, HFR, SHUNT, HR, etc.) require accurate and reliable measurements of the area under the oral and IV distinguishable cholate clearance curves (AUC_{Oral}, AUC_{IV}).

The present disclosure provides simplified DuO and TRIO liver function tests, requiring only a limited number of datapoints (only 2 oral data points for DuO, 2 oral plus 1 IV data points for TRIO), as well as specialized analysis techniques to fit the systemic and portal clearance curves therefrom. Analysis methodologies are provided herein for each test.

Briefly, the methods provided herein include the cholate DuO liver function test, cholate TRIO liver function test, cholate AI liver function test, and the cholate Vd-based Minimal Model (MM_{Vd}) liver function test.

The cholate DuO liver function test, also known as Dual Sample Oral Cholate Challenge Test (also known as “DuO”, “DUO”, “DuO test” “DuO cholate test”, “HepQuant DUO”, “HepQuant DuO”) is a compartmental model of portal cholate clearance that uses assumptions of liver flow and physiology to predict oral clearance curves comprising measuring only two (e.g., 20 min. and 60 min.) orally administered distinguishable cholate (e.g., d4-CA) concentration timepoints (i.e., DuO liver function test v1.0). The DuO v1.0 test comprises administration of 1 oral dose of a distinguishable cholate (e.g., d4-CA at 0 min.), and collection of 2 blood draws at first and second time points (e.g., 20 and 60 min.). The DuO v2.0 test comprises administration of 2 oral doses of first and second distinguishable cholates, respectively, at first and second time points (e.g., d4-CA at 0 min., 13C-CA at 40 min.), and collection of 1 blood draw at a single time point (e.g., 60 min.). The DuO test quantifies portal HFR and estimates systemic HFR, DSI, SHUNT %, and Hepatic Reserve (HR) using only oral dose(s). The DuO dual oral cholate clearance tests do not require measurement of an intravenously administered distinguishable cholate.

The cholate SHUNT V2.0 liver function test (also known as “TRIO V1.0 test”, “cholate TRIO V1.0 liver function test”, “HepQuant TRIO”) is a noncompartmental method comprising exponential fits of systemic cholate clearance comprising measuring a single (e.g., 20 min.) intravenously administered distinguishable cholate (e.g., 13C-CA) concentration timepoint (i.e., TRIO liver function test v1.0). The SHUNT V2.0 test (TRIO V1.0 test) comprises administration of 1 oral dose of a first distinguishable cholate at a first time point (e.g., d4-CA at 0 min.) and 1 intravenous dose of a second distinguishable cholate at a second time point (e.g., 13C-CA at 0 min.), and collection of 2 blood draws (e.g., at 20 and 60 min.).

The cholate SHUNT V3.0 liver function test (also known as TRIO V2.0 test, cholate TRIO V2.0 liver function test test) comprises administration of 2 oral doses of 2 different distinguishable cholates at first and second time points (e.g., d4-CA at 0 min., d2-CA at 40 min. or d5-CA at 40 min.) and 1 intravenous dose of a third distinguishable cholate at a third time point (e.g., 13C-CA at 40 min.), and collection of 1 blood draw (e.g., 60 min.). The second and third time points may be simultaneous, or substantially simultaneous. The TRIO test quantifies portal HFR, systemic HFR, DSI, SHUNT %, and Hepatic Reserve (HR) using oral and intravenously administered cholate doses.

The cholate AI liver function test comprises function fitting neural networks trained to predict response curves as calculated by the Minimal Model approach when provided a limited set of inputs (i.e., DuO v1.0 and TRIO V1.0 cholate tests).

The SHUNT V1.1 also known as cholate SHUNT Vd-based Minimal Model (MM_{Vd}) liver function test is an adaptation of the previously validated Minimal Model method which uses the volume of distribution (Vd) estimated from body mass index (BMI) to inform the initial 13C-CA concentration at 0 min.

### Definitions

As used herein, “a” or “an” may mean one or more than one of an item.

The term “about” when referring to any numerical parameter means +/−10% of the numerical value. For example, the phrase “about 60 minutes” refers to 60 minutes+/−6 minutes.

All patents, patent applications and publications referred to herein are incorporated by reference herein in their entirety.

The term “accuracy” (measurement) when used herein refers to closeness of agreement between a measured quantity value and a true quantity value of a measurand.

The term “acceptability” as used herein is based on individual criteria that set minimal operational characteristics for a measurement procedure.

The term “precision” as used herein refers to closeness of agreement between independent test/measurement results obtained under stipulated conditions.

The term “trueness” as used herein refers to the closeness of agreement between the expectation of a test result or a measurement result and a true value.

The term “measurand” is used when referring to the quantity intended to be measured instead of analyte (component represented in the name of a measurable quantity).

The term “verification” as used herein focuses on whether specifications of a measurement procedure can be achieved, whereas the term “validation” verifies that the procedure is fit for an intended purpose.

The term “measurement procedure” refers to a detailed description of a measurement according to one or more measurement principles and to a given measurement method, based on a measurement model and including any calculation to obtain a measurement result.

As used herein “clearance” may mean the removing of a substance from one place to another.

As used herein, the term “simultaneously” when referring to 2 or more events refers to occurring within 10 minutes or less, 5 minutes, or within about 3 minutes of each other.

As used herein the terms, “patient”, “subject” or “subjects” include but are not limited to humans, the term may also encompass other mammals, or domestic or exotic animals, for example, dogs, cats, ferrets, rabbits, pigs, horses, cattle, birds, or reptiles.

The acronym “HALT-C” refers to the Hepatitis C Antiviral Long-term Treatment against Cirrhosis trial. The HALT-C trial was a large, prospective, randomized, controlled trial of long-term low dose peg interferon therapy in patients with advanced hepatitis C who had not had a sustained virologic response to a previous course of interferon-based therapy. An NIH-sponsored Hepatitis C Antiviral Long-Term Treatment against Cirrhosis (HALT-C) Trial examined whether long-term use of antiviral therapy (maintenance treatment) would slow the progression of liver disease. In noncirrhotic patients who exhibited significant fibrosis, effective maintenance therapy was expected to slow or stop histological progression to cirrhosis as assessed by serial liver biopsies. However, tracking disease progression with biopsy carries risk of complication, possibly death. In addition, sampling error and variation of pathologic interpretation of liver biopsy limits the accuracy of histologic assessment and endpoints. The histologic endpoint is less reliable because advanced fibrosis already exists and changes in fibrosis related to treatment or disease progression cannot be detected. Thus, standard endpoints for effective response to maintenance therapy in cirrhotic patients are prevention of clinical decompensation (ascites, variceal hemorrhage, and encephalopathy) and stabilization of liver function as measured clinically by Childs-Turcotte-Pugh (CTP) score. However, clinical endpoints and CTP score were known to be insensitive parameters of disease progression. Dual isotope techniques employing distinguishable cholates were used in development of the SHUNT test and used in conjunction with the HALT-C trial. The term “SHUNT test” refers to a previously disclosed QLFT (quantitative liver function test) used as a comprehensive assessment of hepatic blood flow and liver function. The SHUNT test is used to determine clearance of orally and intravenously administered distinguishable cholic acids in subjects with and without chronic liver disease. SHUNT fraction or percent quantifies the spillover of the PO d4-cholate into the systemic circulation from the ratio of the clearance of the intravenously administered 13C-cholate to the clearance of the orally administered d4-cholate. In the SHUNT test, at least 5 blood samples are analyzed which have been drawn from a patient at intervals over a period of at least about 90 minutes after oral and intravenous administration of differentiable cholates. The SHUNT test is disclosed in Everson et al., U.S. Pat. No. 8,613,904, which is incorporated herein by reference. These studies demonstrated reduced clearance of cholate in patients who had either hepatocellular damage or ponosystemic shunting. The “SHUNT test value” refers to a number (in %). The term “SHUNT %” represents a quantitative measurement of portal-systemic shunting. SHUNT % is a measurement of the percentage of spillover of the orally administered d4-cholate. The first-pass hepatic elimination of cholate in percent of orally administered cholate is defined as (100%−SHUNT). SHUNT test methods are disclosed in U.S. Pat. Nos. 8,613,904, 9,639,665, 8,778,299, 9,417,230, and 10,215,746, each of which is incorporated herein by reference in its entirety. Analysis of samples for stable isotopically labeled cholates is performed by, e.g., GC-MS, following sample derivitization, or LC-MS, without sample derivitization, or LC-MS/MS, or MS/MS as disclosed herein. The ratio of the AUCs of orally to intravenously administered cholic acid, corrected for administered doses, defines cholate shunt. The cholate shunt can be calculated using the formula: AUCₒᵣₐₗ/AUCᵢᵥ×Doseᵢᵥ/Doseₒᵣₐₗ×100%, wherein AUCₒᵣₐₗ is the area under the curve of the serum concentrations of the orally adminstered cholic acid and AUCiv is the area under the curve of the intravenously administered cholic acid.

The SHUNT test allows measurement of first-pass hepatic elimination of bile acids from the portal circulation. Flow-dependent, first pass elimination of bile acids by the liver ranges from about 60% for unconjugated dihydroxy, bile acids to about 95% for glycine-conjugated cholate. Free cholate, used herein has a reported first-pass elimination of approximately 80% which agrees closely with previously observed first pass elimination in healthy controls of about 83%. After uptake by the liver, cholic acid is efficiently conjugated to either glycine or taurine and secreted into bile. Physicochemically cholic acid may be easily separated from other bile acids and bile acid or cholic acid conjugates, using chromatographic methods.

The term “Cholate Elimination Rate”, kₑₗᵢₘ min⁻¹ represents the first phase of elimination of the intravenously administered 13C-cholate, calculation from Ln/linear regression of [13C-cholate] versus time (using only the 5- and 20-minute time points). Intravenously administered 13C-cholate is rapidly delivered to the liver via the hepatic artery. In contrast, the same 13C-cholate slowly transits to the liver via the portal vein due to the capacitance of the splanchnic vascular bed. Thus, the first phase of cholate elimination is more dependent upon clearance from the hepatic artery than from portal vein.

The term “Volume of distribution”, V_{d}, (L kg⁻¹) represents the body's volume into which cholate is distributed.

The acronym “IV” or “iv” refers to intravenous route of administration.

The acronym “PO” refers to per oral route of administration.

The acronym “PHM” refers to perfused hepatic mass.

The acronym “SF” refers to shunt fraction, for example, as in liver SF, or cholate SF.

The acronym “ROC” refers to receiver operating characteristic. The ROC curve is a graphical plot which illustrates performance of a binary classifier system as its discrimination threshold is varied. It is created by plotting the fraction of true positives out of the positives (TPR=true positive rate) vs. the fraction of false positives out of the negatives (FPR=false positive rate), at various threshold settings. Sensitivity is the probability of a positive test result, or of a value above a threshold, among those with disease. Sensitivity is defined as the true positive rate (TPR): TPR=TP/P=TP/(TP+FN). False positive rate (FPR) is FPR=FP/N=FP/(FP+FN). Accuracy (ACC) is defined as ACC=(TP+TN)/(P+N). Specificity is the probability of a negative test result, or a value below a threshold, among those without disease. Specificity (SPC), or true negative rate (TN) is defined as SPC=TN/N=TN/(FP+TN)=1−FPR. Positive prediction value (PPV) is defined as: PPV=TP/(IP+FP). Negative predictive value (NPV) is defined as NPV=TN/(TN+FN).

The c-statistic is the area under the ROC curve, or “AUROC” (area under receiver operating characteristic curve) and ranges from 0.5 (no discrimination) to a theoretical maximum of 1 (perfect discrimination).

The terms “treating” or “treatment” of a disease state or condition includes: (i) preventing the disease state or condition, i.e., causing the clinical symptoms of the disease state or condition not to develop in a subject that may be exposed to or predisposed to the disease state or condition, but does not yet experience or display symptoms of the disease state or condition, (ii) inhibiting the disease state or condition, i.e., arresting the development of the disease state or condition or its clinical symptoms, or (iii) relieving the disease state or condition, i.e., causing temporary or permanent regression of the disease state or condition or its clinical symptoms.

The term “sustained virologic response” (SVR) is used to describe a desired response in a patient when, e.g., hepatitis C virus is undetectable in the blood six months after finishing treatment. Conventional treatment using interferon and ribavirin doesn't necessarily eliminate, or clear, the hepatitis C virus. A sustained virologic response is associated with a very low incidence of relapse. SVR is used to evaluate new medicines and compare them with proven therapies.

The term “distinguishable cholate” or “distinguishable cholate compound” may be may any cholate compound that is distinguishable analytically from naturally occurring cholate in the blood or serum of a subject. The distinguishable cholate compound may be a labeled cholate compound or an unlabeled cholate compound. The distinguishable cholate compound may be a fluorescent moiety-labeled cholate compound. Various fluorescent probes are commercially available such as, e.g., fluorescein, Alexa Fluor dyes, quantum dots, and the like. The distinguishable cholate be an isotope labeled cholate compound. Distinguishable cholate compounds may be labeled with either stable isotopes (e.g., ¹³C, ²H, ¹⁸O) or radioactive isotopes (e.g., ¹⁴C, ³H). Distinguishable cholate compounds are commercially available and can be purchased (for example CDN Isotopes Inc., Quebec, CA).

The distinguishable cholate compounds may be stable isotope labeled cholate compounds. The distinguishable cholate may be selected from any known safe, non-radioactive stable isotope of cholic acid. In one specific aspect, the distinguishable cholate compound is 2,2,4,4-²H cholic acid, also known as cholic-acid-2,2,4,4-d₄ (D₄-CA). In another specific aspect, the distinguishable cholate compound is 24-¹³C cholic acid, also known as cholic acid-24-¹³C (¹³C-CA). In another specific aspect, the distinguishable compound is 2,2,3,4,4-²H cholic acid, also known as cholic acid-2,2,3,4,4-d₅ (D₅-CA).

In some embodiments, the distinguishable cholate compound may be selected from any of the following labeled compounds: cholic acid, any glycine conjugate of cholic acid, any taurine conjugate of cholic acid; chenodeoxycholic acid, any glycine conjugate of chenodeoxycholic acid, any taurine conjugate of chenodeoxycholic acid; deoxycholic acid, any glycine conjugate of deoxycholic acid, any taurine conjugate of deoxycholic acid; or lithocholic acid, or any glycine conjugate or taurine conjugate thereof. The distinguishable cholate compound may be selected from those described in WO 2021/207683 A1, HepQuant, LLC, Everson and Helmke, which is incorporated herein by reference in its entirety.

Cholates occur naturally and are not known to have any deleterious or adverse effects when given intravenously or orally in the doses used in the inventive or comparative tests herein. The serum cholate concentrations that are achieved by either the intravenous or oral doses are similar to the serum concentrations of bile acids that occur after the ingestion of a fatty meal. Because cholates are naturally occurring with a pool size in humans of 1 to 5 g, the 20 and 40 mg doses of labeled cholates used herein are unlikely to be harmful.

The term “oral cholate clearance” (Clₒᵣₐₗ) refers to clearance from the body of a subject of an orally administered cholate compound as measured by a blood or serum sample from the subject. Oral cholate clearance is used as a measure of portal blood flow. Orally administered cholic acid is absorbed across the epithelial lining cells of the small intestine, bound to albumin in the portal blood, and transported to the liver via the portal vein. Approximately 80% of cholic acid is extracted from the portal blood in its first pass through the liver. Cholic acid that escapes hepatic extraction exits the liver via hepatic veins that drain into the vena cava back to the heart, and is delivered to the systemic circulation. The area under the curve (AUC) of peripheral venous concentration versus time after oral administration of cholic acid quantifies the fraction of cholic acid escaping hepatic extraction and defines “oral cholate clearance”.

The term “portal hepatic filtration rate”, “portal HFR”, “FLOW test” (HFRp) refers to oral cholate clearance (portal hepatic filtration rate; portal HFR) used as a measure of portal blood flow, or portal circulation, obtained from analysis of concentration of distinguishable cholate compound in at least 5 blood samples drawn from a subject over a period of, for example, about 90 minutes after oral administration of a distinguishable cholate compound, for example, a distinguishable cholate. The units of portal HFR value are typically expressed as mL/min/kg, where kg refers to kg body weight of the subject. “Portal HFR”, mL min⁻¹ kg⁻¹ may be used to Model independent apparent clearance of orally administered d4-cholate, adjusted for body weight, and calculated from dose/AUC. FLOW test methods are disclosed in U.S. Pat. Nos. 8,778,299, 9,417,230, and 10,215,746, each of which is incorporated herein by reference in its entirety.

The term “Systemic HFR”, (HFRs) mL min⁻¹ kg⁻¹ may be used to Model independent clearance of intravenously injected 13C-cholate, adjusted for body weight, and calculated from dose/AUC. The “Systemic HFR”, mL min⁻¹ kg⁻¹, may be used to Model independent clearance of intravenously injected 13C-cholate, adjusted for body weight, and calculated from dose/AUC.

The term “STAT test” (STAT) refers to an estimate of portal blood flow by analysis from one patient blood sample drawn at a defined period of time following oral administration of a differentiable cholate. In one aspect, the STAT test refers to analysis of a single blood sample drawn at a specific time point after oral administration of a differentiable cholate. In one specific aspect, the STAT test is a simplified convenient test intended for screening purposes that can reasonably estimate the portal blood flow (estimated flow rate) from a single blood sample taken 60 minutes after orally administered deuterated-cholate. In some embodiments, STAT, is the d4-cholate concentration in the 60 minute blood sample. STAT correlates well with DSI and can be used to estimate DSI. The STAT test value is typically expressed as a concentration, for example, micromolar (uM) concentration. STAT test methods are disclosed in U.S. Pat. Nos. 8,961,925, 10,222,366, each of which is incorporated herein by reference in its entirety. STAT test value may be used to estimate portal HFR, as provided in U.S. Pat. Nos. 8,961,925, 10,222,366. A STAT test value in a patient may be used to estimate a DSI value in a patient, as provided herein.

The term “DSI test” (DSI) refers to Disease Severity Index test which is derived from one or more liver function test results based on hepatic blood flow. The DSI score is a function of the sum of cholate clearances from systemic and portal circulations adjusted to disease severity ranging from healthy subjects to end stage liver disease. DSI is a score without units representing a quantitative measurement of liver function. A disease severity index (DSI) value may be obtained in a patient by a method comprising (a) obtaining one or more liver function test values in a patient having or at risk of a chronic liver disease, wherein the one or more liver function test values are obtained from one or more liver function tests selected from the group consisting of SHUNT, portal hepatic filtration rate (portal HFR), and systemic hepatic filtration rate (systemic HFR); and (b) employing a disease severity index equation (DSI equation) to obtain a DSI value in the patient, wherein the DSI equation comprises one or more terms and a constant to obtain the DSI value, wherein at least one term of the DSI equation independently represents a liver function test value in the patient, or a mathematically transformed liver function test value in the patient from step; and the at least one term of the DSI equation is multiplied by a coefficient specific to the liver function test. DSI is an index, or score, that encompasses the cholate clearances from both systemic and portal circulations. DSI has a range from 0 (healthy) to 50 (severe end-stage disease) and is calculated from both HFRs. Based on the reproducibility of DSI values, the minimum detectable difference indicating a change in liver function in a subject may be about 1.5 points, about 2 points, or about 3 points. DSI test methods and equations are disclosed in U.S. Pat. Nos. 9,091,701, 9,759,731, 10,520,517, each of which is incorporated herein by reference in its entirety. A method of estimating a DSI value in a patient from a STAT test value is also provided herein.

The term “Hepatic Reserve” refers to percentage of maximum hepatic functional capacity measured by DSI, indexed hepatic reserve may be normalized to the DSI range in subjects of lean body mass. HR (algebraic) is simply an algebraic conversion of the DSI value in the subject: HR ([100−(2×DSI)]. Indexed HR is normalized against the results within a cohort of normal lean controls.

The term “RCA20” represents the amount of the intravenously administered distinguishable compound, for example, a distinguishable cholate compound such as 13C-CA, that remains in the circulation 20 minutes after the intravenous injection.

The term “Quantitative Liver Function Test” (QLFT), refers to assays that measure the liver's ability to metabolize or extract test compounds, can identify patients with impaired hepatic function at earlier stages of disease, and possibly define risk for cirrhosis, splenomegaly, and varices. One of these assays is the cholate shunt assay where the clearance of cholate is assessed by analyzing bodily fluid samples after exogenous cholate has been taken up by the body.

The term “Ishak Fibrosis Score” is used in reference to a scoring system that measures the degree of fibrosis (scarring) of the liver, which is caused by chronic necroinflammation. A score of 0 represents no fibrosis, and 6 is established fibrosis. Scores of 1 and 2 indicate mild degrees of portal fibrosis; stages 3 and 4 indicate moderate (bridging) fibrosis. A score of 5 indicates nodular formation and incomplete cirrhosis, and 6 is definite cirrhosis.

The term “Childs-Turcotte-Pugh (CTP) score” or “Child-Pugh score” refers to a classification system used to assess the prognosis of chronic liver disease as provided in Pugh et al., Transection of the oesophagus for bleeding oesophageal varices. Br J Surg 1973; 60:646-649, which is incorporated herein by reference. The CTP score includes five clinical measures of liver disease; each measure is scored 1-3, with 3 being the most severe derangement. The five scores are added to determine the CTP score. The five clinical measures include total bilirubin, serum albumin, prothrombin time international normalized ratio (PT INR), ascites, and hepatic encephalopathy. The CTP score is one scoring system used in stratifying the seriousness of end-stage liver disease. Chronic liver disease is classified into Child-Pugh class A to C, employing the added score. Child-Pugh class A refers to CTP score of 5-6. Child-Pugh class B refers to CTP score of 7-9. Child-Pugh class C refers to CTP score of 10-15. A website calculates post-operative mortality risk in patients with cirrhosis. http://mayoclinic.org/meld/mayomodel9.html

The term “Model for End-Stage Liver Disease (MELD) refers to a scoring system used to assess the severity of chronic liver disease. MELD was developed to predict death within three months of surgery in patients who had undergone a transjugular intrahepatic portosystemic shunt (TIPS) procedure patients for liver transplantation. MELD is also used to determine prognosis and prioritizing for receipt of a liver transplant. The MELD uses a patient's values for serum bilirubin, serum creatinine, and international normalized ratio for prothrombin time (INR) to predict survival. The scoring system is used by the United Network for Organ Sharing (UNOS) and Eurotransplant for prioritizing allocation of liver transplants instead of the older Child-Pugh score. See UNOS (2009 Jan. 28) “MELD/PELD calculator documentation”, which is incorporated herein by reference. For example, in interpreting the MELD score in hospitalized patients, the 3 month mortality is: 71.3% mortality for a MELD score of 40 or more.

The term “standard sample” refers to a sample with a known concentration of an analyte used for comparative purposes when analyzing a sample containing an unknown concentration of analyte.

The term “Chronic Hepatitis C” (CHC) refers to a chronic liver disease caused by viral infection and resulting in liver inflammation, damage to the liver and cirrhosis. Hepatitis C is an infection caused by a blood-borne virus that attacks the liver and leads to inflammation. Many people infected with hepatitis C virus (HCV) do not exhibit symptoms until liver damage appears, sometimes years later, during routine medical tests.

The term “Steatotic Liver Disease” (SLD) encompasses various etiologies of hepatic steatosis.

The term “Alcoholic SteatoHepatitis” (ASH) refers to a chronic condition of inflammation of the liver which is caused by excessive drinking. Progressive inflammatory liver injury is associated with long-term heavy intake of ethanol and may progress to cirrhosis.

The term “Metabolic dysfunction-Associated Steatohepatitis (MASH), formerly known as “Non-Alcoholic SteatoHepatitis” (NASH) refers to a serious chronic condition of liver inflammation, progressive from the less serious simple fatty liver condition called steatosis. Simple steatosis (alcoholic fatty liver) is an early and reversible consequence of excessive alcohol consumption. In people that don't drink much alcohol, the cause of fatty liver disease is less clear, but may be associated with factors such as obesity, high blood sugar, insulin resistance, or high levels of blood triglycerides. In certain cases the fat accumulation can be associated with inflammation and scarring in the liver. This more serious form of the disease is termed metabolic dysfunction-associated steatohepatitis (MASH), formerly known as non-alcoholic steatohepatitis (NASH). MASH is associated with a much higher risk of liver fibrosis and cirrhosis than MASLD. Patients with MASH have increased risk for hepatocellular carcinoma. MASLD may progress to MASH with fibrosis cirrhosis and hepatocellular carcinoma.

The term “Metabolic dysfunction-Associated Steatotic Liver Disease” (MASLD), formerly known as “Non-Alcoholic Fatty Liver Disease” (NAFLD) refers to a common chronic liver disease characterized in part by a fatty liver condition with associated risk factors of obesity, metabolic syndrome, and insulin resistance. Both MASLD and MASH are often associated with obesity, diabetes mellitus and asymptomatic elevations of serum ALT and gamma-GT. Ultrasound monitoring can suggest the presence of a fatty infiltration of the liver; differentiation between MASLD and MASH, typically requires a liver biopsy.

The term “Metabolic dysfunction-associated Alcoholic Liver Disease (Met-ALD), refers to MASLD patients that consume greater amounts of alcohol per week (>140 g/week females and >210 g/week males).

The term “Primary Sclerosing Cholangitis” (PSC) refers to a chronic liver disease caused by progressive inflammation and scarring of the bile ducts of the liver. Scarring of the bile ducts can block the flow of bile, causing cholestasis. The inflammation can lead to liver cirrhosis, liver failure and liver cancer. Chronic biliary obstruction causes portal tract fibrosis and ultimately biliary cirrhosis and liver failure. The definitive treatment is liver transplantation. Indications for transplantation include recurrent bacterial cholangitis, jaundice refractory to medical and endoscopic treatment, decompensated cirrhosis and complications of portal hypertension (PHTN). PSC progresses through chronic inflammation, fibrosis/cirrhosis, altered portal circulation, portal hypertension and portal-systemic shunting to varices-ascites and encephalopathy. Altered portal flow is an indication of clinical complications.

Any appropriate analysis method known in the art may be employed for quantification of the distinguishable cholate compounds in blood or serum samples. For example, detection and quantification of the distinguishable cholate compound in the sample may comprise high performance liquid chromatography (HPLC), HPLC-diode-array detection (HPLC-DAD), HPLC-fluorescence, ultra-performance liquid chromatography (UPLC), mass spectrometry (MS), GC-MS, LC-MS, LC-MS/MS, surface-enhanced Raman spectroscopy (SERS), immunoassays, for example, using isolated antibodies, monoclonal antibodies, or antigen-binding fragments thereof, single domain antibodies, aptamers, and the like. Methods for detection and quantification of distinguishable cholates are described in, for example, US20210318274, which is incorporated herein by reference in its entirety.

The blood or serum sample for use in the present methods may be collected from a subject by any known method in the art. For example, see WHO guidelines on drawing blood: best practices in phlebotomy, World Health Organization. 2010, Geneva, Switzerland or BP-EIA: Collecting, processing, and handling venous, capillary, and blood spot samples, PATH, 2005. For example, venipuncture using needle and syringe or indwelling catheter, arterial blood sampling, pediatric or neonatal blood sampling, or capillary sampling may be employed. The choice of site and procedure may depend on the volume of blood needed for the procedure and laboratory test to be done. For example, a venous site, finger-prick or heel-prick, also known as capillary sampling or skin puncture, may be employed.

Whole blood samples may be obtained by venipuncture, collected in anticoagulant-containing vacutainer tubes, and refrigerated during storage and shipment. Blood samples can be further processed into different fractions. The blood or serum samples may be peripheral blood samples. The sample may be a transcutaneous blood sample. Various commercial devices are available for obtaining transcutaneous samples such as, for example, single-use blood lancing devices intended for obtaining microliter capillary whole blood samples (e.g., Tasso, Inc., Seattle, WA).

Dried Blood Spots (DBS)

Dried blood spots (DBS) is a form of bio-sampling where blood samples are blotted and dried on filter paper. DBS may typically include the deposition of small volumes of capillary blood or venous blood onto dedicated paper cards. Comparatively to whole blood or plasma samples, their benefits rely in the fact that sample collection is easier and that logistic aspects related to sample storage and shipment can be relatively limited, respectively, without the need of a refrigerator or dry ice. Wagner et al., Mass Spectrometry Reviews, 2016, 35, 361-438.

DBS typically consist in the deposition of a few droplets of capillary blood, obtained by heel- or finger-pricking, onto filter papers in a card format (also known as “Guthrie cards”). Samples are simply allowed to dry, without any other processing. Chemically speaking, analytes are adsorbed with blood components onto a solid, cellulose-based matrix. Compared to conventional venipuncture, much less volume is required, blood collection is simple, noninvasive and inexpensive, risk of bacterial contamination or hemolysis is minimal, and DBS can be preserved for long periods of time with almost no deterioration of analytes allowing ease of transport due to sample stability. DBS sampling requires minimal sample volume, for example, about 10-100, 20-80, or 30-70 microliters per spot. DBS are therefore appropriate when the volume of blood collected is limited, for example in newborns, infants, or critically ill patients.

Paper cards dedicated to DBS are commercially available from several manufacturers and can be categorized in two groups: untreated and chemically treated papers. Untreated papers consist of pure cellulose and may be manufactured from 100% pure cotton linters. Treated papers may include cellulose treated with different proprietary chemicals. These may include Whatman (now part of GE Healthcare) FTA, FTA Elute, ETA DMPK-A, Whatman ETA DMPK-B (Majumdar & Howard, 2011), and Macherey Nagel NucleoCard (Moeller et al., 2012). FTA DMPK-A is impregnated with sodium dodecyl sulfate (SDS, <5%) and tris (hydroxymethyl)aminomethane (<5%), whereas FTA DMPK-B is impregnated with guanidinium thiocyanate (30-50%). Alternatively, untreated paper can be impregnated with chemicals by soaking it in a solution and allowing it to dry before use.

A blood collector card, dried blood spot (DBS) technology, or HemaSpot™ device, such as a HemaSpot™-HF device may be employed. For example, a HemaSpot™ HF device uses a finger-stick to collect and dry blood within a protective cartridge. For example, an EBF blood spot collection card, Eastern Business Forms, Inc. Mauldin, SC, may be employed, for example, a Five Spot blood card, or a Generic multipart card, wherein each circle holds up to about 75-80 microliters of sample. Once dried, the sample is stable at ambient temperature and can be safely and easily shipped to a laboratory for analysis.

Volumetric Absorptive Microsampling

Alternatively, a volumetric absorptive microsampling (VAMS™) device may be employed to obtain a blood sample. VAMS™ small volume collection devices are commercially available, for example, a Mitra® cartridge (Neoteryx, LLC). VAMS™ devices are handheld devices including a hydrophilic polymer tip connected to a plastic handle which wicks up a fixed volume (approximately 10, 20 or 30 microliters) when contacting a blood surface. VAMS effectively results in absorption of a fixed volume of blood, irrespective of the hematocrit.

Volumetric absorptive microsampling may take advantage of small volume sampling. A sample volume as low as, for example, 10, 20 or 30 microliters or more of a blood sample may be employed to wick a fixed volume of a capillary, venous blood, or serum sample.

Any appropriate form of extraction may be employed, as known in the art. Analysis may be performed according to any appropriate means, for example, LC-MS/MS.

DBS or VAMS blood samples may be eluted or extracted by any appropriate means. The DBS punch sample or a VAMS tip may be exposed to an extraction solution to solubilize the analyte. The punch sample or VAMS tip may be optionally presoaked in water. The extraction solution may be, for example, water, acetonitrile, methanol, methanol-acetonitrile, methanol-water-formic acid, methanol-water, (e.g., 90% aq. MeOH; 4:1 v/v), or CHCl₃/MeOH (e.g., 2:1 v/v), for example, at ambient room temperature of about 25° C., without stirring for 30 min. or more. Optionally, the punch samples in the extraction solution may be vortexed, sonicated, incubated, and centrifuged. The supernatant may be dried in a lyophilizer. The dried sample may be dissolved in, or extracted using an extraction solution and diluted in, a mobile phase buffer (e.g., acetonitrile-water-formic acid; e.g., 5:95:0.1, v/v) and transferred to sample vials or multi-well format for any appropriate analysis method, for example, comprising LC-MS/MS.

The following abbreviations are employed in the present disclosure. 13C-CA=carbon-13-labeled cholate; AIC=Akaike Information Criterion; BMI=body mass index; Cl confidence interval; CLD=chronic liver disease; CM=compartmental model; d4-CA=deuterium-labeled cholate; DSI=disease severity index; ER=extraction ratio; HCV=hepatitis C virus; HFR=hepatic filtration rate; HR=hepatic reserve; ICC=intraclass correlation coefficient; IV=intravenous; LC-MS=liquid chromatography-mass spectrometry; LC-MS/MS=liquid chromatography-tandem mass spectrometry. MM=minimal model; MMvd-=minimal model based on volume of distribution; MSE=mean squared error; NAFLD=nonalcoholic fatty liver disease; NASH=non-alcoholic steatohepatitis; SLD=steatotic liver disease; MetALD=metabolic dysfunction-associated alcoholic liver disease; MASLD=metabolic dysfunction-associated steatotic liver disease; MASH=metabolic dysfunction-associated steatohepatitis; TBV=total blood volume.

### Compartmental Modeling Versus Non-Compartmental Analysis

There are two main approaches that may be used to describe the pharmacokinetics (PK) of administered compounds: compartmental models and noncompartmental analysis. Compartmental models divide the body into a series of one or more, two or more, or three or more, compartments of different volumes and are described by a series of kinetic equations simulating the transfer of flow from one compartment to another. Compartmental models assume that each of the compartments is kinetically homogeneous and that the drug is instantaneously and evenly distributed throughout the compartment. The mathematics of compartmental modeling typically involve systems of first-order ordinary differential equations with constant coefficients. While some compartmental models are descriptive and achieve adequate fits to clearance data using two or three compartments, more realistic models which attempt to define underlying physiological mechanisms can be developed using multi-compartmental systems. On the other hand, noncompartmental analysis, as the name implies, does not use assumptions about body compartments and is regarded as model independent. Noncompartmental analysis often uses relatively simple algebraic equations to estimate summary PK parameters. As a result, noncompartmental methods require fewer assumptions than model-based approaches and are generally simpler, faster, and cheaper to develop relative to compartmental models.

Previously, a noncompartmental analysis, hereafter referred as the minimal model (MM), was used to characterize intravenous (IV) clearance by exponential fits and oral clearance by a cubic spline fit (Everson, G. T., et al., Portal-systemic shunting in patients with fibrosis or cirrhosis due to chronic hepatitis C: the minimal model for measuring cholate clearances and shunt. Alimentary Pharmacology & Therapeutics, 2007. 26(3): p. 401-410). More recently, the present inventors developed a compartmental model of the cholate SHUNT test which allowed determination of anatomic shunting and hepatic extraction, as well as improved the within individual reproducibility of SHUNT test measurements (McRae, M. P., et al., Compartmental model describing the physiological basis for the HepQuant SHUNT test. Translational Research).

The present disclosure uses both compartmental and noncompartmental methods to estimate systemic and portal clearances from a reduced set of inputs (i.e., DuO and TRIO) and compares the results to previous cholate SHUNT liver function test measurements.

### Artificial Intelligence (AI) Methods for Clearance Function Fitting

An alternative approach to traditional pharmacokinetic analyses is to train AI algorithms to perform the clearance curve fitting. AI methods, such as neural networks, can generalize nonlinear relationships between inputs and outputs. Such a tool could be trained with SHUNT test data (i.e., data collected with sampling resolution of 5 oral and 5 intravenous timepoints) to approximate the liver's portal and systemic clearance functions (i.e., MM curves) using only a limited number of inputs (e.g., 2 oral timepoints and 1 intravenous timepoint). This current work investigates the feasibility of AI DuO and AI TRIO, which are AI-based function approximation and nonlinear regression methods for estimating systemic and portal clearances from a reduced set of inputs.

### DuO Cholate Liver Function Test Development

A compartmental model (CM) was developed to fit portal clearance curves from DuO cholate tests. Compartmental models are defined by distribution volumes and transfer rates between volumes to estimate parameters not defined by noncompartmental analyses. The CM describes transfer of cholates between systemic, portal, and liver compartments with assumptions from measured or literature-derived values and unknown parameters estimated by nonlinear least-squares regression. Two versions of the DuO cholate test have been developed.

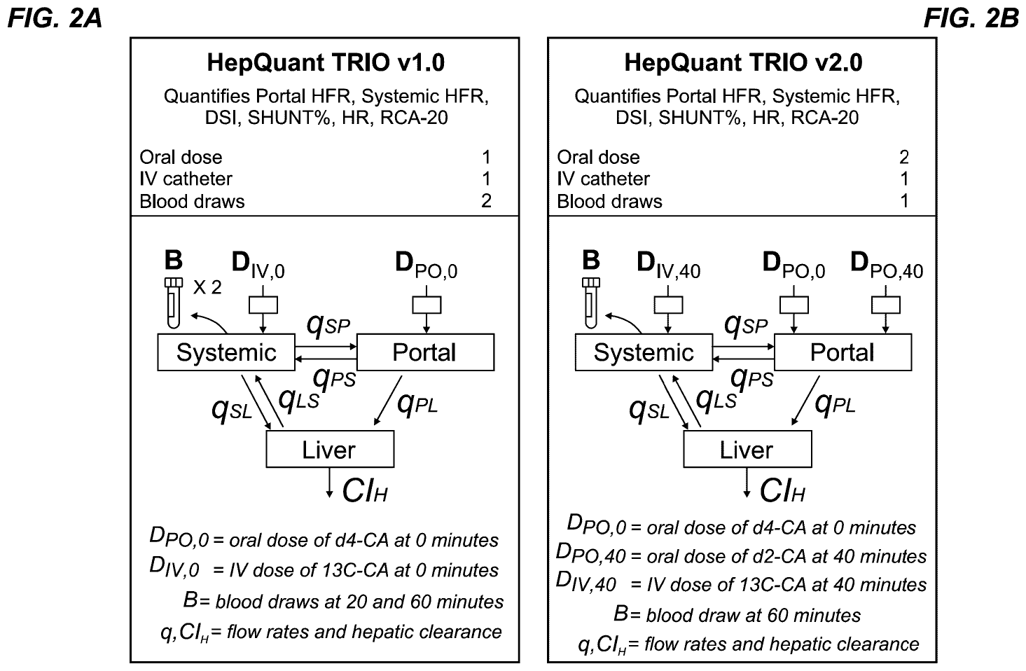

DuO cholate tests estimate portal clearance using either 1 oral cholate dose and 2 blood draws (DuO v1.0, FIG. 1A) or 2 oral cholate doses and 1 blood draw (DuO v2.0, FIG. 1B). Both DuO test versions comprise obtaining two orally administered distinguishable cholate concentration timepoints. Both DuO test versions involve the same analysis method.

DuO Compartmental Model Description

The flow between systemic (S), portal (P), and liver (L) compartments was described by a system of first-order ordinary differential equations (Equations 1A-3) where q is the flow rate between compartments, V is the volume of the compartment, Cis the concentration of cholate in the compartment, and Cl_{H} is the hepatic clearance.

$$\frac{{dC}_{S}}{dt} = \frac{1}{{V}_{S}} ( - [ {q}_{SP} + {q}_{SL} ] {C}_{S} + {q}_{LS} \cdot {C}_{L} ) . \qquad Eqn . 1 A$$

$$\frac{{dC}_{P}}{dt} = \frac{1}{{V}_{P}} ( {D}_{PO} + {q}_{SP} \cdot {C}_{S} - {q}_{PL} \cdot {C}_{P} ) . \qquad Eqn . 2 A$$

$$\frac{{dC}_{L}}{dt} = \frac{1}{{V}_{L}} ( - [ {Cl}_{H} \cdot {V}_{L} + {q}_{LS} ] {C}_{L} + {q}_{PL} \cdot {C}_{P} + {q}_{SL} \cdot {C}_{S} ) . \qquad Eqn . 3$$

The following sections describe various aspects of the compartmental model.

Compartmental Volumes

Total blood volume (TBV) was calculated by Equation 4 which accounts for the nonlinear relationship between blood volume and body mass index (BMI, kg/m²) across the entire range of body weights (BW) including obese (BMI 30-40) and morbidly obese (BMI>40) subjects [8].

$$TBV = \frac{0.07 \cdot BW}{\sqrt{BMI / 22}} . \qquad Eqn . 4$$

In this equation, 22 is the BMI value corresponding to ideal body weight, and 0.07 is the indexed blood volume (in L·kg⁻¹) for a subject with a BMI of 22.

Whole blood is comprised of approximately 55% plasma and 45% hematocrit [9]. Since the SHUNT test uses serum sampling, any cholate distributed to hematocrit is not measured. A plasma fraction (fₚₗₐₛₘₐ) was used for adjusting volumes according to the fraction of whole blood without red blood cells (i.e., 1 minus hematocrit) to approximate the serum sampling used in the cholate SHUNT test. The systemic compartment volume (V_{S}) is equal to the total plasma volume which is estimated by multiplying TBV by fₚₗₐₛₘₐ as shown in equation 4A. It is the systemic compartment which represents the total plasma volume from which measurements of cholate are derived.

V_{S}=TBV·(1−Hct) Eqn. 4A.

It is estimated that the liver receives 25% of the total cardiac output [10, 11], and the splanchnic organs contain 25% of total blood volume at rest [12]; thus, the volume of the hypothetical portal compartment (V_{P}) was set to 25% of the systemic volume, as shown in equation 4B.

V_{P}=0.25·V_{S} Eqn. 4B.

To calculate the volume of the liver compartment (V_{L}), liver volumes corrected for body weight from 13 studies of ultrasound and computerized tomography scans [13] were averaged (22.46±1.98 mL·kg⁻¹) and multiplied by BW and liver tissue density (d_{L}) to obtain liver weight (w_{L}). Liver tissue density of 1.06 g·mL¹ was derived from the average of two studies estimating liver densities of 1.04 g·mL⁻¹ [14] and 1.08 g·mL⁻¹ [15]. Hepatic blood volume ranges from 25 to 30 mL per 100 g liver weight [10, 16]. Using the average of this range (0.275 mL·g⁻¹), the volume of the liver compartment, V_{L}, was estimated by Equation 5A.

V_{L}=(0.275·22.46·BW·d_{L}fₚₗₐₛₘₐ)/1000 Eqn. 5A.

Flow Parameters

A system of differential equations describes the 13C-CA and d4-CA concentrations in the systemic (C_{S}), portal (C_{P}), and liver (C_{L}) compartments (Equations 1-3). Total hepatic flow rate (Q_{L}) was comprised of both hepatic arterial (q_{SL}) and portal venous (q_{PL}) inflows to the liver (Equation 6A).

Q_{L}=q_{SL}+q_{PL} Eqn. 6A.

Using the previous estimate for liver weight, w_{L}, the initial estimate for total hepatic flow rate (Q_{L, init}) was approximately 1 L·min⁻¹·kg⁻¹ liver wet weight [10, 17].

Splanchnic circulation (q_{SP}) is defined as the blood flow to the abdominal gastrointestinal organs, including the stomach, liver, spleen, pancreas, and intestines. A variety of mechanisms for splanchnic blood flow control have been proposed in which the splanchnic vascular bed functions with an autoregulatory capacity to maintain a constant blood flow across a range of perfusion pressures [18]. For the purpose of this model, q_{SP} was assumed to be proportional to the total cardiac output. Since splanchnic circulation accounts for approximately 75% of the total liver inflow (Q_{L}) [10], q_{SP} was defined as a constant flow rate at 75% of Q_{L}, where Q_{L} is 25% of the total cardiac output.

The liver's unique dual blood supply draws from both the hepatic artery and the portal vein. Hepatic arterial inflow (q_{SL}) accounts for about 25% of the total inflow to the liver [10], is highly adaptable [19], and is capable of compensating for changes in portal venous flow with studies suggesting that 25%-60% of decreased portal flow can be buffered by the hepatic artery [20, 21]. For model simplification, q_{SL} was defined as 25% of Q_{L}, where Q_{L} is 25% of the total cardiac output.

In health, the portal venous inflow to the liver (q_{PL}) equals the splanchnic arterial flow rate (q_{SP}). However, in the presence of collateral circulation (e.g., portosystemic shunts or esophageal and gastric varices), portal venous inflow is reduced by the shunt flow (q_{PS}) which bypasses the liver. To simplify the model for the purpose of fitting generalizable oral clearance curves to limited data points, the shunt flow rate was not estimated (i.e., q_{PS} was eliminated from the model). Instead, the effect of shunting was captured in the estimation of a scaling factor (F) that is related to the absolute bioavailability and described in more detail in the Oral Dose Administration section below.

Lastly, the total hepatic venous return flow to systemic circulation (q_{LS}) is equal to the total inflow to the liver, Q_{L}.

Hepatic Extraction

Hepatic extraction ratio (ER) is defined as the fraction of drug entering the liver which is irreversibly removed during a single pass through the liver [22, 23]. Drugs with high ER are rapidly cleared by the liver, whereby the clearance depends primarily on hepatic blood flow. The hepatic uptake of bile acids is exceptionally efficient with extraction ratios of 50-90% depending on the bile acid structure [24]. Gilmore and Thompson studied the clearance of cholate in 14 human controls and found mean (standard deviation) extraction ratios of 77.0% (7.5%) [25]. Similarly, O'Maille, Richards, and Short measured an extraction ratio of 79.0% (8.0%) in dogs [26]. For cholate, an extraction ratio greater than 0.7 is considered high [27]; thus, clearance is primarily driven by flow to the liver. Since the concentration of cholate in systemic venous plasma is relatively low, and because it's extensively bound to albumin, the amount of cholate eliminated in urine is negligible [24] and, therefore, it was assumed that the administered cholate was eliminated entirely by the liver.

Free cholate is eliminated from the liver with total hepatic clearance defined as the hepatic inflow times the extraction ratio [22, 23]. Due to the high extraction ratio of cholate (ER>0.7) and relatively constant intrinsic hepatocyte clearance across the spectrum of liver disease, differences in cholate clearance in CLD are assumed to be primarily attributed to altered flow to the liver. In the DuO compartmental model, hepatic clearance, Clin, may be calculated according to Equation 7A.

Cl_{H}=Q_{L}·ER Eqn. 7A.

Here, ER was determined through parameter estimation with an initial estimate of 0.7.

Effects of Albumin

It is important to note that the DuO cholate test is measuring the flow-dependent and highly efficient uptake of cholate by the liver and not metabolism by the liver cells. The hepatic uptake of organic anions has been previously described according to the conventional “free-drug” hypothesis in which the concentration of free ligand controls the hepatic uptake rate [28, 29]. However, despite their highly efficient hepatic uptake, organic anions have a strong affinity for binding to serum albumin. Alternative “albumin-mediated” uptake models may explain cholate's highly efficient uptake by the liver [30, 31].

In the case of TRIO cholate test, the intravenously administered distinguishable cholate (e.g., 13C-CA) is pre-bound to albumin prior to administration to deter binding to cells/tissues, ensure its residency in the intravascular space, and facilitate efficient uptake by hepatocytes. For the DuO cholate test's oral distinguishable cholate (e.g., d4-CA) dose, it is assumed that the orally administered distinguishable cholate (e.g., d4-CA) is extensively bound to albumin upon intestinal absorption. While a fraction of the orally administered distinguishable cholate (e.g., d4-CA) dose may be exposed to binding by red blood cells and extravascular tissues, for simplification of the compartmental model the administered dose was assumed to enter and remain completely within the intravascular space and bound to albumin for the duration of the test until hepatic extraction.

Oral Dose Administration

Oral dose administration was modeled via a flexible transit model [32, 33] which has been demonstrated to closely describe absorption delay observed in oral drug administration. A transit model was adapted to describe the passage of oral dose (D_{PO}) through a series of n non-integer hypothetical transit compartments to simulate drug absorption delay and account for first-pass extraction. Equation 8A can be used to describe the rate of change of the amount of d4-CA (dA_{d4}/dt) entering systemic circulation.

$$\frac{{dA}_{PO}}{dt} = {D}_{PO} \cdot F \cdot {k}_{TR} \cdot \frac{{( {k}_{TR} \cdot t )}^{n} \cdot {e}^{- {k}_{TR} \cdot t}}{\sqrt{2 π} \cdot {n}^{n + 0.5} \cdot {e}^{- n}} , \qquad Eqn . 8 A$$

$$wherein$$

$${k}_{T R} = \frac{n + 1}{M T T} . \qquad Eqn . 9 A$$

Here, t is time in minutes; k_{TR} is the transit rate constant between compartments (Equation 9A); MTT is an estimated parameter representing the mean transit time of a d4-CA molecule through intestinal absorption and into systemic circulation (initial estimate of 30 minutes); F is an estimated parameter that scales the oral clearance curve and is related to the first-pass bioavailability (initial estimate of 0.20).

DuO Method of Calculating Hepatic Disease Indices

The following metrics are measured by the DuO cholate liver function test and have previously demonstrated associations with liver function:
Area under the oral curve (AUC_{Oral}) is measured by first simulating the full oral clearance curve using the compartmental model and calculating the area using the trapezoidal numerical integration.
Portal hepatic filtration rate (HFR_{P}) is the portal clearance adjusted for subject body weight (BW) calculated by Equation 10A.

$$H F {R}_{P} = \frac{{D}_{P O}}{{AUC}_{Oral} \cdot BW} . \qquad Eqn . 10 A$$

The following metrics are estimated by the cholate DuO test and have previously demonstrated associations with liver function.
Area under the IV curve (AUC_{IV}) is estimated via a linear regression model (Equation 11A).

AUC_{IV}=β₀+β_{BW}·BW+β_{PO,20}·C_{PO,20}+β_{PO,60}·C_{PO,60}+β_{HFR,P}·HFR_{P} Eqn. 11A.

This linear model was trained using AUCiv data from SHUNT-V and resulted in the regression coefficients listed in Table 1B. To protect against overfitting, the linear model was trained using a 5-fold cross-validation procedure by partitioning the data into folds and estimating accuracy on each fold. Predictors considered in the model included those listed in Table 1B in addition to AUC_{Oral} and V_{d}. The best performing model and subset of model parameters which minimized the root mean square error (RMSE) between the predicted AUC_{IV} and the AUC_{IV} calculated from the Minimal Model was selected.

TABLE 2B
Linear regression model coefficients for estimating
AUC_{IV} from cholate DuO test data

| Coefficient | Description | Value |
|---|---|---|
| β₀ | Intercept | 161.9720 |
| B_{BW} | Body weight | −0.6459 |
| β_{PO, 20} | Concentration of d4-CA at 20 min | 16.9249 |
| β_{PO, 60} | Concentration of d4-CA at 60 min | 89.2405 |
| β_{HFR, P} | Portal HFR measured by DuO | −0.4755 |

Systemic hepatic filtration rate (HFR_{S}) is the estimated systemic clearance adjusted for body weight (Equation 12). Here, the IV dose (D_{IV}) is assumed to be half of the administered oral dose amount.

$${HFR}_{S} = \frac{{D}_{IV}}{{AUC}_{IV} \cdot BW} . \qquad Eqn . 12$$

SHUNT is the estimated absolute bioavailability of orally administered distinguishable cholate (e.g., d4-CA) (Equation 13).

$$F = \frac{{AUC}_{Oral} \cdot {D}_{IV}}{{AUC}_{IV} \cdot {D}_{PO}} . \qquad Eqn . 13$$

DSI is a score indicative of overall liver function comprising both portal and systemic HFR. The DSI is intended to convey unique information regarding fibrosis stage and clinical stages of cirrhosis, and modeling these outputs for prediction of clinical outcomes produced the DSI in equation 14 [6, 34-36]. Here, HFR_{P,max} and HFR_{S,max} are the upper limits of clearance for healthy controls, and A is a factor to scale DSI from 0 to 50.

$$DSI = A \cdot \sqrt{{( ln [ \frac{H F {R}_{P , max}}{H F {R}_{P}} ] )}^{2} + {( ln [ \frac{{HFR}_{S , max}}{{HFR}_{S}} ] )}^{2}} . \qquad Eqn . 14$$

Indexed Hepatic Reserve (HR_{indexed}) is a numerical index representing overall hepatic health with values between 0 and 100 (Equation 15). Here, HR_{indexed} uses HFR_{P} and HFR_{S} indexed to lean controls minus one standard deviation (HFR_{P,lean} and HFR_{S,lean}) with constant A to scale HR_{indexed} from 100 to 0.

$${HR}_{indexed} = 100 - A \cdot \sqrt{{( ln [ \frac{H F {R}_{P , leαn}}{H F {R}_{P}} ] )}^{2} + {( ln [ \frac{{HFR}_{S , lean}}{{HFR}_{S}} ] )}^{2}} . \qquad Eqn . 15$$

Algebraic Hepatic Reserve (HR_{algebraic}) is a numerical index representing overall hepatic health with values between 0 and 50 (Equation 15A).

HR_{algebraic}=100−2·DSI Eqn. 15A.

TRIO Cholate Liver Function Test Development

The TRIO cholate test is similar to the DuO cholate test, except with the addition of a single IV-administered dose of distinguishable cholate (e.g., 13C-CA) and its measurement at 20 minutes. The addition of this data point allows for more accurate estimation of the systemic clearance curve, allowing for quantification of test parameters that require accurate measurements of both AUC_{Oral} and AUC_{IV}, such as systemic HFR, DSI, SHUNT, and HR.

TRIO tests quantify portal HFR, Systemic HFR, DSI, SHUNT %, HR, and RCA-20.

Two versions of the TRIO cholate liver function test have been developed. The TRIO tests estimate both portal and systemic clearances using either 1 oral+1 IV dose+2 blood draws (TRIO V1.0, FIG. 2A, also known as SHUNT 2.0) or 2 oral doses+1 IV dose+1 blood draw (TRIO V2.0, FIG. 2B, also known as SHUNT 3.0). Both versions of the TRIO test use the same analysis method.

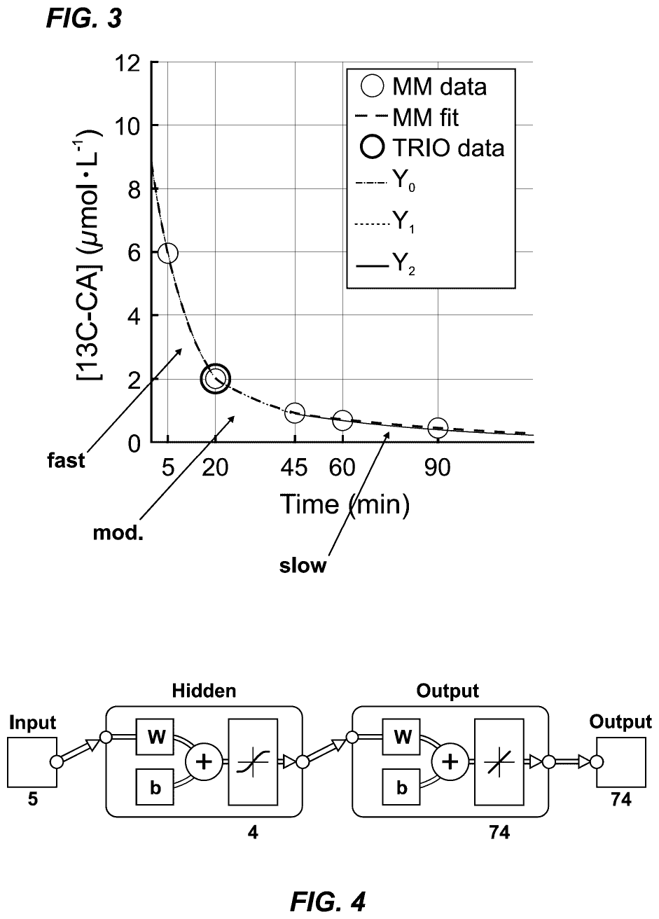

A noncompartmental analysis method was developed involving the exponential fits of systemic cholate clearance using only the 20 min. intravenous distinguishable (e.g., 13C-CA) concentration timepoint. The exponential fits split the curve into three sections or phases: fast, moderate, and slow (0 to 20 min., 20-45 min., and 45-180 min. for Y₀, Y₁, and Y₂, respectively) (FIG. 3).

TRIO Analysis Description

Fast Phase

The first phase of clearance, between 0 and 20 minutes, represents a fast distribution phase. To estimate the initial 13C-CA concentration, the volume of distribution (V_{d}) in L per kg body weight was first calculated by Equation 16A which accounts for the nonlinear relationship between blood volume and body mass index (BMI, kg/m²) across the entire range of body weights including obese (BMI 30-40) and morbidly obese (BMI>40) subjects [8].

$$TPV = {V}_{d} = \frac{0.07}{\sqrt{BMI / 22}} \cdot ( 1 - Hct ) , \qquad Eqn . 16 A$$

wherein PV is total plasma volume, BMI is body mass index, and Hct is hematocrit in the subject.

In this equation, 22 is the BMI value corresponding to ideal body weight, and 0.07 is the indexed blood volume (in L·kg⁻¹) for a subject with a BMI of 22. The initial 13C-CA concentration after dose administration was then estimated by dividing the dose by V_{d} times body weight (BW) (Equation 17).

$${C}_{0} = \frac{{D}_{IV}}{{V}_{d} \cdot BW} . \qquad Eqn . 17$$

Finally, the rate of elimination in the fast phase is defined by k_{fast} (Equation 18).

$${k}_{fast} = \frac{ln ( {C}_{0} ) - ln ( {C}_{20} )}{20} . \qquad Eqn . 18$$

Moderate Phase

The second phase of clearance, between 20 and 45 minutes, represents a moderate elimination phase in which the elimination rate k_{mod} was estimated using a linear model (Equation 19).

k_{mod}=β_{0,mod}+β_{kfast}·k_{fast}+β_{IV,20}·C_{IV,20}+β_{PO,20,mod}·C_{PO,20}+β_{PO,60,mod}·C_{PO,60} Eqn. 19.

This linear model was trained using systemic clearance data from the SHUNT-V study and resulted in the regression coefficients listed in Table 2. To protect against overfitting, the linear model was trained using a 5-fold cross-validation procedure by partitioning the data into folds and estimating accuracy on each fold. Predictors considered in the model included those listed in Table 2 in addition to body weight and IV dose. The best performing model and subset of model parameters which minimized the root mean square error (RMSE) between the predicted k_{mod} and the k_{mod} calculated from the Minimal Model was selected.

TABLE 2
Linear regression coefficients for estimating k_{mod}.

| Coefficient | Description | Value |
|---|---|---|
| β_{0, mod} | Intercept | 0.0268 |
| β_{k, fast} | k_{fast} | 0.0546 |
| β_{IV, 20} | Concentration of 13C-CA at 20 min | 0.0045 |
| β_{PO, 20, mod} | Concentration of d4-CA at 20 min | −0.0007 |
| β_{PO, 60, mod} | Concentration of d4-CA at 60 min | −0.0052 |

Slow Phase

The final phase of clearance, at times greater than 45 minutes, represents a slow elimination phase in which the elimination rate k_{low} was set to the mean value for SHUNT-V subjects, which was 0.018 min⁻¹.

Exponential Fits

The exponential equations defining the systemic concentration of 13C-CA through time are as follows:

$${Y}_{0} = {C}_{0} \cdot {e}^{- {k}_{fast} \cdot t} ; \qquad Eqn . 20$$

$${Y}_{1} = \frac{{C}_{20}}{{e}^{- {k}_{mod} \cdot 20}} \cdot {e}^{- {k}_{mod} \cdot t} ; and \qquad Eqn . 21$$

$${Y}_{2} = \frac{{C}_{45}}{{e}^{- {k}_{slow} \cdot 45}} \cdot {e}^{- {k}_{slow} \cdot t} . \qquad Eqn . 22$$

Here, t is time (0 to 20 min., 20-45 min., and 45-180 min. for Y₀, Y₁, and Y₂, respectively), C₀ is the initial concentration of 13C-CA, C₂₀ is the measured 20-minute concentration of 13C-CA, and C₄₅ is the estimated 45-minute concentration of 13C-CA. The areas under each of the three exponential curve fits are calculated by trapezoidal numerical integration and summed to estimate the AUC_{IV}.

TRIO Method of Calculating Disease Indices

The following metrics are measured by the cholate TRIO test and have previously demonstrated associations with liver function:
Area under the oral curve (AUC_{Oral})—same as DuO
Portal hepatic filtration rate (HFR_{P})—same as DuO
Area under the IV curve (AUC_{IV}) is measured by trapezoidal numerical integration of the exponential fits from TRIO.
Systemic hepatic filtration rate (HFR_{S}) is the measured systemic clearance adjusted for body weight using the same equation as DuO (Equation 12), however, the IV dose (D_{IV}) is the actual administered amount and AUC_{IV} is measured as described above.
SHUNT is calculated using the same equation as DuO (Equation 13), but with instead using the measured AUC_{IV} value.
DSI is calculated using the same equation as DuO (Equation 14), but with instead using the measured AUC_{IV} value.
Hepatic Reserve (HR) is calculated using the same equation as DuO (Equation 15), but with instead using the measured AUC_{IV} value.

Cholate AI Test Methods

Artificial intelligence (AI), achieved using methods such as neural networks, is capable of learning and fitting complex functions with a limited number of inputs. Function fitting is the process of training a neural network on a set of inputs to produce an associated set of target outputs. Here, a neural network is constructed with the desired network topology and learning algorithm, and it is trained using a set of training data. After the network has fit the data, it forms a generalization of the input-output relationship. This trained network is then used to infer outputs for future data not used in training.

In this analysis, function fitting neural networks were used to predict the full oral and IV clearance curves using data from the cholate DuO and TRIO tests (i.e., Cholate AI DuO and AI TRIO). While this analysis focuses on the application of function fitting neural networks, similar results could be achieved with alternative methods for function approximation and nonlinear regression, including nonlinear nonparametric statistics (NNS), deep learning (i.e., deep neural networks), generative adversarial networks (GAN), random forest regression, ensemble methods, and/or any combination of interpolation, extrapolation, nonlinear regression, and curve fitting methods.

The AI analysis may comprise using function fitting neural networks to predict full oral and IV clearance curves using data from the DuO and TRIO liver function tests provided herein.

Cholate AI DuO Test Method

The objective of the AI DuO method is to predict both oral and IV clearance curves using only orally administered distinguishable cholate (e.g., d4-CA) clearance data from the DuO test. The full list of inputs to the neural network comprises a 5×1 array of:
(i.) 20-min. orally administered cholate (e.g., d4-CA) concentration;
(ii.) 60-min. orally administered cholate (e.g., d4-CA or d5-CA) concentration;
(iii.) Estimated V_{d} (Equation 16A);
(iv.) BMI; and
(v.) Hematocrit.

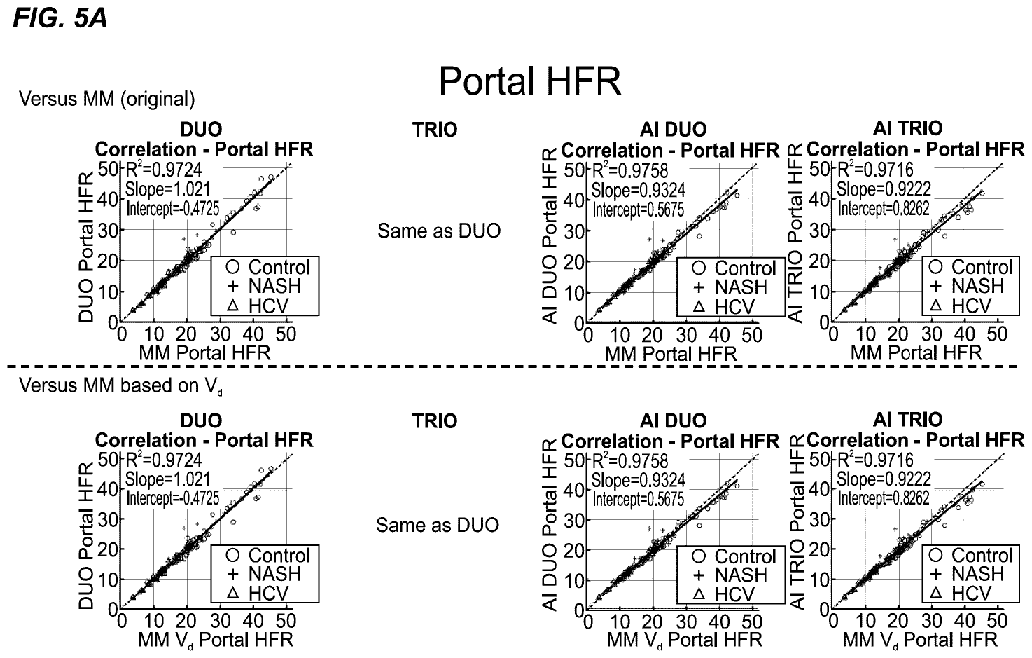

The neural network outputs selected points from the oral and IV curves estimated by the MM (i.e., 5-minute increments from 0 to 180 minutes, resulting in 37 timepoints for both oral and IV curves, rearranged into a 74×1 array). The network was configured for regression tasks and consists of a 2-layer feedforward network with a sigmoid transfer function in the hidden layer and a linear transfer function in the output layer (FIG. 4). The hidden layer size was optimized for performance (mean squared error) on a test set.

The training, validation, and test data consisted of a total of 542 subjects (N=217 from the HALT-C study, N=275 from the SHUNT-V study, and N=50 healthy with lean and overweight body mass index. Training data was split into training (70%), validation (15%), and test sets (15%). The training algorithm was Levenberg-Marquardt backpropagation; however, many alternative training algorithms could be used (Bayesian Regularization, BFGS Quasi-Newton, Resilient Backpropagation, Scaled Conjugate Gradient, Conjugate Gradient with Powell/Beale Restarts, Fletcher-Power Conjugate Gradient, Polak-Ribiére Conjugate Gradient, One Step Secant, Variable Learning Rate Gradient Descent, Gradient Descent with Momentum, Gradient Descent, etc. [Hagan, M. T., H. B. Demuth, and M. H. Beale, Neural Network Design, Boston, MA: PWS Publishing, 1996, Chapters 11 and 12]). The performance of the model was externally validated using data from a reproducibility study (see Analysis of Reproducibility section below for results).

Cholate AI TRIO Test Method

The objective of the AI TRIO method is the same as AI DuO—to predict both oral and IV clearance curves; however, it uses two oral d4-CA and one 13C-CA measurements from the TRIO test. The full list of inputs to the neural network comprises a 5×1 array of:
(i.) 20-min. oral distinguishable cholate (e.g., d4-CA) concentration;
(ii.) 60-min. oral distinguishable cholate (e.g., d4-CA or d5-CA) concentration;
(iii.) 20-min intravenous distinguishable cholate (e.g., 13C-CA) concentration;
(iv.) Estimated V_{d} (Equation 16A); and
(v.) Estimated initial concentration of intravenous distinguishable cholate (e.g., 13C-CA) based on V_{d}.

The AI TRIO network has the same architecture (FIG. 4) and training methods as AI DuO.

Cholate AI Method of Calculating Hepatic Disease Indices

Cholate tests AI DuO and AI TRIO analyses generate both oral and IV clearance curves. The AUC_{IV} and AUC_{Oral} are measured by trapezoidal numerical integration, and the remaining test parameters are calculated: HFR_{P} (Equation 10A); HFR_{S} (Equation 12); SHUNT (Equation 13); DSI (Equation 14); and HR (Equation 15).

Minimal Model Based on Volume of Distribution (MM_{Vd})

Previously, a noncompartmental analysis of the cholate SHUNT liver function test, hereafter referred as the minimal model (MM), was used to characterize intravenous (IV) clearance by exponential fits and oral clearance by a cubic spline fit (Everson et al., Alimentary Pharmacology & Therapeutics, 2007. 26(3): p. 401-410). This method used the slope of the 5-minute and 20-minute IV log-linear regression to estimate the concentration of 13C-CA at 0 minutes.

The initial clearance of IV-administered 13C-CA from systemic circulation is a rapid exponential decrease in concentration. Thus, estimating the initial concentration based on the 5-minute timepoint is potentially sensitive to variability in the test administration (e.g., the timing of the dose administration and 5-minute blood sample collection, one- versus two-arm catheter administration/collection, residual cholate in the catheter line, etc.). In other words, small variations in the measurement and timing of the 5-minute 13C-CA concentration would lead to significant changes in the area under the IV curve (AUC_{IV}), and thus, the cholate test parameters which depend on it (Systemic HFR, SHUNT, DSI, HR).

A new version of cholate Minimal Model test method analysis minimal model based on volume of distribution (MM_{Vd} analysis method, SHUNT V1.1 test) is provided herein in which the initial intravenous distinguishable cholate (e.g., 13C-CA) concentration at 0 minutes is calculated using physical characteristics of the subject (body weight and BMI) as opposed to estimating based on the slope of the 5-minute and 20-minute IV log-linear regression. Here, V_{d} is first estimated by Equation 16A, then the initial concentration is estimated by dividing the IV dose by V_{d} times body weight. This estimated initial concentration value is then used in the original MM (SHUNT V1.0) equations to construct the IV clearance curve.

Analysis of Reproducibility

Data

Data from a previous study of cholate test reproducibility were analyzed retrospectively. The study consisted of 48 subjects from three groups: controls (N=16), NASH (N=16), and HCV (N=16). Controls were healthy persons with no history of liver disease and normal standard blood tests. NASH was diagnosed based on risk factors (obesity, diabetes, metabolic syndrome), negative tests for other liver disease, and fibrosis stage by liver biopsy or transient elastography. HCV was diagnosed based on a history of positive HCV by nucleic acid testing and METAVIR fibrosis stage by liver biopsy. Three replicate cholate SHUNT tests were conducted on three separate days within 30 days. The original study provides further details regarding recruitment and test administration (Burton, J. R., et al., Translational Research, 2021. 233: p. 5-15).

Statistical Analysis

Test parameters were measured or estimated for all six methods (DuO, TRIO, AI DuO, AI TRIO, MM, and MMvd). Reproducibility was assessed through intraclass correlation coefficient (ICC), which was calculated for indices of hepatic disease for CM and MM as well as for key PK parameters for the CM. The ICC was single rater/measurement, two-way mixed effects model for absolute agreement among measurements [38, 39] with one-sided test for the lower acceptable limit (ICC>0.7) previously defined by a reduction in accuracy of <5% [37]. Correlation plots compare each method with MM and MMvd for key cholate test parameters with Deming regression fit to assess systematic differences [40] and coefficient of determination (R²) to assess relationship between the two methods.

Results and Discussion

Cholate second generation (2^{nd} gen) tests (DuO, TRIO, AI DuO, and AI TRIO) correlated well with previously validated liver function test parameters. All 2^{nd} gen methods resulted in similar or better reliability relative to the MM method in terms of CV and ICC. For all test parameters, the 2^{nd} gen tests correlated better with the MM based on V_{d} (MMvd) than with the original MM (except portal HER which was independent of V_{d}).

Portal HFR

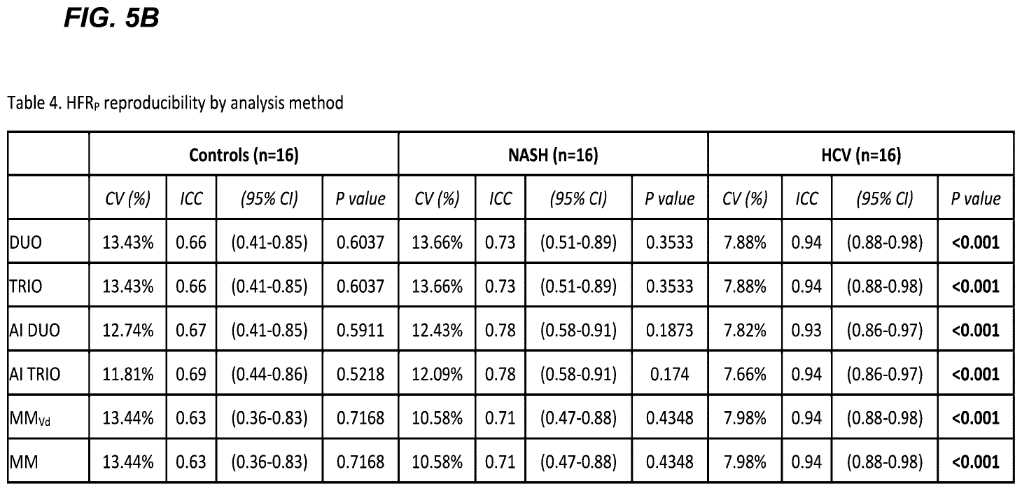

Six graphs illustrating correlation of portal hepatic filtration rate (HFR_{P}) among cholate 2^{nd} generation tests (DuO, TRIO, AI DuO, AI TRIO) and 1^{st} generation SHUNT test results analyzed by the MM (upper panels) and MM_{Vd} methods (lower panels) are shown in FIG. 5A. TRIO graphs are the same for those of DuO and are not shown.

In the case of portal HFR (FIG. 5A), information about the systemic clearance is not used in its calculation and, thus, DuO and TRIO methods are equivalent. Likewise, the volume of distribution has no influence on the fitting functions of the oral clearance curve, and as expected the MM and MM_{Vd} methods resulted in identical HFR_{P} values.

Table 3 shows HFR_{P} reproducibility by analysis method as measured by coefficient of variation (CV) and intraclass correlation coefficient (ICC) for all subjects (N=48).

TABLE 3
HFR_{P} reproducibility by analysis method

| | CV (%) | ICC | (95% CI) | P value |
|---|---|---|---|---|
| DuO | 11.65% | 0.84 | (0.76-0.90) | 0.0011 |
| TRIO | 11.65% | 0.84 | (0.76-0.90) | 0.0011 |
| AI DuO | 11.00% | 0.85 | (0.77-0.91) | <0.001 |
| AI TRIO | 10.52% | 0.86 | (0.79-0.91) | <0.001 |
| MM_{Vd} | 10.66% | 0.84 | (0.75-0.90) | 0.0017 |
| MM | 10.66% | 0.84 | (0.75-0.90) | 0.0017 |

Table 4 (FIG. 5B) shows HFR_{P} reproducibility by analysis method as measured by coefficient of variation (CV) and intraclass correlation coefficient (ICC) for patient groups (Controls, N=16; NASH, N=16; HCV, N=16).

All six methods resulted in similar reliability in terms of CV and ICC, with slightly better reproducibility measured in the AI DuO and AI TRIO methods across the entire study population (Table 3) and within patient groups (FIG. 5B, Table 4). These results suggest that the portal HFR can be measured with acceptable accuracy and reliability using the dual oral dosing method (DuO), using either the compartmental model or AI analysis.

Systemic HFR

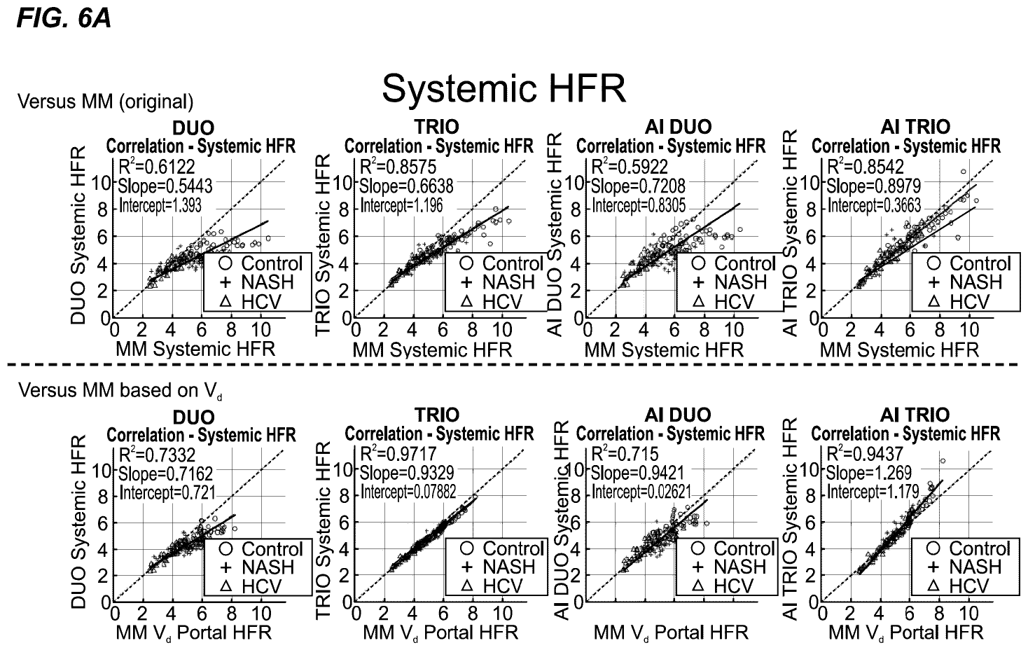

Eight graphs illustrating correlation of systemic HFR (HFRs) among cholate 2^{nd} generation tests (DuO. TRIO, AI DuO, AI TRIO) and 1^{st} generation SHUNT test results analyzed by the MM and MM_{Vd} methods are shown in FIG. 6A.

For systemic HFR (FIG. 6A), the addition of the 20-minute IV data point in TRIO led to a substantial improvement in the correlation with the MM (HFRs R² of 0.61 to 0.86 for DuO and TRIO, respectively).

Table 5 shows HFRs reproducibility by analysis method as measured by coefficient of variation (CV) and intraclass correlation coefficient (ICC) for all subjects (N=48).

TABLE 5
HFR_{S} reproducibility by analysis method

| | CV (%) | ICC | (95% CI) | P value |
|---|---|---|---|---|
| DuO | 2.96% | 0.97 | (0.95-0.98) | <0.001 |
| TRIO | 5.69% | 0.91 | (0.87-0.95) | <0.001 |
| AI DuO | 2.97% | 0.97 | (0.96-0.98) | <0.001 |
| AI TRIO | 7.86% | 0.89 | (0.82-0.93) | <0.001 |
| MM_{Vd} | 6.38% | 0.9 | (0.85-0.94) | <0.001 |
| MM | 11.00% | 0.82 | (0.73-0.89) | 0.008 |

Table 6 (FIG. 6B) shows HFRs reproducibility by analysis method as measured by coefficient of variation (CV) and intraclass correlation coefficient (ICC) for patient groups including NASH and HCV (Controls, N=16; NASH, N=16; HCV, N=16).

Additionally, all methods showed significantly better correlations with MM_{Vd} compared to MM. By far the best performing model was TRIO with an R² of 0.86 and 0.97 versus MM and MM_{Vd}, respectively. In terms of within-individual reproducibility, the 2^{nd} generation methods had significantly lower CV and better ICC relative to the MM (Table 5 and Table 6, FIG. 6B). Although the DuO and AI DuO had the lowest variability, these methods are less accurate than TRIO and AI TRIO methods which include the 20-minute IV data. The TRIO method had significantly lower variability compared to the MM method (CV of 5.7% and 11%; ICC of 0.91 and 0.82 for TRIO and MM, respectively). These results suggest that accurate and reliable systemic clearance curves can be generated using a single 20-minute timepoint and exponential fitting approach.

SHUNT

Since SHUNT is a ratio of AUC_{Oral} to AUC_{IV}, accurate and reliable estimates of both systemic and portal clearances are desired.

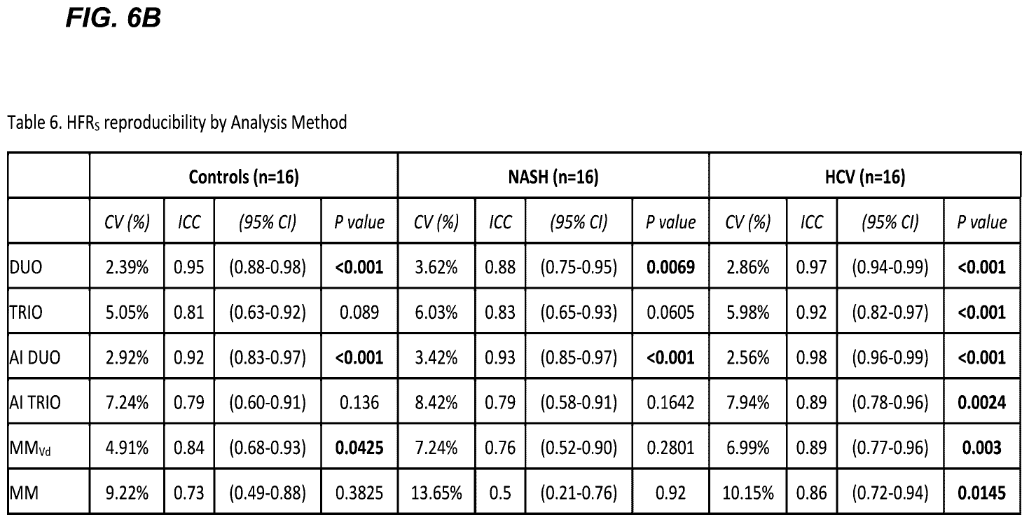

Eight graphs illustrating correlation of SHUNT % among cholate 2^{nd} generation tests (DuO, TRIO, AI DuO, AI TRIO) and 1^{st} generation cholate SHUNT test results analyzed by the MM and MMvd methods are shown in FIG. 7A.

Similar to systemic HFR, an improvement in correlation was found for SHUNT (FIG. 7A) when the IV 20-minute data point was added (SHUNT R² of 0.67 to 0.84 for DuO and TRIO, respectively). Additionally, all methods showed significantly better correlations with MM_{Vd} compared to MM.

By far the best performing model was TRIO with an R² of 0.84 and 0.96 versus MM and MM_{Vd}, respectively. In terms of within-individual reproducibility, the 2^{nd} gen methods had significantly lower CV and better ICC relative to the MM (Table 7 and Table 8).

Table 7 shows SHUNT reproducibility by analysis method as measured by coefficient of variation (CV) and intraclass correlation coefficient (ICC) for all subjects (N=48).

TABLE 7
SHUNT reproducibility by analysis method

| | CV (%) | ICC | (95% CI) | P value |
|---|---|---|---|---|
| DuO | 9.86% | 0.91 | (0.86-0.95) | <0.001 |
| TRIO | 12.24% | 0.86 | (0.79-0.91) | <0.001 |
| AI DuO | 8.90% | 0.91 | (0.85-0.94) | <0.001 |
| AI TRIO | 12.06% | 0.79 | (0.69-0.87) | 0.0328 |
| MM_{Vd} | 12.07% | 0.85 | (0.77-0.91) | <0.001 |
| MM | 15.32% | 0.73 | (0.60-0.83) | 0.3105 |

Table 8 (FIG. 7B) shows SHUNT reproducibility by analysis method as measured by coefficient of variation (CV) and intraclass correlation coefficient (ICC) for patient groups (Controls, N=16; NASH, N=16; HCV, N=16).

While all the 2^{nd} gen methods had reliable measurements for SHUNT, the MM failed to achieve an ICC of greater than 0.7 (p=0.3105). Again, the DuO and AI DuO had the lowest variability, but these methods are less accurate than TRIO and AI TRIO methods which include the 20-minute IV data. The TRIO method had lower variability compared to the MM method (CV of 12.1% and 15.3%; ICC of 0.79 and 0.73 for TRIO and MM, respectively). These results further support the finding that accurate and reliable measurements of both systemic and portal clearance curves can be generated using the TRIO approach.

DSI and HR

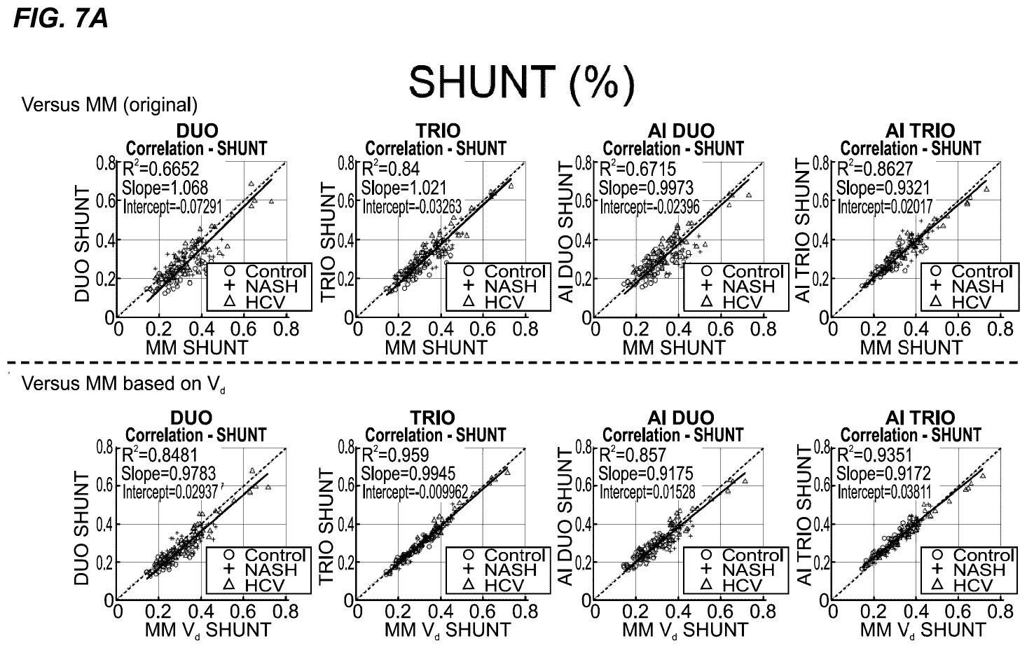

Eight graphs illustrating correlation of Disease Severity Index (DSI) among cholate 2^{nd} generation tests (DuO, TRIO, AI DuO, AI TRIO) and 1^{st} generation SHUNT test results analyzed by the MM and MMvd methods are shown in FIG. 8A.

Table 9 shows DSI reproducibility by analysis method as measured by coefficient of variation (CV) and intraclass correlation coefficient (ICC) for all subjects (N=48).

TABLE 9
DSI reproducibility by analysis method

| | CV (%) | ICC | (95% CI) | P value |
|---|---|---|---|---|
| DuO | 7.77% | 0.94 | (0.91-0.97) | <0.001 |
| TRIO | 8.28% | 0.95 | (0.92-0.97) | <0.001 |
| AI DuO | 8.52% | 0.94 | (0.91-0.97) | <0.001 |
| AI TRIO | 9.16% | 0.95 | (0.91-0.97) | <0.001 |
| MM_{Vd} | 8.60% | 0.95 | (0.92-0.97) | <0.001 |
| MM | 11.43% | 0.94 | (0.90-0.96) | <0.001 |

Table 10 (FIG. 8B) shows DSI reproducibility by analysis method as measured by coefficient of variation (CV) and intraclass correlation coefficient (ICC) for patient groups (Controls, N=16; NASH, N=16; HCV, N=16).

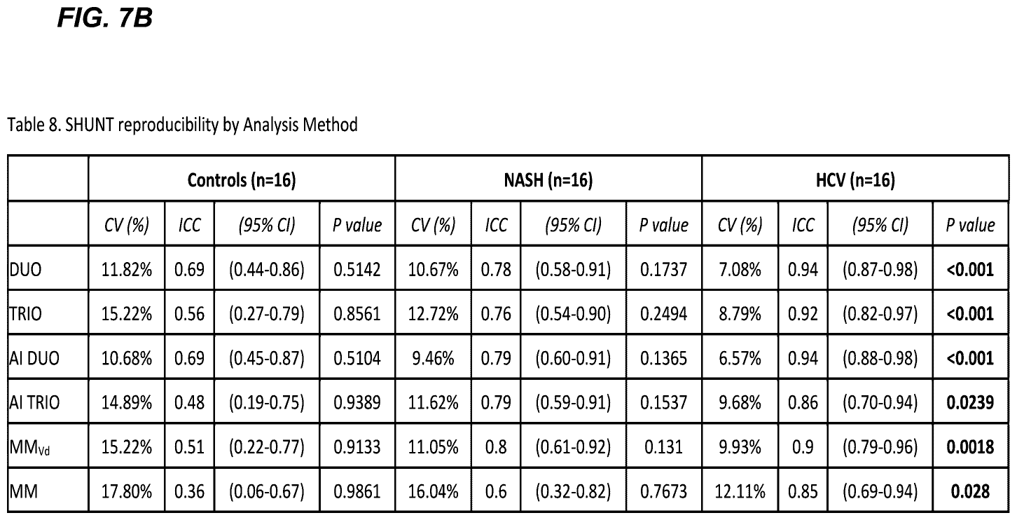

Eight graphs illustrating correlation of Hepatic Reserve (HR) among cholate 2^{nd} generation tests (DuO, TRIO, AI DuO, AI TRIO) and 1^{st} generation SHUNT test results analyzed by the MM and MMvd methods are shown in FIG. 9A.

Table 11 shows HR reproducibility by analysis method as measured by coefficient of variation (CV) and intraclass correlation coefficient (ICC) for all subjects (N=48).

TABLE 11
Hepatic Reserve reproducibility by analysis method

| | CV (%) | ICC | (95% CI) | P value |
|---|---|---|---|---|
| DuO | 2.08% | 0.95 | (0.93-0.97) | <0.001 |
| TRIO | 2.22% | 0.95 | (0.92-0.97) | <0.001 |
| AI DuO | 2.15% | 0.95 | (0.93-0.97) | <0.001 |
| AI TRIO | 2.16% | 0.95 | (0.93-0.97) | <0.001 |
| MM_{Vd} | 1.86% | 0.96 | (0.94-0.98) | <0.001 |
| MM | 1.88% | 0.97 | (0.94-0.98) | <0.001 |

Table 12 (FIG. 9B) shows HR reproducibility by analysis method as measured by coefficient of variation (CV) and intraclass correlation coefficient (ICC) for patient groups (Controls, N=16; NASH, N=16; HCV, N=16).

Although DSI (FIG. 8A) and HR (FIG. 9A) include both HFRs and HFR_{P} in their calculations, there was only a slight improvement in correlation between MM and 2^{nd} gen tests when the IV data was added (DSI R² of 0.94 to 0.97 for DuO and TRIO, respectively; HR R² of 0.97 to 0.98 for DuO and TRIO, respectively). Additionally, all methods showed slightly better correlations with MM_{Vd} compared to MM.

In terms of within-individual reproducibility of DSI, the 2^{nd} gen methods had significantly lower CV and better ICC relative to the MM (Table 9 and Table 10). This effect is especially pronounced in controls which have higher variability due to the heteroscedasticity of HFR_{S} (i.e., greater uncertainty in the LC-MS measurements at low concentrations). All methods had similar reproducibility characteristics for the measurement of HR, as the HR index truncates those with high HFR (i.e., values above the average of lean controls minus one standard deviation). These results suggest that both DuO and TRIO provide acceptable accuracy and reliability in measuring DSI and HR relative to the MM approach.

SHUNT V2.0 and Alternative DuO Analysis Methods

This section describes exemplary SHUNT V2.0 and alternative DuO analysis methods. SHUNT V2.0 is a simplification of the SHUNT test which measures 13C-CA (intravenous dose) and d4-CA (oral dose) concentrations at 20 and 60 minutes. SHUNT V2.0 uses exponential fits to systemic cholate clearance and compartmental analysis for portal cholate clearance based on assumptions of liver flow and physiology. DuO measures only d4-CA concentrations at 20 and 60 minutes to determine portal cholate clearance using the same compartmental model. A detailed description of the compartmental model which describes the compartmental model of the cholate SHUNT V1.0 test which allowed determination of anatomic shunting and hepatic extraction, as well as improved the within individual reproducibility of SHUNT test measurements is provided in McRae M P, Helmke S M, Burton J R, Jr., Everson G T. Compartmental model describing the physiological basis for the HepQuant SHUNT test. Transl Res. 2023; 252:53-63, which is incorporated by reference herein in its entirety. The following section highlights the key differences between the original model (for SHUNT V1.0) and two of the simplified models (SHUNT V2.0 and DuO).

Compartmental Model of Portal Clearance

The flow between systemic (S), portal (P), and liver (L) compartments was described by a system of first-order ordinary differential equations (Equations S1-S3) where q is the flow rate between compartments, V is the volume of the compartment, (is the concentration of cholate in the compartment, Cl_{H} is the hepatic clearance, and D_{PO,rate} is the rate of orally administered d4-cholate entering the portal compartment.

$$\frac{{dC}_{S}}{dt} = \frac{1}{{V}_{S}} ( - [ {q}_{SP} + {q}_{SL} ] {C}_{S} + {q}_{LS} \cdot {C}_{L} + {q}_{PS} \cdot {C}_{P} ) \qquad Eqn . S1$$

$$\frac{{dC}_{P}}{dt} = \frac{1}{{V}_{P}} ( {D}_{PO , rate} + {q}_{SP} \cdot {C}_{S} - {q}_{PL} \cdot {C}_{P} - {q}_{PS} \cdot {C}_{P} ) \qquad Eqn . S2$$

$$\frac{{dC}_{L}}{dt} = \frac{1}{{V}_{L}} ( - [ {Cl}_{H} \cdot {V}_{L} + {q}_{LS} ] {C}_{L} + {q}_{PL} \cdot {C}_{P} + {q}_{SL} \cdot {C}_{S} ) \qquad Eqn . S3$$

A key difference between the SHUNT V1.0 compartmental model and the simplified model is that the original model accounted for cholate binding. It is important to note that the HepQuant tests are measuring the flow-dependent and highly efficient uptake of cholate by the liver and not metabolism by the liver cells. In the case of HepQuant tests, the intravenously administered 13C-CA is pre-bound to albumin prior to administration to deter binding to cells/tissues, ensure its residency in the intravascular space, and facilitate efficient uptake by hepatocytes. It is assumed that the orally administered d4-CA is extensively bound to albumin upon intestinal absorption. While a fraction of the oral d4-CA dose may be exposed to binding by red blood cells and extravascular tissues, for simplification of the compartmental model the administered dose was assumed to enter and remain completely within the intravascular space and bound to albumin until hepatic extraction.

In health, the portal venous inflow to the liver (q_{PL}) equals the splanchnic arterial flow rate (q_{SP}). However, in the presence of collateral circulation (e.g., portosystemic shunts or esophageal and gastric varices), portal venous inflow is reduced by the shunt flow (q_{PS}) which bypasses the liver. To simplify the model for the purpose of fitting generalizable oral clearance curves to limited data points, the shunt flow rate was approximated by estimating a parameter representing the fraction of splanchnic circulation that is shunted (i.e., a constant between 0 and 1). The magnitude of the oral clearance curve was independently adjusted through the estimation of a scaling factor, F, which is related to the absolute bioavailability.

Hepatic extraction ratio (ER) is defined as the fraction of drug entering the liver which is irreversibly removed during a single pass through the liver [29, 30]. Free cholate is eliminated from the liver with total hepatic clearance defined as the hepatic inflow times the extraction ratio. Due to the high extraction ratio of cholate (ER>0.7) and relatively constant intrinsic hepatocyte clearance across the spectrum of liver disease, differences in cholate clearance in CID are assumed to be primarily attributed to altered flow to the liver. In the compartmental model, hepatic clearance, Cl_{H}, was calculated according to Equation S4.

Cl_{H}=Q_{L}·ER Eqn. S4

In the simplified compartmental model, rather than include ER as an estimated parameter, it was held constant at 0.75 and iteratively reduced by 5% if both the estimated parameters F and shunt flow fraction were greater than 50% to allow the model to fit data exhibiting reduced hepatocyte extraction.

Oral dose administration was modeled via a flexible transit model (Savic et al. Implementation of a transit compartment model for describing drug absorption in pharmacokinetic studies. J Pharmacokinet Pharmacodyn. 2007; 34:711-26; Wilkins et al. Population pharmacokinetic of rifampin in pulmonary tuberculosis patients, including a semimechanistic model to describe variable absorption. Antimicrob Agents Chemother. 2008; 52:2138-48). The flexible transit model has been demonstrated to closely describe absorption delay observed in oral drug administration. A transit model was adapted to describe the passage of D_{PO} through a series of n non-integer hypothetical transit compartments to simulate drug absorption delay and account for first-pass extraction. Equation S5 describes the rate of change of the amount of d4-CA (D_{PO,rate}) entering systemic circulation.

$${D}_{PO , rate} = {D}_{PO} \cdot F \cdot {k}_{TR} \cdot \frac{{( {k}_{TR} \cdot t )}^{n} \cdot {e}^{- {k}_{TR} \cdot t}}{\sqrt{2 π} \cdot {π}^{n + 0.5} \cdot {e}^{- n}} \qquad Eqn . S5$$

$${k}_{TR} = \frac{n + 1}{M T T} \qquad Eqn . S6$$

Here, t is time in minutes; k_{TR} is the transit rate constant between compartments (Equation S6); MTI is an estimated parameter representing the mean transit time of a d4-CA molecule through intestinal absorption and into systemic circulation (initial estimate of 30 minutes); F is an estimated parameter that scales the oral clearance curve and is related to the first-pass bioavailability (initial estimate of 0.20). Equations S5 and S6 define the shape of the oral clearance curve.

Noncompartmental Analysis of Systemic Clearance

The noncompartmental analysis for SHUNT V2.0 involves the exponential fits of systemic cholate clearance using only the 20-minute 13C-CA concentration timepoint. The exponential fits split the curve into three sections or phases: fast, moderate, and slow.

The first phase of clearance, between 0 and 20 minutes, represents a fast distribution phase. To estimate the initial 13C-CA concentration, the total blood volume (TBV) in liters per kg was first calculated by Equation S7 which accounts for the nonlinear relationship between blood volume and body mass index (BMI, kg/m²) across the entire range of body weights including obese (BM 1.30-40) and morbidly obese (BMI>40) subjects (Lemmens et al. Estimating blood volume in obese and morbidly obese patients. Obes Surg. 2006; 16:773-6).

$$T B V = \frac{0.07}{\sqrt{\frac{BMI}{22}}} \qquad Eqn . S7$$

In Equation S7, 22 is the BMI value corresponding to ideal body weight, and 0.07 is the indexed blood volume (in L/kg) for a subject with a BMI of 22. The initial 13C-CA concentration after dose administration was then estimated by dividing the IV dose by V_{d} times body weight (BW) (Equation S8).

$${C}_{0} = \frac{{D}_{IV}}{TBV \cdot BW} \qquad Eqn . S8$$

Finally, the rate of elimination in the fast phase is defined by k_{fast} (Equation S9) where C₂₀ is the concentration at 20 minutes, and Tw is the actual time recorded for the 20-minute sample.

$${k}_{fast} = - \frac{ln ( {C}_{20} ) - ln ( {C}_{0} )}{{T}_{20}} \qquad Eqn . S9$$

The second phase of clearance, between 20 and 60 minutes, represented a moderate elimination phase in which the elimination rate k_{mod} was defined by Equation S10.

$${k}_{mod} = - \frac{ln ( {C}_{60} ) - ln ( {C}_{20} )}{{T}_{60} - {T}_{20}} \qquad Eqn . S10$$

The final phase of clearance, at times greater than 60 minutes, represents a slow elimination phase in which the elimination rate k_{slow} was set to the mean value for SHUNT-V and control subjects, which was 0.0183 min⁻¹. The exponential equations defining the systemic concentration of 13C-CA through time were as follows:

$${Y}_{0} = {C}_{0} \cdot {e}^{- {k}_{fast} \cdot t} \qquad Eqn . S11$$

$${Y}_{1} = \frac{{C}_{20}}{{e}^{- {k}_{mod} \cdot {T}_{20}}} \cdot {e}^{- {k}_{mod} \cdot t} \qquad Eqn . S12$$

$${Y}_{2} = \frac{{C}_{60}}{{e}^{- {k}_{slow} \cdot {T}_{60}}} \cdot {e}^{- {k}_{slow} \cdot t} \qquad Eqn . S13$$

Here, 1 is time (0-20 min., 20-60 min., and 60-180 min. for Y₀, Y₁, and Y₂, respectively), C₀ is the initial concentration of 13C-CA, C₂₀ and C₆₀ are the measured 20- and 60-minute concentrations of 13C-CA, and T₂₀ and T₆₀ are the actual 20- and 60-minute sample collection times. The areas under each of the three exponential curve fits are calculated by trapezoidal numerical integration and summed to estimate the AUC_{IV}.

Estimation of IV Concentrations for DuO

For the oral-only simplified liver function test, DuO, the derived IV 20- and 60-minute concentrations were estimated by Equation S16 and Equation S17.

$${C}_{IV ( 20 )} = {β}_{0 , 20} + {β}_{BW , 20} \cdot ( \frac{BW - {Z}_{BW , mean}}{{Z}_{BW , SD}} ) + {β}_{BMI , 20} \cdot ( \frac{BMI - {Z}_{BMI , mean}}{{Z}_{BMI , SD}} ) + {β}_{PO ( 20 ) , 20} \cdot ( \frac{ln [ {C}_{PO ( 20 )} ] - {Z}_{PO ( 20 ) , mean}}{{Z}_{PO ( 20 ) , SD}} ) + {β}_{PO ( 60 ) , 20} \cdot ( \frac{ln [ {C}_{PO ( 60 )} ] - {Z}_{PO ( 60 ) , mean}}{{Z}_{PO ( 20 ) , SD}} ) + {β}_{T 20} \cdot ( \frac{{T}_{20} - {Z}_{T 20 , mean}}{{Z}_{T 20 , SD}} ) \qquad Eqn . S16$$

$${C}_{IV ( 60 )} = {β}_{0 , 60} + {β}_{BW , 60} \cdot ( \frac{BW - {Z}_{BW , mean}}{{Z}_{BW , SD}} ) + {β}_{PO ( 20 ) , 60} \cdot ( \frac{ln [ {C}_{PO ( 20 )} ] - {Z}_{PO ( 20 ) , mean}}{{Z}_{PO ( 20 ) , SD}} ) + {β}_{PO ( 60 ) , 60} \cdot ( \frac{ln [ {C}_{PO ( 60 )} ] - {Z}_{PO ( 60 ) , mean}}{{Z}_{PO ( 60 ) , SD}} ) \qquad Eqn . S17$$

The linear models were trained using data from the SHUNT-V Study (ClinicalTrials.gov. The SHUNT-V Study for Varices. clinicaltrials.gov/ct2/show/NCT03583996. Accessed Jul. 20, 2022.) (N=275) and healthy controls (N=50) and resulted in the standardization constants and regression coefficients listed in Table 14. The AUC_{IV} for DuO was then calculated by the same method as SHUNT V2.0, using instead estimated 20- and 60-minute IV concentrations rather than measured values.

TABLE 14
Linear regression model coefficients for estimating derived
20-minute and 60-minute concentrations of systemic 13C-CA
(IV) in the HepQuant cholate DuO liver function test.

| Coefficient | Description | Value |
|---|---|---|
| Standardization | | |
| constants | | |
| Z_{BW, mean} | Average body weight | 91.751005 |
| Z_{BW, SD} | SD of body weight | 23.621730 |
| Z_{BMI, mean} | Average body mass index | 32.043528 |
| Z_{BMI, SD} | SD of body mass index | 7.463782 |
| Z_{PO(20), mean} | Average of ln(13C-CA) at 20 | 0.198711 |
| | min | |
| Z_{PO(20), SD} | SD of ln(13C-CA) at 20 min | 0.943803 |
| Z_{PO(60), mean} | Average of ln(13C-CA) at 60 | −0.173990 |
| | min | |
| Z_{PO(60), SD} | SD of ln(13C-CA) at 60 min | 0.826406 |
| Z_{T20, mean} | Average of 20-min sample | 19.996920 |
| | times | |
| Z_{T20, SD} | SD of 20-min sample times | 0.876646 |
| 20-min. IV model | | |
| β_{0, 20} | Intercept | 0.553399 |
| β_{BW, 20} | Body weight | −0.090211 |
| β_{BMI, 20} | Body mass index | 0.039738 |
| β_{PO(20), 20} | Ln(d4-CA) at 20 min | 0.068113 |
| β_{PO(60), 20} | Ln(d4-CA) at 60 min | 0.340721 |
| β_{T20} | Time of 20-min sample | −0.029428 |
| 60-min. IV model | | |
| β_{0, 60} | Intercept | −0.600173 |
| β_{BW, 60} | Body weight | −0.015905 |
| β_{PO(20), 60} | Ln(d4-CA) at 20 min | 0.084136 |
| β_{PO(60), 60} | Ln(d4-CA) at 60 min | 0.473529 |

### Estimation of Portal HFR and DSI for the STAT Test

STAT is simply the d4-CA concentration at 60 minutes adjusted to 75 kg body weight by the calculation ([d4-CA]×(kg body weight/75 kg)). The value derived from the STAT test may be used by itself or in the estimation of portal HFR and DSI (FIG. 13).

To estimate portal HFR, Equation S14 was derived by fitting parameters to an exponential function to the portal HFR calculated from SHUNT V1.0.

HFR_{P,est}=2.1308+54.4412e^{-6.0001·STAT}+21.0936e^{-0.8578·STAT} Eqn. S14.

To estimate DSI, Equation S15 was derived by fitting parameters to a square root function to the DSI calculated from SHUNT V1.0.

DSIₑₛₜ=14.9051\sqrt{2.2930·STAT}−2.3052·STAT Eqn. S15.

### Use of DuO or TRIO Liver Function Tests for Measuring Treatment Effects

Changes in portal circulation may be detected by portal HFR, SHUNT %, first pass hepatic extraction of a distinguishable cholate, and DSI. A change in one or more of these parameters can detect a change in portal circulation. A cholate SHUNT test (SHUNT 1.0) with prior art minimal model data analysis was able to detect treatment effects in a clinical study. Lawitz, E., et al., BI 685509 improves hepatic function in subjects with Child-Pugh A cirrhosis and a liver stiffness measurement of >15 kPa: Results from the HepQuant SHUNT test. Hepatology, 2021(74): p. 1238A-1239A. The simpler inventive cholate DuO and TRIO tests may be used to detect the same treatment effects.

For example, the inventive DuO and/or TRIO methods provided herein may be used to monitor treatment effect in a subject receiving an investigational drug or known drug such as a nonselective beta adrenergic blocker, ACE inhibitor, guanylate cyclase activator, thyroid hormone receptor beta agonist (THR_{β} agonist), cyclophilin inhibitor, or other vasoactive drug used to modify hepatic and portal blood flow and portal systemic shunting, or a liver disease therapeutic to alter liver fat content, fibrosis, or liver cell function, or any other drug that may affect steatosis, of liver function.

### EXAMPLES

### Example 1. Simplified Cholate Tests: Reproducibility and Diagnostic Performance Data for Prediction of Large Esophageal Varices

### Example 1A. Simplified Cholate Tests: SHUNT V1.1 and SHUNT 2.0 for Prediction of Large Esophageal Varices

Background and Aims: The cholate SHUNT liver function test (minimal model, MM=SHUNT V1.0) uses stable isotopes of cholate administered both intravenously (13C-CA) and orally (d4-CA) labeled cholates to quantify liver function and physiology from blood sampled at 0, 5, 20, 45, 60, and 90 min for serum cholate. The MM cholate SHUNT Test has been used in over 26 clinical trials and studies, encompassing a broad range of etiologies and stages of liver disease, where it has compared favorably to other liver diagnostic tests. However, the MM cholate SHUNT Test is sensitive to variability in the timing of collection of the 5-minute blood sample and difficulty in maintaining intravenous access. The aim of this study was to enhance the MM cholate SHUNT Test and its performance by simplifying the sampling procedure and shortening the time of testing. Herein two new versions of the cholate test including MM_{Vd} (=SHUNT V1.1) and TRIO (V1.0) (=SHUNT V2.0) tests were evaluated.

Methods: In the MM cholate SHUNT test, the volume of distribution (V_{d}) is calculated from ln-linear regression of 5- and 20-minute 13C-CA concentrations versus time. The MM_{Vd} cholate test estimates V_{d} based on body weight and height (Lemmens et al. 2006), eliminating the requirement for the 5-minute blood sample. The TRIO (v 1.0) cholate test is based on published compartmental analysis (McRae et al. 2022) and further simplifies sampling requirements to 2 timepoints at 20 and 60 minutes. To compare reproducibility, coefficients of variation (CV) and intraclass correlation coefficients (ICC) with one-sided test for lower acceptable limit of 0.7 were analyzed in a study of 16 controls, 16 NASH patients, and 16 HCV patients, each with 3 replicate tests conducted on 3 separate days (Burton et al. 2021). The differences in areas under the receiver operator characteristic curve (AUROC) for predicting large esophageal varices (LEVs) in hepatitis C (HCV) subjects from the HALT-C study (N=217) (Everson et al. 2012) were assessed by the DeLong method. Test outputs include a Disease Severity Index (DSI) and portal-systemic shunting (SHUNT %).

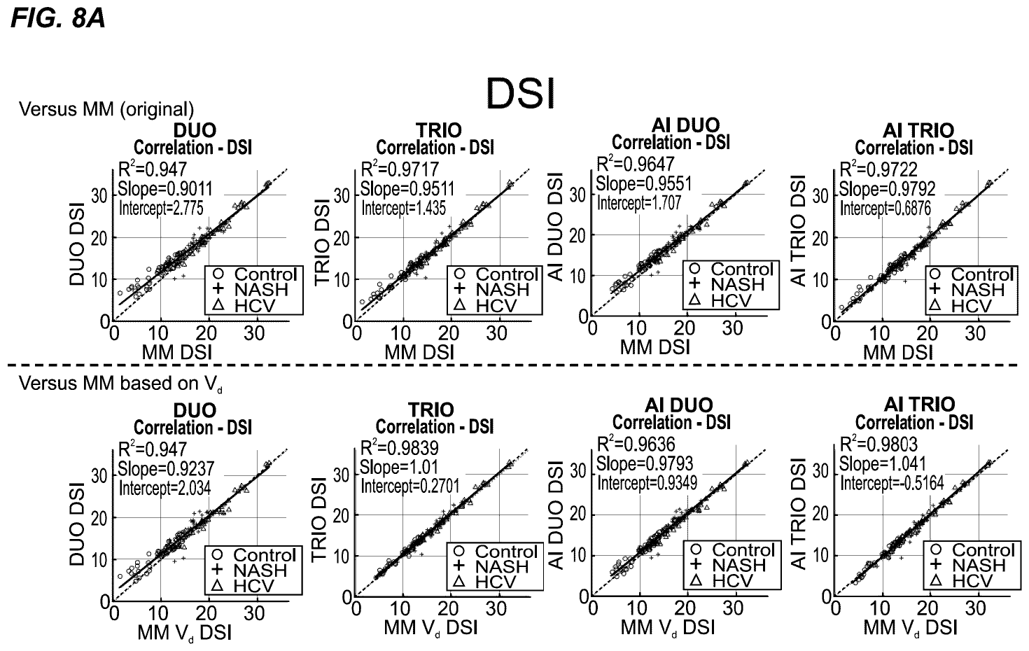

Results: For the measurement of DSI, MM_{Vd} and TRIO (v 1.0) cholate tests demonstrated similar ICCs but improved CVs relative to the MM cholate test method, as shown in FIG. 10A, Table 13A. For the measurement of SHUNT %, the MM_{Vd} and TRIO (v 1.0) cholate test versions demonstrated improved reproducibility based on both ICC and CV %. Diagnostic performance based on AUROCs for MM_{Vd} and TRIO (v 1.0) cholate test versions was equivalent to MM in most cases and improved in MM_{Vd} for SHUNT %.

Conclusion: The next generation of cholate tests MM_{Vd} and TRIO (v 1.0) simplify the test administration by eliminating the 5-minute sample (MM_{Vd}), reducing the total samples required to 2 (TRIO v 1.0), and shortening the time from 90 minutes to 60 minutes (TRIO v 1.0). These improvements should enhance operator performance, resource utilization, and patient acceptance of the cholate testing procedure, allowing for greater utilization of cholate tests for measuring liver function and physiology.

### Example 18. Simplified Cholate Tests: DuO Cholate Test for Prediction of Large Esophageal Varices

Background: Endoscopy (EGD) is indicated in patients with cirrhosis to check for large esophageal varices (LEVs) that need treatment. Prevalence of LEVs at EGD in Child-Pugh (CP) A cirrhosis is ˜10%. The cholate SHUNT test quantifies liver function and portal-systemic shunting. An oral-only version (DuO cholate test) was developed to simplify test administration and reduce variability.

Aims: Two aims of this study were to evaluate the diagnostic performance of the DuO cholate test in ruling out LEVs in CP A cirrhosis and to compare performance of HepQuant DuO and cholate SHUNT tests.

Methods: The subjects included 238 patients with CP A cirrhosis in the SHUNT-V Study including 52% MASLD/MASH, 25% HCV, and 16% alcoholic liver disease. The subjects included 64% obese, 87% overweight, and 54% diabetes.

SHUNT Test Administration: 13C-cholate was injected by IV and d4-cholate was administered orally to the subjects. Blood was sampled at 0, 5, 20, 45, 60, 90 min. for serum cholate. The simplified cholate test versions included cholate SHUNT V1.1 test and DuO cholate test. The SHUNT V1.1 cholate test included all 13C- and d4-CA concentrations except for the 5 min. data points and the 13C- and d4-CA concentrations were calculated based on cubic spline and exponential fits. Everson G T et al. Aliment. Pharmacol. Ther. 2007; 26:401-410. The DuO cholate test included only d4-CA concentrations at 20 and 60 min, calculated based on compartmental model. McRae M P et al. Transl. Res. 2023; 252:53-63.

Test parameters: Test parameters included disease severity index (DSI), portal-systemic shunt (SHUNT %), Hepatic Reserve, and Portal Hepatic Filtration Rate (HFR_{P}).

A DSI cutoff of <18.3 was prespecified based on >95% sensitivity for LEVs in the HALT-C Trial QLFT ancillary study.

Statistical analyses included: differences between subgroups analyzed by ANOVA (continuous data) and Chi-square (categorical data); DSI from lean and overweight controls plotted alongside subjects with no, small, or large esophageal varices; diagnostic performance for ruling out LEVs (AUROC, sensitivity, specificity, PPV, NPV); AUROCs for DuO; and SHUNT V1.1 for ruling out LEVs compared by DeLong method; and univariate logistic regression of DSI for presence of LEVs.

Results: Laboratory values, clinical scores, and cholate test parameters calculated from DuO in SHUNT-V CP A subjects for all subjects (n=238), subjects with no varices (n=135), small varices (n=76), and large varices (n=27) are shown in Table 13B.

The simplified DuO cholate test parameters including DSI, SHUNT %, Hepatic Reserve, and HFRp were each sensitive in detecting presence and size of varices as shown in Table 13B.

TABLE 13B
Laboratory Values, Clinical Scores, and Cholate
Test Parameters from DuO in SHUNT-V CP A Subjects

| | All Subjects | No Varices | Small Varices | Large Varices | |
|---|---|---|---|---|---|
| Laboratory Values | (N = 238) | (N = 135) | (N = 76) | (N = 27) | p-value |
| Albumin, g dL⁻¹ | 4.23 ± 0.43 | 4.31 ± 0.41 | 4.18 ± 0.45 | 4.00 ± 0.42 | 0.0014 |
| Alk. Phos., U L⁻¹ | 100.53 ± 51.86 | 94.61 ± 37.64 | 102.87 ± 69.34 | 123.11 ± 50.22 | 0.0294 |
| ALT, U L⁻¹ | 38.42 ± 34.24 | 39.90 ± 41.00 | 37.32 ± 24.28 | 34.22 ± 18.15 | 0.6945 |
| AST, U L⁻¹ | 42.11 ± 24.98 | 41.03 ± 25.69 | 42.47 ± 25.28 | 46.44 ± 20.56 | 0.5855 |
| Bilirubin, mg dL⁻¹ | 0.75 ± 0.44 | 0.70 ± 0.41 | 0.74 ± 0.43 | 1.00 ± 0.52 | 0.0042 |
| Creatinine, mg dL⁻¹ | 0.90 ± 0.29 | 0.89 ± 0.25 | 0.93 ± 0.38 | 0.89 ± 0.19 | 0.6377 |
| Platelets, ×10³ μL⁻¹ | 152 ± 68 | 169 ± 68 | 142 ± 61 | 97 ± 53 | <0.0001 |
| Prothrombin time, | 1.09 ± 0.15 | 1.08 ± 0.13 | 1.11 ± 0.18 | 1.13 ± 0.10 | 0.1076 |
| INR | | | | | |
| APRI | 0.87 ± 0.66 | 0.72 ± 0.53 | 0.87 ± 0.62 | 1.56 ± 0.92 | <0.0001 |
| FIB-4 | 3.63 ± 2.67 | 2.98 ± 2.06 | 3.70 ± 2.23 | 6.61 ± 4.10 | <0.0001 |
| Clinical Scores | | | | | |
| Child-Pugh score | 5.44 ± 0.35 | 5.11 ± 0.32 | 5.12 ± 0.33 | 5.33 ± 0.48 | 0.0076 |
| MELD score | 7.84 ± 2.22 | 7.58 ± 1.93 | 8.15 ± 2.69 | 8.27 ± 1.93 | 0.1198 |
| HepQuant DuO | | | | | |
| DSI | 22.56 ± 7.53 | 20.49 ± 6.64 | 23.64 ± 7.71 | 29.88 ± 6.07 | <0.0001 |
| SHUNT % (%) | 38.57 ± 15.61 | 34.23 ± 13.29 | 40.97 ± 16.30 | 53.52 ± 14.02 | <0.0001 |
| Hepatic Reserve (%) | 71.33 ± 19.23 | 76.84 ± 17.05 | 68.27 ± 19.18 | 52.40 ± 15.94 | <0.0001 |
| HFR_{P} (mL/min/kg) | 11.02 ± 6.86 | 12.54 ± 6.29 | 10.29 ± 7.70 | 5.52 ± 2.94 | <0.0001 |

DSI from lean and overweight controls was plotted alongside subjects with no, small, or large esophageal varices. A monotonic, stepwise increase in DSI with increasing risk for LEVs was exhibited, as shown in FIG. 10B.

DSI values measured by DuO cholate test in Child-Pugh A cirrhosis subjects and control subjects vs. probability of LEVs based on DSI are shown in FIG. 10C. DSI cutoff of 18.3 is shown as a vertical line. DSI from DuO demonstrated significant association with finding LEVs at endoscopy (p<0.001).

Diagnostic performance (95% CI) of DuO (DSI<18.3) in SHUNT-V CP A Subjects (n=238) is shown in Table 13C.

Applying a DSI<18.3 cutoff from DuO to SHUNT-V subjects would have missed only 1 LEV case compared to DSI from SHUNT V1.1, and prevented 84 EGDs.

TABLE 13C
Diagnostic Performance (95% CI) of DuO (DSI <
18.3) in SHUNT-V CP A Subjects (n = 238)

| | | |
|---|---|---|
| Sensitivity | 0.96 | (0.81-1.00) |
| Specificity | 0.39 | (0.33-0.46) |
| Positive Predictive Value | 0.17 | (0.15-0.19) |
| Negative Predictive Value | 0.99 | (0.92-1.00) |
| Positive Likelihood Ratio | 1.59 | (1.39-1.81) |
| Negative Likelihood Ratio | 0.09 | (0.01-0.65) |
| Missed LEVs, n (%)* | 1 | (3.7%) |
| EGDs Prevented, n (%) | 84 | (35.3%) |

*1 missed case detected by SHUNT V1.1

A pairwise comparison of AUROC for DSI from DuO or SHUNT V1.1 in detecting LEVs in SHUNT-V CPA subjects (n 238) is shown in Table 13D illustrating DuO was equivalent to SHUNT V1.1 in detecting the presence of LEVs.

TABLE 13D
Pairwise comparison of AUROC for DSI in
detecting LEVs in SHUNT-V CP A (N = 238)

| | AUROC (95% CI) | p value |
|---|---|---|
| DuO | 0.809 (0.737-0.882) | 0.3169 |
| SHUNT V1.1 | 0.817 (0.749-0.885) | — |

Conclusions:

The simplified cholate DuO test parameters of liver function and physiology correlate with presence and size of esophageal varices. Knowing the likelihood that a given DSI is associated with a particular risk of LEVs is highly relevant to clinical decision making. DuO and SHUNT V1.1 were statistically equivalent in the detection of LEVs. DuO missed one LEV case that was detected by SHUNT V1.1. Application of DSI<18.3 would have avoided EGDs in 35% for DuO versus 31% for SHUNT V1.1. DuO is easier to administer and less invasive, thus, having the potential to be more widely accepted by care providers administering the test and by patients receiving the test.

### Example 2. Reproducibility of Simplified Liver Function Tests

In this example, the reproducibility of simplified liver function tests is described relative to that of SHUNT V1.0. This study was a retrospective analysis of data from two reproducibility studies of the HepQuant SHUNT test (236 tests completed in 94 subjects), the HepQuant Reproducibility Study (REPRO) and the Primary Sclerosing Cholangitis (PSC) Study.

This example explores the reproducibility of the DuO cholate test, an oral-only cholate challenge test, and other simplified versions of the prototypical SHUNT V1.0. The simplified tests, SHUNT V1.1, SHUNT V2.0, and DuO cholate tests, reduce blood draws to four (SHUNT V1.1) or two samples (SHUNT V2.0 and DuO) made possible by a compartmental model (McRae et al., Transl Res. 2023; 252:53-63), and in the case of DuO, eliminates the IV injection. The within-individual reproducibility of the simplified tests was measured.

Subjects

The REPRO study comprised three groups of subjects: healthy persons without liver disease, patients with NASH, and patients with HCV. The NASH group was diagnosed based on risk factors (obesity, diabetes, and metabolic syndrome), negative tests for other liver diseases, and fibrosis stage determined by either liver biopsy or transient elastography. The HCV group was diagnosed based on a positive HCV history through nucleic acid testing and METAVIR fibrosis stage determined by liver biopsy. Three separate HepQuant SHUNT tests were conducted on three different days within a 30-day period.

Data obtained from the PSC Study (Everson G T, Helmke S M. Defining disease severity and measuring progression in primary sclerosing cholangitis (PSC): a comparison of the disease severity index (DSI) from the HepQuant SHUNT test with serum alkaline phosphatase (alk phos). Hepatology. 2018; 68:1087A) was analyzed retrospectively. Reproducibility was assessed by conducting two baseline SHUNT tests in 46 subjects representing the clinical spectrum of PSC.

The REPRO and PSC studies were conducted according to The Code of Ethics of the World Medical Association (Declaration of Helsinki). Informed consent was obtained from all subjects. The REPRO study was approved by the Colorado Multi-Institutional Review Board and registered at ClinicalTrials.gov, NCT01579162. The PSC study was approved by the Institutional Review Board of the University of Colorado Denver, and cholate isotopes were studied under IND 65121 (13C-CA) and IND 65123 (d4-CA).

Reproducibility Analysis

Reproducibility of test parameters was assessed through intraclass correlation coefficient (ICC), coefficient of variation (CV), and minimum detectable difference (MDD). The ICC method was single rater/measurement, two-way mixed effects model for absolute agreement among measurements (Koo et al. Guideline of Selecting and Reporting Intraclass Correlation Coefficients for Reliability Research. J Chiropr Med. 2016; 15:155-63; McGraw et al. Forming inferences about some intraclass correlation coefficients. Psychol Methods. 1996; 1:30-46) with one-sided test for the lower acceptable limit (ICC>0.7) previously defined by a reduction in accuracy of <5%. (Burton et al. The within-individual reproducibility of the disease severity index from the HepQuant SHUNT test of liver function and physiology. Transl Res. 2021; 233:5-15.)

Acceptable reproducibility was determined by ICC above the lower acceptable limit of 0.7 (p<0.05). The MDD, interpreted as the smallest detectable difference in value of a test parameter before and after experimental manipulation (e.g., treatment) which is larger than would be expected by chance (Matheson G J. We need to talk about reliability: making better use of test-retest studies for study design and interpretation. Peer J. 2019; 7:e6918), was calculated for each test parameter. Weir J P. Quantifying test-retest reliability using the intraclass correlation coefficient and the SEM. J Strength Cond Res. 2005; 19:231-40. Baumgartner et al. Statistical evaluation of test-retest studies in PET brain imaging. EJNMMI Research. 2018; 8:13.

The reproducibility was assessed for each study independently (three paired tests per subject for REPRO and two paired tests per subject for PSC) and for both studies combined. For the combined analysis, three pairings of two tests per subject were derived for the REPRO study data according to the study visits (V), i.e., V1-V2, V1-V3, and V2-V3.

### Results

### Subject Characteristics

REPRO Study. The REPRO Study comprised 48 subjects who were categorized into three groups: controls (N=16), NASH (N=16), and HCV (N=16). Subject characteristics (Table 15) and standard laboratory test results (Table 16) from the REPRO Study (Burton et al., Transl Res. 2021; 233:5-15) were reproduced here. The control group had an average age of 32.9±12.0 years, a balanced male-to-female ratio of 8:8, and a normal BMI of 23.0±2.2. The NASH group had an average age of 49.8±11.6 years, had a male-to-female ratio of 7:9 (M:F), and a high BMI of 32.5±5.9. The HCV group had an average age of 55.6±6.7 years, a male-to-female ratio of 13:3, and an intermediate BMI of 28.2±4.1. Most subjects (40 out of 48) were non-Hispanic white. Compared to controls, both the NASH and HCV groups had significantly higher levels of alanine aminotransferase (ALT), aspartate aminotransferase (AST), gamma-glutamyl transferase (GGT), and glucose, as well as lower platelet counts. The NASH and HCV groups were classified based on fibrosis stage as either F0-F2 (early) or F3-F4 (advanced), with 8 subjects in each stage. All F4 subjects had well compensated liver disease without history of clinical complications.

TABLE 15
Standard laboratory tests for subjects in the HepQuant Reproducibility
(REPRO) Study and the Primary Sclerosing Cholangitis (PSC)
Study. Data are displayed as mean ± SD.

| | REPRO Study^{†} | | | |
|---|---|---|---|---|
| Laboratory tests | Controls | NASH | HCV | PSC Study |
| Bilirubin (mg/dL) | 0.7 ± 0.3 | 0.8 ± 0.4 | 1.1 ± 0.4** | 1.9 ± 2.1 |
| Albumin (g/dL) | 4.0 ± 0.3 | 4.0 ± 0.3 | 3.9 ± 0.4 | 3.6 ± 0.5 |
| INR | 1.0 ± 0.1 | 1.0 ± 0.1 | 1.1 ± 0.1 | 1.15 ± 0.56 |
| Creatinine (mg/dL) | 1.0 ± 0.1 | 0.8 ± 0.3** | 0.9 ± 0.1 | 0.90 ± 0.20 |
| AST (IU/mL) | 22.3 ± 4.8 | 34.0 ± 17.7* | 75.7 ± 64.0** | 59 ± 41 |
| ALT (IU/mL) | 20.7 ± 7.3 | 43.7 ± 33.4* | 100.9 ± 91.4** | 60 ± 45 |
| GGT (IU/mL) | 18.0 ± 6.9 | 44.4 ± 28.4** | 75.8 ± 83.7** | 229 ± 258 |
| Alkaline phosphatase | 52 ± 17 | 65 ± 24 | 69 ± 30* | 233 ± 263 |
| (IU/mL) | | | | |
| Platelet count (nL⁻¹) | 243 ± 47 | 193 ± 72* | 152 ± 68*** | 184 ± 90 |
| Hemoglobin (g/dL) | 14.3 ± 1.4 | 14.6 ± 1.7 | 15.1 ± 1.9 | 14.3 ± 1.8 |
| WBC (nL⁻¹) | 5.3 ± 1.3 | 6.0 ± 1.8 | 5.9 ± 2.7 | 5.2 ± 1.9 |
| Glucose (g/dL) | 84.1 ± 7.0 | 106.3 ± 33.1* | 101.2 ± 16.0*** | NA |
| TSH | 1.6 ± 0.5 | 2.1 ± 1.2 | 2.4 ± 2.2 | NA |
| Cholesterol, total | 162 ± 30 | 167 ± 45 | 143 ± 23 | NA |
| Triglyceride | 79 ± 41 | 168 ± 86** | 105 ± 51 | NA |
| Ferritin | 65 ± 62 | 126 ± 117 | 332 ± 279*** | NA |

Abbreviations: ALT, alanine aminotransferase; AST, aspartate aminotransferase; GGT, gamma-glutamyl transferase; HCV, cases with chronic hepatitis C; INR, international normalized ratio for prothrombin time; N, number; NASH, cases with nonalcoholic steatohepatitis; TSH, thyroid stimulating hormone; WBC, white blood cell count.
*P < 0.05.
**P < 0.01.
***P < 0.001 by 2-sided t-tests, compared to controls.
^{†}From the REPRO Study (Burton, James R. et al.)

PSC Study. Subject characteristics (Table 16) and standard laboratory test results (Table 15) from the PSC Study were recorded. Of the 47 participants in the study, 43 were non-Hispanic white, 3 were African American, and 1 was Hispanic. The male to female ratio was 36:11, with an average age of 48.8±12.9 years, body weight of 81.3±14.9 kg, and BMI of 26.2±3.9 kg/m². Inflammatory bowel disease (IBD) was present in 70% (33) of the participants, with 76% (25) having ulcerative colitis. Among the participants, 36% (17) had a history of varices, ascites, or encephalopathy, with 15 having varices, 2 having variceal hemorrhage, 5 having diuretic-responsive ascites, and 8 having treatment-responsive encephalopathy. Radiologic reports indicated that 36% (17) had splenomegaly, and 28%(13) had a platelet count less than 140,000 μL⁻¹. Additionally, 19% (9) had a history of jaundice, 38% (18) had a history of bacterial cholangitis, and 72% (28 out of 39) of those with endoscopic retrograde cholangiopancreatography (ERCP) reports had undergone stricture dilation, stenting, or both. Treatment with ursodeoxycholic acid was administered to 66% (31) of the participants, while 40% (19) were taking IBD medications.

TABLE 16
Selected characteristics of study subjects from the HepQuant
Reproducibility (REPRO) Study and Primary Sclerosing Cholangitis
(PSC) Study. Data are displayed as N or mean ± SD.

| | REPRO Study^{‡} | | | |
|---|---|---|---|---|
| Characteristics | Controls | NASH | HCV | PSC Study |
| No. of subjects | 16 | 16 | 16 | 47 |
| Age (y) | 32.9 ± 12.0 | 49.8 ± 11.6 | 55.6 ± 6.7 | 48.8 ± 12.9 |
| Gender (M:F) | 8:8 | 7:9 | 13:3 | 36:11 |
| BMI (kg/m²) | 23.0 ± 2.2 | 32.5 ± 5.9 | 28.2 ± 4.1 | 26.2 ± 3.9 |
| Race/ethnicity (NHW:H:B:U) | 12:2:1:1 | 14:2:0:0 | 14:1:1:0 | 43:1:3:0 |
| Fibrosis stage, N subjects | NA | 0:4:4:5:3 | 3:2:3:4:4 | NA |
| (F0:F1:F2:F3:F4)* | | | | |
| Fibrosis score* | NA | 2.4 ± 1.1 | 2.3 ± 1.5 | NA |
| MELD score^{†} | NA | 7.0 ± 1.1 | 7.9 ± 2.0 | 9.5 ± 4.2 |
| CTP score^{†} | NA | 5.0 ± 0.0 | 5.4 ± 0.7 | 6.0 ± 1.5 |

Abbreviations: B, Black race; BMI, body mass index; CTP, Child-Turcotte-Pugh; H, Hispanic; HCV, cases with chronic hepatitis C; M:F, male to female ratio; MELD, model for end-stage liver disease; N, number; NA, not applicable—healthy controls did not undergo liver biopsy and none had clinical disease and fibrosis stage was not reported in the PSC study; NASH, cases with nonalcoholic steatohepatitis; NHW, non-Hispanic white; U, undocumented.
*METAVIR in HCV cases; BRUNT/KLEINER in NASH cases; 2 NASH cases had F3 fibrosis by transient elastography.
^{†}MELD and CTP scores were calculated for the F3/F4 subgroup only and in the PSC Study.
^{‡}From the REPRO Study (Burton, James R. et al.)

### Reproducibility

REPRO Study. In the REPRO Study (Table 17) (N=48 subjects), all test parameters were reproducible (ICC>0.7, p<0.05) for test versions SHUNT V1.1, SHUNT V2.0, and DuO. SHUNT % and RCA-20 did not meet reproducibility criteria for SHUNT V1.0. STAT, Portal HFR, and DSI were reproducible for test version STAT.

TABLE 17
Reproducibility of test parameters calculated by various test versions for
all subjects in the HepQuant Reproducibility Study (N = 48). CV, coefficient
of variation; MDD, minimum detectable difference; ICC, intra-class correlation
coefficient with 95% confidence interval and p-value testing ICC > 0.7

| Parameter | Test Version | CV | MDD | ICC (95% CI) | p-value |
|---|---|---|---|---|---|
| DSI | SHUNT V1.0 | 11.07% | 1.47 | 0.94 (0.90-0.96) | <0.001 |
| | SHUNT V1.1 | 9.62% | 1.25 | 0.94 (0.91-0.97) | <0.001 |
| | SHUNT V2.0 | 10.36% | 1.43 | 0.94 (0.90-0.96) | <0.001 |
| | DuO | 10.64% | 1.60 | 0.93 (0.89-0.96) | <0.001 |
| SHUNT % (%) | SHUNT V1.0 | 14.93% | 10.21 | 0.74 (0.63-0.84) | 0.2120 |
| | SHUNT V1.1 | 11.92% | 5.83 | 0.85 (0.78-0.91) | <0.001 |
| | SHUNT V2.0 | 12.64% | 6.22 | 0.84 (0.76-0.90) | 0.0011 |
| | DuO | 9.18% | 3.35 | 0.90 (0.84-0.94) | <0.001 |
| Portal HFR (mL min⁻¹ | SHUNT V1.0 | 10.40% | 5.18 | 0.84 (0.76-0.90) | <0.001 |
| kg⁻¹) | SHUNT V1.1 | 10.48% | 5.36 | 0.84 (0.76-0.90) | 0.0013 |
| | SHUNT V2.0 | 11.59% | 6.05 | 0.82 (0.73-0.89) | 0.0056 |
| | DuO | 11.59% | 6.05 | 0.82 (0.73-0.89) | 0.0056 |
| Systemic HFR (mL | SHUNT V1.0 | 11.15% | 1.21 | 0.82 (0.73-0.89) | 0.0068 |
| min⁻¹ kg⁻¹) | SHUNT V1.1 | 6.48% | 0.47 | 0.90 (0.84-0.94) | <0.001 |
| | SHUNT V2.0 | 6.36% | 0.46 | 0.90 (0.85-0.94) | <0.001 |
| | DuO | 4.47% | 0.25 | 0.94 (0.90-0.96) | <0.001 |
| Hepatic Reserve (%) | SHUNT V1.0 | 2.16% | 1.71 | 0.96 (0.94-0.98) | <0.001 |
| | SHUNT V1.1 | 2.30% | 1.92 | 0.96 (0.94-0.98) | <0.001 |
| | SHUNT V2.0 | 2.54% | 2.54 | 0.95 (0.92-0.97) | <0.001 |
| | DuO | 2.60% | 2.65 | 0.95 (0.91-0.97) | <0.001 |
| RCA-20 (%) | SHUNT V1.0 | 20.06% | 12.15 | 0.38 (0.20-0.56) | 0.9999 |
| | SHUNT V1.1 | 10.11% | 1.95 | 0.89 (0.83-0.93) | <0.001 |
| | SHUNT V2.0 | 10.11% | 1.95 | 0.89 (0.83-0.93) | <0.001 |
| | DuO | 6.45% | 1.21 | 0.92 (0.88-0.95) | <0.001 |
| Risk ACE (events per | SHUNT V1.0 | 18.57% | 0.24 | 0.98 (0.97-0.99) | <0.001 |
| person-year) | SHUNT V1.1 | 16.32% | 0.19 | 0.99 (0.98-0.99) | <0.001 |
| | SHUNT V2.0 | 18.08% | 0.24 | 0.98 (0.97-0.99) | <0.001 |
| | DuO | 18.81% | 0.26 | 0.98 (0.97-0.99) | <0.001 |
| STAT (μM) | STAT | 18.20% | 0.19 | 0.90 (0.84-0.94) | <0.001 |
| STAT-estimated DSI | STAT | 8.34% | 2.1 | 0.88 (0.81-0.92) | <0.001 |
| STAT-estimated | STAT | 13.53% | 5.18 | 0.82 (0.73-0.89) | 0.0066 |
| Portal HFR (mL min⁻¹ | | | | | |
| kg⁻¹) | | | | | |

Reproducibility of test parameters was assessed across fibrosis stages (N=16 subjects for each fibrosis group and each subject category). Test parameters were calculated from AUCs of the serum concentrations of cholate isotopes. AUCs increased with increasing stage of fibrosis and were lowest in healthy persons and in patients with low stages of fibrosis. Low AUCs were subject to greater variance. As a result, ICC's were higher for every test parameter and test version with increasing stage of fibrosis (data not shown) and in subjects with disease compared to controls (data not shown). DSI, Hepatic Reserve, and Risk-ACE met reproducibility criteria across F0-F2 fibrosis for SHUNT V1.0 and SHUNT V1.1 and across F3-F4 for all test versions (p<0.001). Point estimates of ICCs generally exceeded the reproducibility cutoff of 0.70 across all test parameters and test versions although not by statistically significant (p<.) amounts in every case.

PSC Study. In the PSC Study (Table 18) (N=46 subjects), all test parameters, except Systemic HFR and RCA-20, met criteria for reproducibility (ICC>0.7, p<0.05) across all test versions (p<0.001).

TABLE 18
Reproducibility of test parameters calculated by various test versions for all
subjects in the Primary Sclerosing Cholangitis Study (N = 46). CV, coefficient
of variation; MDD, minimum detectable difference; ICC, intra-class correlation
coefficient with 95% confidence interval and p-value testing ICC > 0.7.

| Parameter | Test Version | CV | MDD | ICC (95% CI) | p-value |
|---|---|---|---|---|---|
| DSI | SHUNT V1.0 | 8.28% | 2.03 | 0.93 (0.88-0.96) | <0.001 |
| | SHUNT V1.1 | 8.13% | 1.89 | 0.94 (0.89-0.96) | <0.001 |
| | SHUNT V2.0 | 9.61% | 2.37 | 0.92 (0.86-0.95) | <0.001 |
| | DuO | 9.24% | 2.19 | 0.92 (0.87-0.96) | <0.001 |
| SHUNT % (%) | SHUNT V1.0 | 10.69% | 5.80 | 0.92 (0.86-0.96) | <0.001 |
| | SHUNT V1.1 | 9.79% | 4.64 | 0.93 (0.88-0.96) | <0.001 |
| | SHUNT V2.0 | 10.76% | 5.80 | 0.91 (0.85-0.95) | <0.001 |
| | DuO | 9.00% | 4.60 | 0.92 (0.86-0.96) | <0.001 |
| Portal HFR (mL min⁻¹ | SHUNT V1.0 | 11.30% | 2.65 | 0.90 (0.83-0.94) | <0.001 |
| kg⁻¹) | SHUNT V1.1 | 11.28% | 2.67 | 0.90 (0.82-0.94) | <0.001 |
| | SHUNT V2.0 | 13.23% | 3.76 | 0.86 (0.77-0.92) | 0.0017 |
| | DuO | 13.23% | 3.76 | 0.86 (0.77-0.92) | 0.0017 |
| Systemic HFR (mL min⁻¹ | SHUNT V1.0 | 10.38% | 1.40 | 0.79 (0.64-0.88) | 0.0984 |
| kg⁻¹) | SHUNT V1.1 | 8.49% | 0.87 | 0.81 (0.69-0.89) | 0.0341 |
| | SHUNT V2.0 | 8.79% | 0.96 | 0.79 (0.65-0.88) | 0.0820 |
| | DuO | 5.86% | 0.44 | 0.88 (0.79-0.93) | <0.001 |
| Hepatic Reserve (%) | SHUNT V1.0 | 4.70% | 4.32 | 0.94 (0.90-0.97) | <0.001 |
| | SHUNT V1.1 | 4.71% | 4.27 | 0.94 (0.90-0.97) | <0.001 |
| | SHUNT V2.0 | 5.08% | 5.08 | 0.93 (0.88-0.96) | <0.001 |
| | DuO | 5.04% | 4.98 | 0.93 (0.88-0.96) | <0.001 |
| RCA-20 (%) | SHUNT V1.0 | 19.75% | 19.34 | 0.54 (0.30-0.72) | 0.9567 |
| | SHUNT V1.1 | 14.29% | 7.95 | 0.72 (0.54-0.83) | 0.4117 |
| | SHUNT V2.0 | 14.29% | 7.95 | 0.72 (0.54-0.83) | 0.4117 |
| | DuO | 7.83% | 2.25 | 0.92 (0.86-0.96) | <0.001 |
| Risk ACE (events per | SHUNT V1.0 | 20.65% | 5.41 | 0.89 (0.81-0.94) | <0.001 |
| person-year) | SHUNT V1.1 | 20.53% | 4.69 | 0.91 (0.84-0.95) | <0.001 |
| | SHUNT V2.0 | 23.46% | 4.39 | 0.89 (0.81-0.94) | <0.001 |
| | DuO | 22.69% | 4.15 | 0.90 (0.82-0.94) | <0.001 |
| STAT (μM) | STAT | 20.87% | 0.33 | 0.92 (0.86-0.95) | <0.001 |
| STAT-estimated DSI | STAT | 9.62% | 3.1 | 0.89 (0.80-0.94) | <0.001 |
| STAT-estimated Portal | STAT | 16.91% | 8.23 | 0.70 (0.52-0.82) | 0.4989 |
| HFR (mL min⁻¹ kg⁻¹) | | | | | |

Combined. Overall, HepQuant test parameters were highly reproducible across test versions (ICC values ˜0.94 for DSI, ˜0.89 for SHUNT %, ˜0.95 for Hepatic Reserve). In test parameters which emphasize systemic clearance (Systemic HFR, SHUNT %, RCA-20), the CV and MDD decreased in the following order of highest to lowest variability: SHUNT V1.0, SHUNT V2.0, SHUNT V1.1, DuO. In both studies combined, DuO had substantially lower MDD for SHUNT % (3.56) than SHUNT V1.0 (7.80)(Table 19). In test parameters which emphasize the portal clearance (Portal HFR, DSI, Hepatic Reserve, Risk ACE), the CV and MDD either slightly increased or stayed the same in SHUNT V2.0 and DuO. For Hepatic Reserve, which had the lowest CV among all test parameters at approximately ˜2-3%, SHUNT V1.0 and SHUNT V1.1 were similar in terms of MDD (2.47 and 2.58, respectively), while SHUNT V2.0 and DuO were slightly higher (3.24 and 3.28, respectively). For DSI, SHUNT V1.1 had the lowest MDD (1.40) followed by SHUNT V1.0, SHUNT V2.0, and DuO (1.60, 1.67, and 1.73, respectively).

TABLE 19
Reproducibility of HepQuant liver function test parameters calculated by various
SHUNT test versions for all subjects in both the HepQuant Reproducibility Study
and Primary Sclerosing Cholangitis Study combined (N = 94 subjects). CV,
coefficient of variation; MDD, minimum detectable difference; ICC, intra-class
correlation coefficient with 95% confidence interval and p-value testing ICC > 0.7.

| Parameter | Test Version | CV | MDD | ICC (95% CI) | p-value |
|---|---|---|---|---|---|
| DSI | SHUNT V1.0 | 9.66% | 1.6 | 0.94 (0.92-0.95) | <0.001 |
| | SHUNT V1.1 | 8.65% | 1.39 | 0.94 (0.93-0.96) | <0.001 |
| | SHUNT V2.0 | 9.36% | 1.66 | 0.93 (0.91-0.95) | <0.001 |
| | DuO | 9.45% | 1.72 | 0.93 (0.91-0.95) | <0.001 |
| SHUNT % (%) | SHUNT V1.0 | 12.65% | 7.8 | 0.85 (0.81-0.89) | <0.001 |
| | SHUNT V1.1 | 10.56% | 5.14 | 0.90 (0.86-0.92) | <0.001 |
| | SHUNT V2.0 | 11.13% | 5.76 | 0.88 (0.84-0.91) | <0.001 |
| | DuO | 8.43% | 3.6 | 0.91 (0.89-0.93) | <0.001 |
| Portal HFR (mL min⁻¹ | SHUNT V1.0 | 9.93% | 4.57 | 0.86 (0.81-0.89) | <0.001 |
| kg⁻¹) | SHUNT V1.1 | 9.98% | 4.72 | 0.85 (0.81-0.89) | <0.001 |
| | SHUNT V2.0. | 11.18% | 5.48 | 0.83 (0.78-0.87) | <0.001 |
| | DuO | 11.18% | 5.48 | 0.83 (0.78-0.87) | <0.001 |
| Systemic HFR (mL min⁻¹ | SHUNT V1.0 | 10.09% | 1.26 | 0.81 (0.75-0.85) | <0.001 |
| kg⁻¹) | SHUNT V1.1 | 6.42% | 0.56 | 0.88 (0.84-0.91) | <0.001 |
| | SHUNT V2.0 | 6.42% | 0.58 | 0.88 (0.84-0.91) | <0.001 |
| | DuO | 4.43% | 0.29 | 0.92 (0.90-0.94) | <0.001 |
| Hepatic Reserve (%) | SHUNT V1.0 | 2.62% | 2.46 | 0.96 (0.94-0.97) | <0.001 |
| | SHUNT V1.1 | 2.72% | 2.57 | 0.95 (0.94-0.97) | <0.001 |
| | SHUNT V2.0 | 2.95% | 3.23 | 0.94 (0.92-0.96) | <0.001 |
| | DuO | 3.00% | 3.28 | 0.94 (0.92-0.96) | <0.001 |
| RCA-20 (%) | SHUNT V1.0 | 18.11% | 12.8 | 0.56 (0.46-0.65) | 0.9991 |
| | SHUNT V1.1 | 10.38% | 3.76 | 0.82 (0.77-0.86) | <0.001 |
| | SHUNT V2.0 | 10.38% | 3.76 | 0.82 (0.77-0.86) | <0.001 |
| | DuO | 6.25% | 1.45 | 0.93 (0.90-0.95) | <0.001 |
| Risk ACE (events per | SHUNT V1.0 | 17.48% | 2.39 | 0.91 (0.88-0.93) | <0.001 |
| person-year) | SHUNT V1.1 | 16.07% | 2.07 | 0.93 (0.90-0.94) | <0.001 |
| | SHUNT V2.0 | 17.74% | 1.94 | 0.92 (0.89-0.94) | <0.001 |
| | DuO | 18.01% | 1.84 | 0.92 (0.90-0.94) | <0.001 |
| STAT (μM) | STAT | 17.23% | 0.22 | 0.92 (0.89-0.94) | <0.001 |
| STAT-estimated DSI | STAT | 7.97% | 2.33 | 0.89 (0.85-0.91) | <0.001 |
| STAT-estimated Portal | STAT | 13.39% | 5.83 | 0.80 (0.75-0.85) | <0.001 |
| HFR (mL min⁻¹ kg⁻¹) | | | | | |

The simplified methods were found to be highly reproducible across test parameters with intraclass correlation coefficients >0.93 for key test parameters (DSI and Hepatic Reserve). SHUNT V2.0 and DuO improved reproducibility in measuring portal-systemic shunting (SHUNT %).

### Example 3. Equivalency of Simplified Liver Function Tests to Cholate SHUNT Tests

Equivalency of a Dual Sample Oral Cholate Challenge Test (DuO) and other simplified versions of cholate SHUNT test to Cholate SHUNT Test was investigated.

Current noninvasive liver tests primarily serve as surrogates for fibrosis and lack the ability to directly measure liver function. Cholate SHUNT liver function tests directly address this issue by leveraging the hepatic uptake of stable cholate isotopes to measure liver function and physiology. Because the HepQuant SHUNT test (V1.0/1.1) is complicated to administer, simplified test versions, SHUNT V2.0 (oral and IV dosing, but only 2 blood samples at 20 and 60 minutes) and DuO (oral dosing only, 2 blood samples at 20 and 60 minutes) have been developed as described herein. Excellent reproducibility and reliability of a single test within a given individual is also described herein.

In the present example, equivalency of these simplified tests to the original cholate SHUNT liver function test was investigated. Data from three studies comprising 930 cholate SHUNT tests in 372 subjects were analyzed retrospectively by each method.

The cholate SHUNT liver function test is a unique testing platform that is suitable for addressing the spectrum of liver disease etiologies and severities through the evaluation of both hepatocyte function and portal circulation. Everson et al., Portal-systemic shunting in patients with fibrosis or cirrhosis due to chronic hepatitis C: the minimal model for measuring cholate clearances and shunt. Aliment Pharmacol Ther. 2007; 26:401-10. Everson et al., The spectrum of hepatic functional impairment in compensated chronic hepatitis C: results from the Hepatitis C Anti-viral Long-term Treatment against Cirrhosis Trial. Aliment Pharmacol Ther. 2008; 27:798-809. Despite the strong promise of the cholate SHUNT liver function test, its implementation involves administering both oral and intravenous (IV) cholate doses and collecting five peripheral venous blood samples over a 90-minute period. This process introduces potential sources of error, such as challenges with IV access, the risk of extravasation, timing errors during sample collection, and variability in the administration skills of the test.

A compartmental model for the dual cholate clearance assay was previously developed, enhancing its reproducibility and interpretation of hepatic extraction and portal-systemic shunt. McRae et al., Compartmental model describing the physiological basis for the HepQuant SHUNT test. Transl Res. 2023; 252:53-63.

Using the compartmental modelling approach, simplified versions of the cholate SHUNT test were developed, SHUNT V2.0 and DuO, which require fewer blood draws, reduce the testing time, simplify the test administration, and enhance the reproducibility of test parameters. The cholate SHUNT V1.1 liver function test, which eliminates the need for the 5-minute sample, was found to be more reproducible than SHUNT V1.0 and, thus, was designated as the reference method for the present equivalency evaluation. The present example investigates whether the simplified DuO and SHUNT V2.0 tests are equivalent to the reference (SHUNT V1.1) method.

Methods

This study was a retrospective analysis of data collected from three different studies of the HepQuant SHUNT test: the HepQuant Reproducibility Study (REPRO)(Burton et al. The within-individual reproducibility of the disease severity index from the HepQuant SHUNT test of liver function and physiology. Transl Res. 2021; 233:5-15), the Primary Sclerosing Cholangitis (PSC) Study (Everson and Helmke. Defining disease severity and measuring progression in primary sclerosing cholangitis (PSC): a comparison of the disease severity index (DSI) from the HepQuant SHUNT test with serum alkaline phosphatase (alk phos). Hepatology. 2018; 68:1087A), and the Hepatitis C Antiviral Long-Term Treatment Against Cirrhosis Trial (HALT-C) Quantitative Liver Function Test (QLFT) ancillary study (Everson et al. Quantitative liver function tests improve the prediction of clinical outcomes in chronic hepatitis C: Results from the hepatitis C antiviral long-term treatment against cirrhosis trial. Hepatology. 2012; 55:1019-29).

Subjects

The REPRO study included 48 subjects from three groups (16 controls, 16 NASH, and 16 HCV) who underwent three separate SHUNT tests on three different days within a 30-day period, representing a total of 144 SHUNT tests performed in the study. The healthy controls had no prior history of liver disease and normal standard blood tests. NASH was diagnosed based on risk factors, absence of evidence for other liver diseases, and fibrosis stage by liver biopsy or transient elastography. HCV was diagnosed based on confirmed HCV history by nucleic acid testing and METAVIR fibrosis stage by liver biopsy.

The PSC study included 47 patients with PSC who were recruited from hepatology clinics representing the clinical spectrum of PSC. All 47 subjects had a baseline SHUNT test, 46 had a second baseline test to assess reproducibility, and 40 had a follow up test after one year, representing a total of 133 SHUNT tests performed in the study.

Details of the HALT-C QLFT study have been previously published (Everson et al. Hepatology. 2012; 55:1019-29). Cholate SHUNT tests were performed in 277 subjects at baseline, 212 at Year 2, and 164 at Year 4. A total of 653 SHUNT tests were performed in the HALT-C study.

Cholate Liver Function Test Versions

The Cholate Liver Function Test versions are summarized below. Cholate SHUNT V1.0 liver function test involves the simultaneous administration of 13C-CA by IV and d4-CA orally and six timed peripheral venous blood samples over 90 minutes. Cholate concentrations in serum are measured by LC/MS, and a noncompartmental analysis fits IV and oral clearance curves to measurement data calculate the areas under the IV and oral curves (AUC_{IV}, AUC_{Oral}). (Everson et al. Portal-systemic shunting in patients with fibrosis or cirrhosis due to chronic hepatitis C: the minimal model for measuring cholate clearances and shunt. Aliment Pharmacol Ther. 2007; 26:401-10).

Cholate SHUNT V1.1 liver function test eliminates the 5-minute sample from the calculations. Instead, the 13C-CA is estimated from a total blood volume calculation (Lemmens et al., Estimating blood volume in obese and morbidly obese patients. Obes Surg. 2006; 16:773-6), and the 5-minute d4-CA is approximated by 15% of the 20-minute concentration. This modification was shown to significantly reduce the variability in measuring test parameters that depend on the systemic clearance. For this reason, SHUNT V1.1 is designated as the reference method in this example.

Cholate SHUNT V2.0 liver function test further simplifies by shortening the testing window and requiring only two blood draws at 20 and 60 minutes. Cholate SHUNT V2.0 uses a compartmental model (McRae et al., Transl Res. 2023; 252:53-63) to measure the portal cholate clearance and noncompartmental exponential fits to measure systemic cholate clearance. DuO cholate liver function test is an oral-only test involving only one oral dose at 0 minutes and two blood samples collected at 20 and 60 minutes. The IV clearance is derived rather than measured, and the derived IV concentrations are then used in the same noncompartmental analysis as SHUNT V2.0.

Cholate STAT liver function test is the simplest test method which involves one oral cholate dose and one blood sample at 60 minutes. The value of the STAT test may be reported as either the STAT score (e.g., the d4-CA concentration adjusted to 75 kg body weight), the portal HFR estimated by STAT, or DSI estimated by STAT.

### Test Parameters

The various cholate liver function tests return a series of test parameters which have been previously associated with liver function and disease/health status. This study assessed the following cholate test parameters for their equivalency to the reference method:

Portal hepatic filtration rate (HFR_{P}) is the portal clearance adjusted for body weight (units mL min⁻¹ kg⁻¹) and was calculated by Equation 7B:

$$H F {R}_{P} = \frac{{D}_{PO}}{{AUC}_{Oral} \cdot BW} , \qquad Eqn . 7 B$$

where D_{PO} is the oral cholate dose.

Systemic hepatic filtration rate (HFR_{S}) is the measured systemic clearance adjusted for bod weight units mL min⁻¹ kg⁻¹) and was calculated by Equation 12:

$$H F {R}_{S} = \frac{{D}_{IV}}{{AUC}_{IV} \cdot BW} , \qquad Eqn . 12$$

where D_{IV} is the intravenous cholate dose.

Disease Severity Index (DSI) is a score between 0 and 50 which indicates overall liver function comprising both portal and systemic HFR. DSI is associated with fibrosis stage and clinical stages of cirrhosis [24] and was calculated by Equation 14:

$$D S I = A \cdot \sqrt{{( ln [ \frac{H F {R}_{P , max}}{H F {R}_{P}} ] )}^{2} + {( ln [ \frac{H F {R}_{S , max}}{H F {R}_{S}} ] )}^{2}} , \qquad Eqn . 14$$

where A is a scaling factor and HFR_{P,max} and HFR_{S,max} are the upper limits of clearance for healthy controls.

SHUNT %, or shunt fraction, is the estimated absolute bioavailability of the oral d4-CA dose in systemic circulation estimated by Equation 9B. SHUNT % is a direct measurement of the first pass hepatic extraction of cholate which is influenced by portal blood flow and portal-systemic shunt.

$$S H U N T \% = \frac{A U {C}_{Oral} \cdot {D}_{IV}}{A U {C}_{IV} \cdot {D}_{PO}} . \qquad Eqn . 9 B$$

Hepatic Reserve (HR) represents an individual's overall hepatic health relative to lean healthy controls, with values between 100 (healthy) and 0 (severely impaired). HR was calculated by Equation 15:

$$H R = 100 - A \cdot \sqrt{{( ln [ \frac{H F {R}_{P , lean}}{H F {R}_{P}} ] )}^{2} + {( ln [ \frac{H F {R}_{S , lean}}{H F {R}_{S}} ] )}^{2}} , \qquad Eqn . 15$$

where A is a scaling factor and HFR_{P,lean} and HFR_{S,lean} are mean HFRs minus one standard deviation from a population of lean controls.

### Statistical Analysis

The cholate liver function tests may potentially be affected by variance in the administration of test compounds and time of sampling, day-to-day fluctuations in gastrointestinal or hepatic physiology, and variations in sample preparation and laboratory analysis. The upper and lower equivalence bounds were specified based on the smallest effect size of interest defined by the 95% confidence interval for within-individual variability from the REPRO Study as shown in Table 20.

TABLE 20
Within-individual standard deviations (SD) of
differences and equivalence bounds for all test
parameters for SHUNT V1.0 and SHUNT V1.1.

| | | Within- | |
|---|---|---|---|
| | Reference | individual | Equivalence |
| Test Parameter | Method | SD | Bounds (±) |
| DSI | SHUNT V1.0 | 2.12 | 4.15 |
| | SHUNT V1.1 | 1.92 | 3.75 |
| SHUNT % (%) | SHUNT V1.0 | 0.07 | 0.14 |
| | SHUNT V1.1 | 0.06 | 0.11 |
| Hepatic Reserve (%) | SHUNT V1.0 | 3.27 | 6.42 |
| | SHUNT V1.1 | 3.46 | 6.79 |
| Portal HFR (mL min⁻¹ | SHUNT V1.0 | 4.73 | 9.26 |
| kg⁻¹) | SHUNT V1.1 | 4.82 | 9.44 |
| Systemic HFR (mL min⁻¹ | SHUNT V1.0 | 1.03 | 2.01 |
| kg⁻¹) | SHUNT V1.1 | 0.53 | 1.05 |

To evaluate agreement, correlation plots compared analysis methods with the reference method (SHUNT V1.1) for determination of test parameters using Deming regression fit to assess systematic differences. Cornbleet et al., Incorrect least-squares regression coefficients in method-comparison analysis. Clin Chem. 1979; 25:432-8.

Bland-Altman plots compared test parameters calculated by each analysis method and the reference method for bias and 95% confidence interval (Cl). Bland J M, Altman D G. Measuring agreement in method comparison studies. Stat Methods Med Res. 1999; 8:135-60.

The primary performance measure was the proportion of tests/subjects in which the difference between analysis methods was less than any clinically meaningful difference. The analysis was completed for each study individually and for all datasets combined.

The FDA has also provided guidance on preferred methods to demonstrate equivalence of tests—a two one-sided t-tests (TOST) (Lakens D. Equivalence Tests: A Practical Primer for t Tests, Correlations, and Meta-Analyses. Social Psychological and Personality Science. 2017; 8:355-62; Rogers et al., Using significance tests to evaluate equivalence between two experimental groups. Psychol Bull. 1993; 113:553-65), and bioequivalence procedures (U.S. Department of Health and Human Services, Food and Drug Administration, Center for Drug Evaluation and Research. Statistical Approaches to Establishing Bioequivalence—Guidance for Industry. Revision 1 ed2022).

In the TOST procedure the upper and lower equivalence bound was specified based on the smallest effect size of interest (Table 20). Two composite null hypotheses were tested. If both one-sided tests are statistically rejected, the observed effect falls within the equivalence bounds and is statistically smaller than any effect deemed worthwhile and considered practically equivalent. To evaluate bioequivalence, the mean ratio and 90% confidence intervals of the next generation tests and V1.0 was calculated for each study separately and across all studies. The mean ratios and 90% CIs were compared to bioequivalence limits of 80 and 120% for parameters in their original scale (i.e., not log-transformed).

Subject Characteristics

REPRO Study. The REPRO Study involved 48 participants divided into three groups: controls (N=16), NASH (N=16), and HCV (N=16). The control group had a mean age of 33±12 years, an equal distribution of males and females (8:8), and a normal BMI of 23±2. The NASH group exhibited a mean age of 50±12 years, male-to-female ratio of 7:9, and elevated BMI of 33±6. The HCV group had mean age of 56±7 years, male-to-female ratio of 13:3, and moderate BMI of 28±4. The majority of participants (83%) were non-Hispanic white. Alanine aminotransferase (ALT), aspartate aminotransferase (AST), gamma-glutamyl transferase (GGT), and glucose were elevated in NASH and HCV relative to controls. Platelets were significantly lower in NASH and HCV. Both NASH and HCV groups had 8 F0-F2 (early) fibrosis and 8±3-F4 (advanced) fibrosis.

PSC Study. Out of the 47 subjects in the PSC study, 92% were non-Hispanic white, 6% were African American, and 2% identified as Hispanic. The majority of subjects were male (36:11 M:F) with mean age of 49±13 years, body weight of 81±15 kg, and BMI of 26±4 kg/m². Seventy percent of subjects had inflammatory bowel disease, 53% had ulcerative colitis, 36% had history of varices, ascites, or encephalopathy, 36% had splenomegaly, 28% had low platelet count (<140,000 μL⁻¹), 19% had history of jaundice, and 38% had history of bacterial cholangitis. Seventy-two percent of those with ERCP reports (28 out of 39) had undergone stricture dilation, stenting, or both. Sixty-six percent of subjects were treated with UDCA, and 40% were taking medication for inflammatory bowel disease.

HALT-C QLFT Study. Out of the 285 chronic HCV patients enrolled in the HALT-C Trial QLFT ancillary study, 60% had Ishak fibrosis stages 2-4 (fibrosis) and 40% had stage S or 6 (cirrhosis). No subjects had experienced clinical decompensation. The majority of subjects were male (76%) with a mean age of 49.9±7.2 years and mean BMI of 29.4±4.8. Ninety-two percent had HCV genotype I and mean (iSD) HCV-RNA of 6.4 (±0.56) log₁₀ of IU/mL. The mean (iSD) platelet count was 169,000 (±69,000) μL⁻¹.

Equivalency to SHUNT V1.1

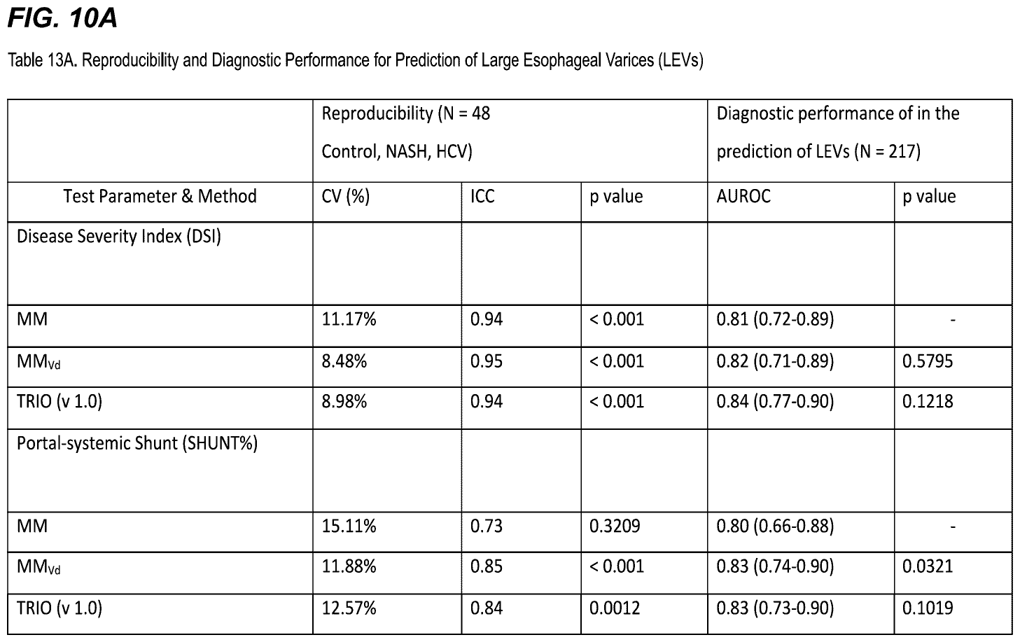

The equivalence of SHUNT V2.0, DuO, and STAT relative to SHUNT V1.1 are summarized in Table 21 which show the percentage of tests that fall within the equivalence bands for each study individually and across all studies. Equivalency in DSI between simplified test versions and SHUNT V1.1 was further assessed by TOST and bioequivalence methods (FIG. 14A-D).

TABLE 21
Equivalence of simplified cholate liver function test methods to SHUNT V1.1 in terms
of the number of tests (% of tests) falling within the equivalence bounds.

| | | REPRO | | PSC | | HALT-C | | Combined | |
|---|---|---|---|---|---|---|---|---|---|
| Parameter | Test Version | (N = 144) | | (N = 133) | | (N = 653) | | (N = 930) | |
| DSI | SHUNT V2.0 | 144 | (100) * | 132 | (99) * | 647 | (99) * | 923 | (99) * |
| | DuO | 144 | (100) * | 131 | (99) * | 623 | (95.41) * | 898 | (97) * |
| | STAT | 112 | (78) | 117 | (88) | 565 | (87) | 794 | (85) |
| SHUNT % (%) | SHUNT V2.0 | 144 | (100) * | 132 | (99) * | 639 | (98) * | 915 | (98) * |
| | DuO | 133 | (92) | 103 | (77) | 554 | (85) | 790 | (85) |
| | STAT | NA | | NA | | NA | | NA | |
| Hepatic Reserve | SHUNT V2.0 | 144 | (100) * | 131 | (99) * | 625 | (96) * | 900 | (97) * |
| (%) | DuO | 144 | (100) * | 129 | (97) * | 606 | (93) | 879 | (95) * |
| | STAT | NA | | NA | | NA | | NA | |
| Portal HFR | SHUNT V2.0 | 144 | (100) * | 132 | (99) * | 645 | (99) * | 921 | (99) * |
| (mL min⁻¹ kg⁻¹) | DuO | 144 | (100) * | 132 | (99) * | 645 | (99) * | 921 | (99) * |
| | STAT | 137 | (95) * | 131 | (99) * | 639 | (98) * | 907 | (98) * |
| Systemic HFR (mL | SHUNT V2.0 | 144 | (100) * | 133 | (100) * | 653 | (100) * | 930 | (100) * |
| min⁻¹ kg⁻¹) | DuO | 111 | (77) | 101 | (76) | 555 | (85) | 767 | (82) |

| | | | | |
|---|---|---|---|---|
| STAT | NA | NA | NA | NA |

* Above acceptable limit (≥95%)

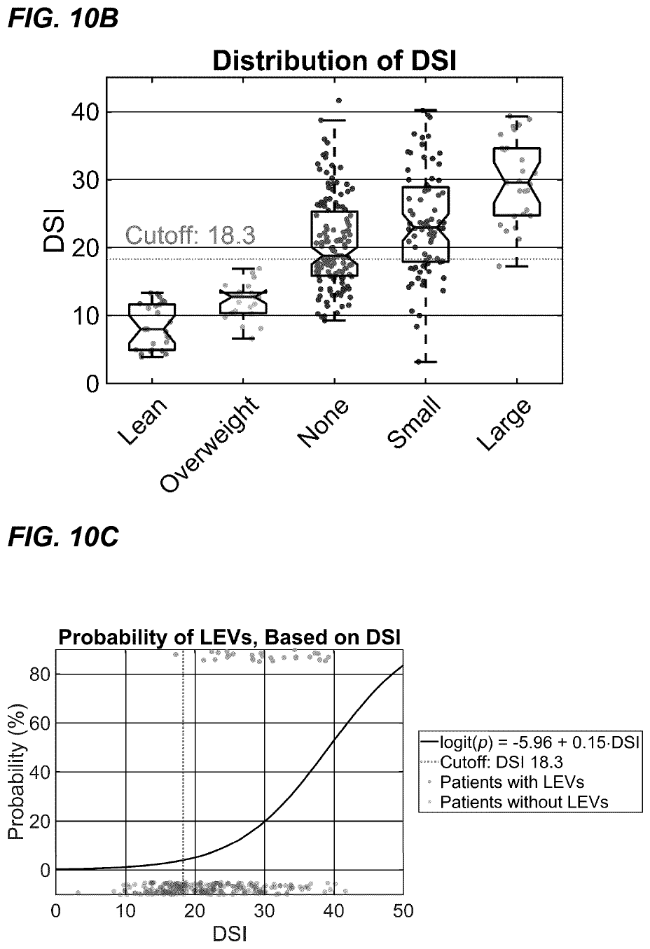

SHUNT V2.0. SHUNT V2.0 was equivalent to the reference method for all test parameters with the percentage of tests within the equivalence bounds ranging from 97% to 100%. The DSI measured by SHUNT V2.0 had excellent agreement with the reference method in terms of DSI (FIG. 15A-15B), with low bias (0.21 DSI units) and all but seven tests falling within the equivalence bounds and Deming regression line approaching identity (R²=0.98, slope=0.99, intercept=−0.05). The DSI from SHUNT V2.0 was also statistically equivalent to, not different from, and bioequivalent to the DSI measured from SHUNT V1.1, across all studies individually and combined (FIG. 14B). The SHUNT % measured by SHUNT V2.0 also had excellent agreement with the reference method with mean bias of 1.3% and R² of 0.96 (Data not shown).

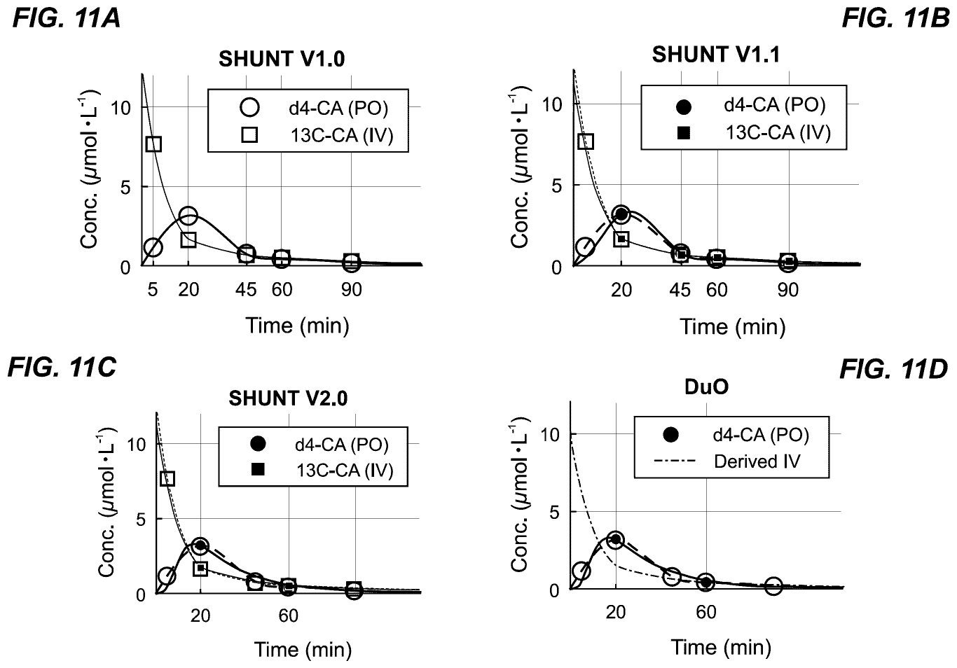

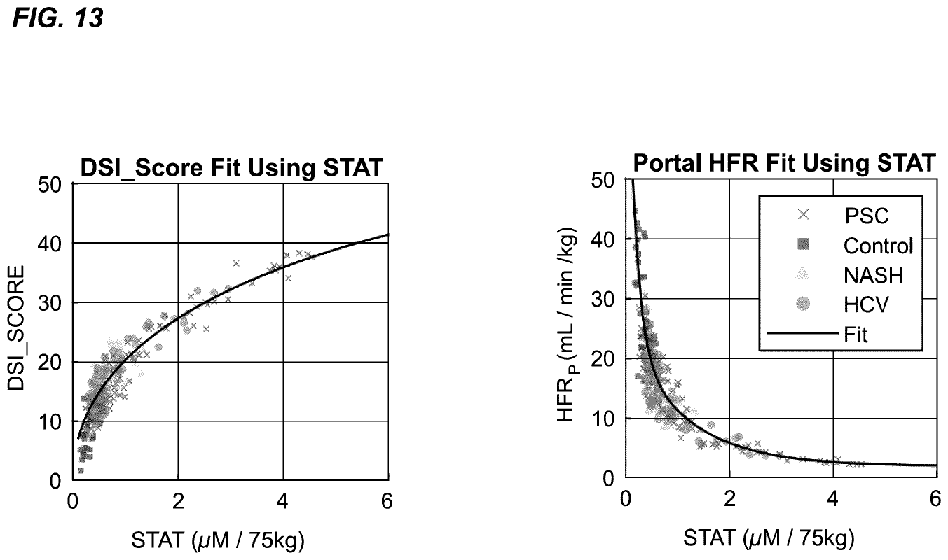

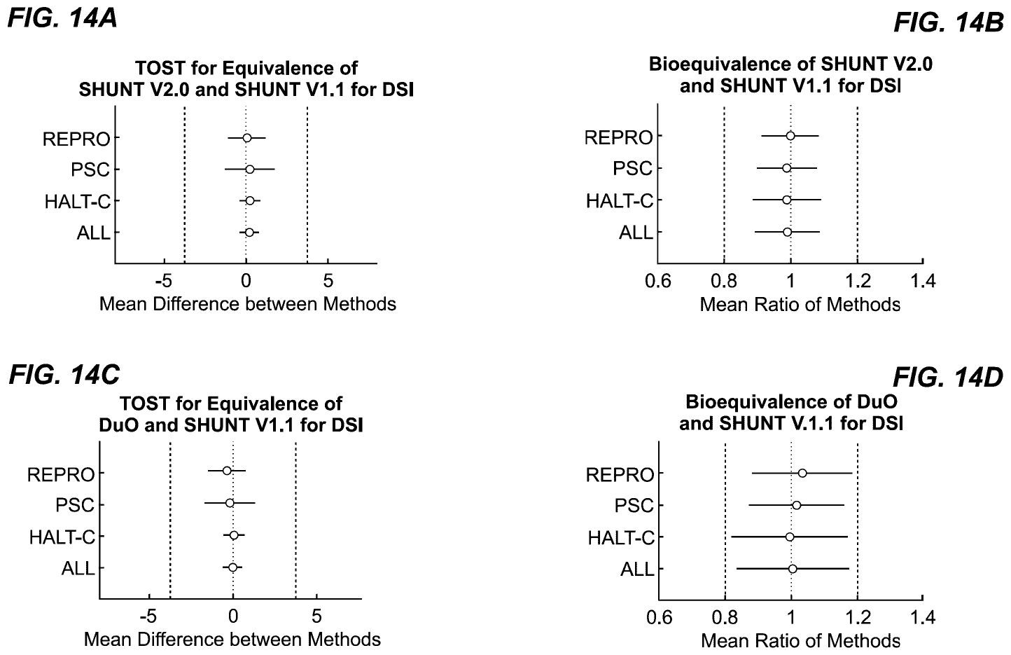

DuO. DuO was equivalent to the reference method for DSI (97%), Portal HFR (99%), and Hepatic Reserve (95%)—but not SHUNT % (85%), and Systemic HFR (82.5%). The DSI from DuO showed excellent agreement with the reference method (FIG. 16A-16B), with Deming regression line near identity (R²=0.95, slope=1.01, intercept=−0.16). The DSI from DuO was found to be statistically equivalent, not different, and bioequivalent to SHUNT V1.1 albeit with slightly larger 90% confidence intervals than those for SHUNT V2.0 (FIG. 14D). SHUNT % estimated by DuO had modest agreement to the reference method (data not shown) (R²=0.86, slope=0.87, intercept=0.004). DuO demonstrated excellent agreement for Hepatic Reserve (FIG. 18A-18) (R²=0.97, slope=1.01, intercept=0.01). Additionally Hepatic Reserve from DuO was found to be statistically equivalent, not different, and bioequivalent to SHUNT V1.1 by the TOST and bioequivalence methods (FIG. 19A-192).

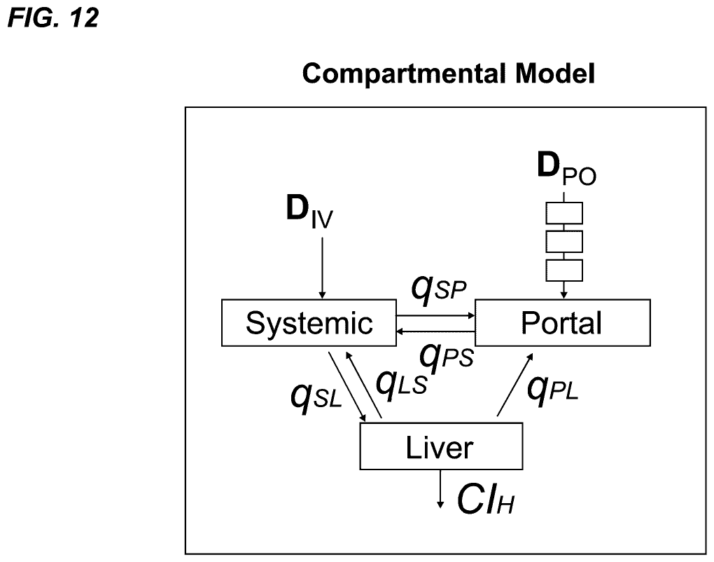

STAT. For the STAT test, Portal HFR (98%) was equivalent to the reference method, but not DSI (85%). The DSI estimated by the STAT test (FIG. 17A-179) had a relatively low bias (−0.93 DSI units), but its overall agreement with the reference method was modest in terms of correlation (R²=0.89, slope=0.92, intercept=2.52).

Equivalency to SHUNT V1.0

A secondary analysis using SHUNT V1.0 as the reference method was performed. Results are summarized in Table 22.

TABLE 22
Equivalence of next generation test methods to SHUNT V1.0 in terms of
the number of tests (percent of tests) within the equivalence bounds.

| | | REPRO | | PSC | | HALT-C | | Combined | |
|---|---|---|---|---|---|---|---|---|---|
| Parameter | Test Version | (N = 144) | | (N = 133) | | (N = 217) | | (N = 494) | |
| DSI | SHUNT V1.1 | 144 | (100) * | 133 | (100) * | 648 | (99) * | 925 | (99) * |
| | SHUNT V2.0 | 143 | (99) * | 132 | (99) * | 643 | (98) * | 918 | (99) * |
| | DuO | 143 | (99) * | 131 | (99) * | 613 | (94) | 887 | (95) * |
| | STAT | 114 | (79) | 119 | (89) | 585 | (90) | 818 | (88) |
| SHUNT % (%) | SHUNT V1.1 | 144 | (100) * | 130 | (98) * | 644 | (99) * | 918 | (99) * |
| | SHUNT V2.0 | 144 | (100) * | 125 | (94) | 627 | (96) * | 896 | (96) * |
| | DuO | 129 | (90) | 100 | (75) | 550 | (84) | 779 | (84) |
| | STAT | NA | | NA | | NA | | NA | |
| Hepatic Reserve | SHUNT V1.1 | 144 | (100) * | 133 | (100) * | 639 | (98) * | 916 | (98) * |
| (%) | SHUNT V2.0 | 144 | (100) * | 131 | (99) * | 597 | (91) | 872 | (94) |
| | DuO | 143 | (99) * | 128 | (96) * | 568 | (87) | 839 | (90) |
| | STAT | NA | | NA | | NA | | NA | |
| Portal HFR | SHUNT V1.1 | 144 | (100) * | 133 | (100) * | 653 | (100) * | 930 | (100) * |
| (mL min⁻¹ kg⁻¹) | SHUNT V2.0 | 144 | (100) * | 132 | (99) * | 644 | (99) * | 920 | (99) * |
| | DuO | 144 | (100) * | 132 | (99) * | 644 | (99) * | 920 | (99) * |
| | STAT | 138 | (96) * | 131 | (99) * | 640 | (98) * | 909 | (98) * |
| | SHUNT V1.1 | 139 | (97) * | 127 | (95) * | 646 | (99) * | 912 | (98) * |
| Systemic HFR | SHUNT V2.0 | 139 | (97) * | 126 | (95) * | 645 | (99) * | 910 | (98) * |
| (mL min⁻¹ kg⁻¹) | DuO | 118 | (82) | 106 | (80) | 600 | (92) | 824 | (89) |

| | | | | |
|---|---|---|---|---|
| STAT | NA | NA | NA | NA |

* Above acceptable limit (≥95%)

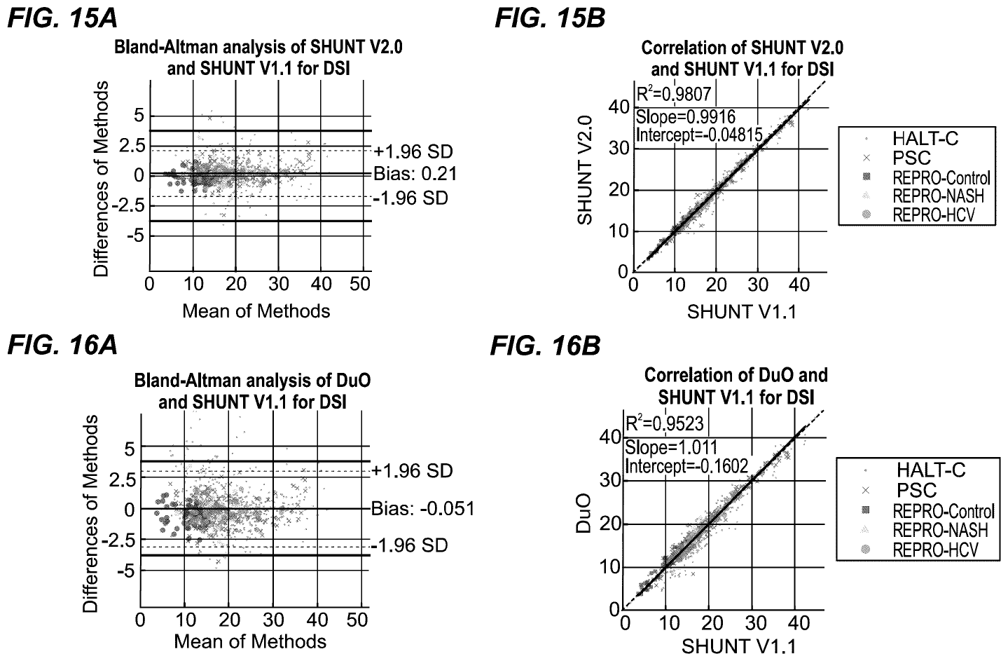

SHUNT V1.1 was equivalent to SHUNT V1.0 with >98% of values within the equivalence bounds for DSI, SHUNT %, Hepatic Reserve, Portal HFR, and Systemic HFR. The DSI measured by SHUNT V1.1 had excellent agreement to SHUNT V1.0, with low bias (0.21 DSI units) and Deming regression line approaching identity (FIG. 20A-20B) (R²=0.99, slope=1.00, intercept=−0.15). Relative to SHUNT V1.1 as the reference method, simplified test versions had demonstrated levels of equivalency with SHUNT V1.0 as the reference method.

This example used several methods to confirm the equivalency of DSI from the DuO cholate liver function test method and SHUNT V2.0 tests to DSI from the more complicated SHUNT test (V1.0/1.1). Table 23 summarizes the key findings from this study as well as the findings from the reproducibility analysis above.

Table 23 shows a summary of equivalence testing of simplified methods (SHUNT V2.0 and DuO) relative to SHUNT V1.1 and reproducibility by whether the criteria were met (Y=yes, N=no). For equivalency, the criteria were ≥95% of tests within equivalence bounds, two one-sided t-test (TOST), and bioequivalence (BE). For reproducibility, the criterion was intraclass correlation coefficient (CC)>0.7.

TABLE 23
Summary of equivalence testing of simplified methods (SHUNT
V2.0 and DuO) relative to SHUNT V1.1 and reproducibility

| | | | | | Repro- |
|---|---|---|---|---|---|
| | | Equivalency to SHUNT V1.1 | | | ducibility |
| Parameter | Test Version | ≥95% | TOST | BE | ICC > 0.7 |
| DSI | SHUNT V2.0 | Y | Y | Y | Y |
| | DuO | Y | Y | Y | Y |
| SHUNT % | SHUNT V2.0 | Y | Y* | Y | Y |
| | DuO | N | Y* | N | Y |
| Hepatic | SHUNT V2.0 | Y | Y | Y | Y |
| Reserve | DuO | Y | Y | Y | Y |
| Portal HFR | SHUNT V2.0 | Y | Y* | N | Y |
| | DuO | Y | Y* | N | Y |
| Systemic | SHUNT V2.0 | Y | Y | Y | Y |
| HFR | DuO | N | Y* | N | Y |

*Statistically equivalent and statistically different (i.e., the difference is not meaningful in this application)

DSI from SHUNT V1.0 has been associated with a number of relevant laboratory tests, clinical models, clinical complications, and clinical outcomes. By demonstrating equivalency to DSI from SHUNT (V1.0/1.1) as well as high within-individual reproducibility, DSI from DuO or SHUNT V2.0 is likely to also demonstrate the same associations with clinical endpoints.

It has been demonstrated that a two sample, oral dose, cholate challenge test (DuO) and a simplified version of the cholate SHUNT liver function test (SHUNT V2.0) provided equivalent results to the original cholate SHUNT test in less time and requiring fewer blood samples. Cholate SHUNT V2.0 and DuO liver function test methods were found to be equivalent to the original cholate SHUNT test for Disease Severity Index (DSI), with >99% and >96% of tests falling within equivalence bounds, respectively. Both DuO and SHUNT V2.0 met the additional equivalency criteria set forth in FDA guidance documents, two one-sided t-tests and bioequivalence. DuO and cholate SHUNT V2.0 are easier to administer and less invasive than the original SHUNT test and have the potential to be more widely accepted by healthcare providers and patients.

### Example 4. Prediction of Clinical Outcome in Primary Sclerosing Cholangitis

In this example liver function and portal-systemic shunting was quantified in primary sclerosing cholangitis (PSC) using the cholate SHUNT V1.1 test and simplified tests DuO cholate test and cholate SHUNT V2.0 based on a compartmental model as described herein. The test results were compared to standard laboratory tests and clinical models in the prediction of clinical outcome.

Aims: Two aims of this study were to determine whether simplified cholate tests DuO and SHUNT V2.0 could predict clinical outcome in PSC and to measure reproducibility of the tests.

Methods: The study included 47 patients spanning the clinical spectrum of PSC; 46 patients were retested at baseline for reproducibility and 40 patients were followed prospectively for clinical outcomes were retested after 1 year.

Cholate SHUNT Test Administration included 13C-cholate injected by IV and d4-cholate administered orally to the subjects. Blood was sampled at 0, 5, 20, 45, 60, 90 min. for serum cholate. The simplified cholate test versions included cholate SHUNT V1.1, cholate SHUNT V2.0, and DuO cholate tests. The SHUNT V1.1 test included all 13C- and d4-CA concentrations except for the 5 min. data points. The cholate SHUNT V2.0 included 13C- and d4-CA concentrations at 20 and 60 min. The DuO cholate test included only d4-CA concentrations at 20 and 60 min.

Test parameters included liver disease severity index (DSI), portal-systemic shunt (SHUNT %), Hepatic Reserve (HR), and Portal Hepatic Filtration Rate (HFRP). Statistical analysis included three subgroups of PSC progressors characterized from age-related degree of hepatic impairment: slow (n=28), moderate (n=16), and rapid (n=3).

Reproducibility was determined by intraclass correlation coefficient (ICC), minimum detectable difference (MDD), coefficient of variation (% CV); an ICC >0.7 was considered acceptable.

AUROCs were compared across test versions for prediction of clinical outcome (new clinical decompensation, liver-related death, liver transplantation), using the DeLong method.

Logistic regression of baseline SHUNT % for portal hypertension and varices was calculated.

Results: Cholate test parameters from DuO including liver disease severity index (DSI), portal-systemic shunt (SHUNT %), Hepatic Reserve (HR), and Portal Hepatic Filtration Rate (HFRP), laboratory and clinical test scores for PSC slow progressors (n=28), intermediate and rapid progressors (n=19) and Group t-test p values are shown in Table 24.

TABLE 24
Cholate Test Parameters from DuO cholate test,
Clinical and Laboratory Test Scores for PSC Slow
Progressors and Intermediate/Rapid Progressors

| | | |
|---|---|---|
| Slow | Intermed./Rapid | |
| Progressors | Progressors | Group t-test (p |
| (N = 28) | (N = 19) | value) |

DuO cholate test

| | | | |
|---|---|---|---|
| SHUNT % (%) | 25.0 ± 4.1 | 46.3 ± 14.7 | <0.001 |
| DSI | 14.4 ± 2.7 | 25.0 ± 6.8 | <0.001 |
| Hepatic Reserve (%) | 92.1 ± 6.0 | 64.7 ± 17.5 | <0.001 |
| Portal HFR | 18.5 ± 4.8 | 8.2 ± 4.1 | <0.001 |
| (mL/min/kg) | | | |

Labs and Clinical Scores

| | | | |
|---|---|---|---|
| Alk. Phos. (IU/L) | 128 ± 83 | 387 ± 353 | 0.0006 |
| GGT (IU/L) | 153 ± 141 | 341 ± 343 | 0.0152 |
| AST (IU/L) | 40 ± 15 | 88 ± 51 | 0 |
| ALT (IU/L) | 46 ± 25 | 80 ± 59 | 0.0086 |
| Bilirubin (mg/dL) | 1.16 ± 0.46 | 2.99 ± 2.91 | 0.0023 |
| INR | 1.05 ± 0.28 | 1.29 ± 0.80 | 0.15 |
| Albumin (g/dL) | 3.87 ± 0.31 | 3.31 ± 0.42 | <0.0001 |
| Creatinine (mg/dL) | 0.95 ± 0.17 | 0.82 ± 0.22 | 0.0448 |
| Platelets (nL⁻¹) | 201 ± 78 | 160 ± 103 | 0.13 |
| APRI | 0.58 ± 0.31 | 1.92 ± 1.52 | 0.0001 |
| FIB4 | 1.91 ± 1.16 | 4.05 ± 3.17 | 0.0029 |
| MELD score | 8.11 ± 2.23 | 11.58 ± 5.44 | 0.0039 |
| Child-Pugh Score | 5.25 ± 0.59 | 7.1 ± 1.8 | <0.0001 |
| CP A | 26 (93%) | 9 (47%) | — |
| CP B | 2 (7%) | 8 (42%) | — |
| CP C | 0 | 2 (11%) | — |
| Mayo PSC Risk | 0.31 ± 0.52 | 1.31 ± 0.94 | <0.0001 |
| Score | | | |
| Splenomegaly | 4 (14%) | 13 (68%) | 0.0002 |
| Varices | 3 (11%) | 12 (63%) | 0.0002 |
| Decompensation | 1 (3.5%) | 7 (37%) | 0.003 |

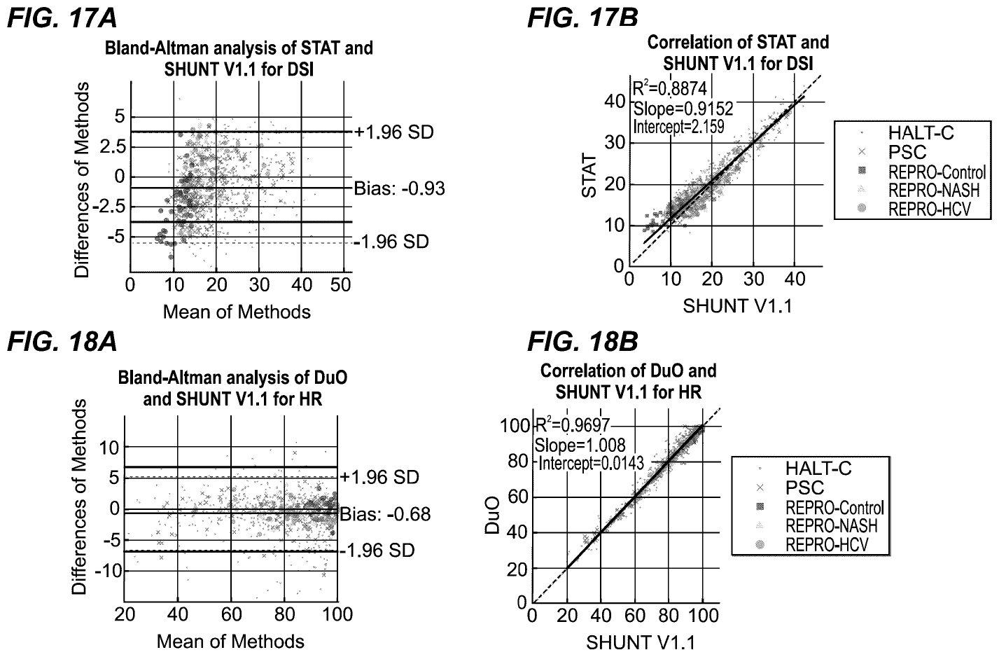

A graph of PSC Progressor Groups showing subject age vs. DSI value based on DSI from DuO is shown in FIG. 21A. A graph of PSC Progressor Groups showing subject age vs. SHUNT % value based on SHUNT % calculated from DuO is shown in FIG. 21B.

PSC rapid progressors were characterized by high SHUNT % at relatively young age. Intermediate/rapid progressors had worse DuO cholate test parameters, worse laboratory tests, and were more likely to experience clinical outcome compared to slow progressors, as shown in Table 24 and FIG. 21B.

Prediction of clinical outcome and reproducibility for DSI and SHUNT % calculated by SHUNT V1.1, SHUNT V2.0, and DuO cholate tests are shown in Table 25.

TABLE 25
Prediction of Clinical Outcome and Reproducibility of DSI
and SHUNT % calculated by SHUNT V1.1, SHUNT V2.0, or DuO

| | | Prediction of | Reproducibility | | | |
|---|---|---|---|---|---|---|
| | | clinical outcome | | | | p- |
| Parameter | Version | AUROC (95% CI) | % CV | MDD | ICC (95% CI) | value |
| DSI | SHUNT V1.1 | 0.81 (0.55-0.92) | 9.21% | 2.68 | 0.91 (0.84-0.95) | <0.001 |
| | SHUNT V2.0 | 0.80 (0.59-0.93) | 10.77% | 3.19 | 0.89 (0.81-0.94) | <0.001 |
| | DuO | 0.81 (0.62-0.92) | 10.33% | 3.02 | 0.89 (0.82-0.94) | <0.001 |
| SHUNT % | SHUNT V1.1 | 0.89 (0.73-0.96) | 10.51% | 5.88% | 0.91 (0.85-0.95) | <0.001 |
| | SHUNT V2.0 | 0.85 (0.71-0.94) | 11.41% | 6.86% | 0.90 (0.82-0.94) | <0.001 |
| | DuO | 0.83 (0.62-0.94) | 9.79% | 5.57% | 0.91 (0.84-0.95) | <0.001 |

DSI and SHUNT % had excellent within-individual reproducibility and were the strongest predictors of new clinical decompensation, liver-related death, or liver transplantation (n=13) with no significant differences between Test versions, as shown in Table 25.

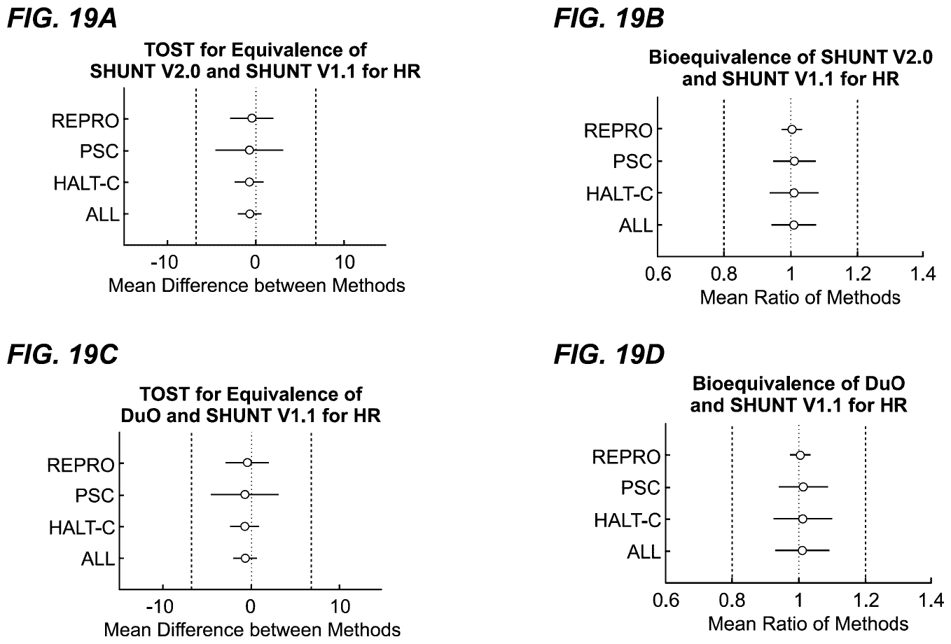

SHUNT % is linked to baseline features of portal hypertension (varices, splenomegaly, platelets <140,000) as shown in FIG. 22A, and varices as shown in FIG. 22B.

Conclusions: Cholate test parameters of liver function and physiology correlate with laboratory and clinical evidence of PSC disease severity, identify progressor groups, and predict risk for clinical outcome. There were no significant differences between test versions in terms of predicting clinical outcome and reproducibility. DuO and SHUNT V2.0 cholate tests are easier to administer and less invasive, thus, having the potential to be more widely accepted by care providers administering the test and by patients receiving the test.

### Example 5. Simplified Cholate Tests Measure Reduction in Risk for Clinical Events in Compensated NASH Cirrhosis Subjects Treated with Resmetirom

Nonalcoholic steatohepatitis (NASH) is a progressive liver disease with no approved treatment. In general, about 25% of patients with NAFLD have NASH defined as presence of ≥5% hepatic fat (steatosis) in combination with hepatocyte injury (ballooning) and inflammation.

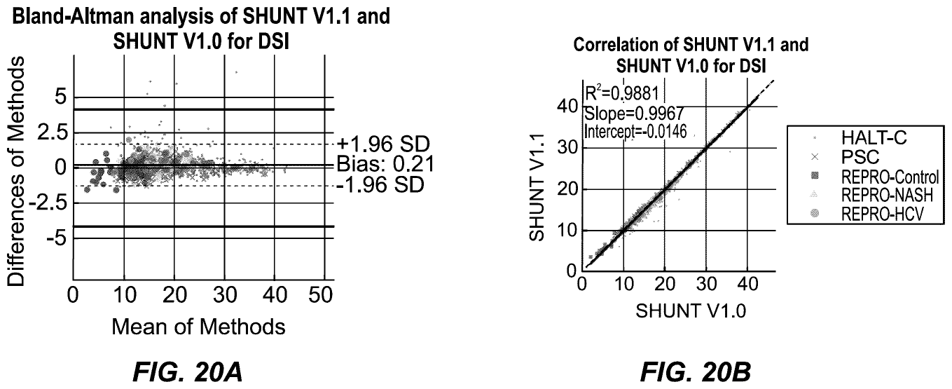

MAESTRO-NAFLD-1 (NCT04197479) was a 52-week Phase 3 trial to evaluate the safety and tolerability of resmetirom, a thyroid hormone receptor-β agonist being studied for the treatment of NASH. (Harrison et al., Lancet 2019, 394:2012-24). MAESTRO-NAFLD-1 included an open-label active resmetirom treatment arm in patients with well-compensated (Child-Pugh A [CP-A]) NASH cirrhosis (FIG. 23).

The cholate SHUNT test quantifies liver function and physiology. Simplified versions (e.g., SHUNT V2.0 and DuO cholate tests) require fewer blood samples and shorter testing time.

The objective of this study was to determine whether the simplified cholate tests SHUNT V2.0 and DuO cholate tests could detect treatment effects in MAESTRO-NAFLD-1.

Methods: Subjects (n=34) with compensated NASH cirrhosis underwent baseline testing and subsequent retesting at 28 and 48 weeks. Eligibility included at least 3 metabolic risk factors and NASH cirrhosis diagnosed by liver biopsy or accepted criteria

The cholate SHUNT test included intravenous 13C-CA and oral d4-CA administration. Blood was sampled at 0, 5, 20, 45, 60, and 90 minutes for serum cholate concentrations. AUCs were calculated by a compartmental model (McRae et al., 2023 Translational Res. 252:53-63). For SHUNT V2.0: IV and oral data at 20 and 60 minutes were employed. For DuO: only oral data at 20 and 60 minutes were employed.

Risk ACE was calculated for each subject from the baseline and weeks 28 and 48 disease severity index (DSI). A Poisson model (Risk ACE) estimated the annual clinical event rate based on 220 subjects with 52 clinical events from the HALT-C Trial. The result is an event rate (clinical events per person-year).
Model A: Relationship with baseline DSI (denoted by dsi0):

Y=β₀+β₁dsi₀
Model D: Relationship with baseline DSI and change in DSI (denoted dsiΔ)

Y=β₀+β₁dsi₀+β₂dsi_{Δ}

Difference from baseline is represented by the difference of Risk ACE Model A and Model D (Table 26).

TABLE 26
Difference of RISK ACE Model A and Model D

| | Coefficient |
|---|---|
| | Value |
| Model A | |
| β₀ | −6.3 |
| β₁ | 0.15 |
| Model D | |
| β₀ | −7.201 |
| β₁ | 0.173 |
| β₂ | 0.139 |

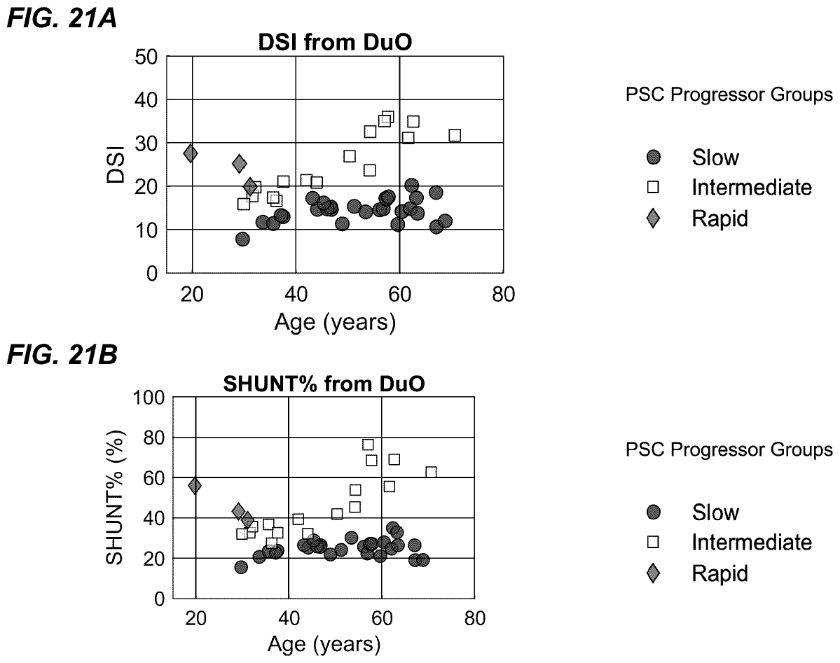

Cholate test versions DuO and SHUNT V2.0 significantly simplify test administration, reducing blood samples by two-thirds and test time by one-third as shown in FIGS. 24A and 24B showing intravenous and oral cholate clearance curves. Only cholate serum values at 20 and 60 min post administration were required.

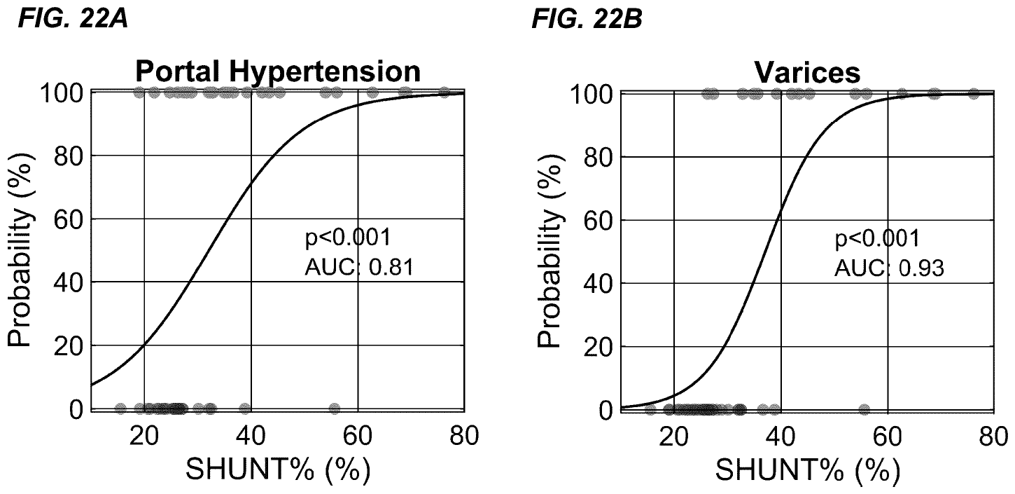

RISK ACE calculated by DuO cholate test as change from baseline at 28 and 48 weeks of Resmetirom is shown in FIGS. 25A and 25B, respectively.

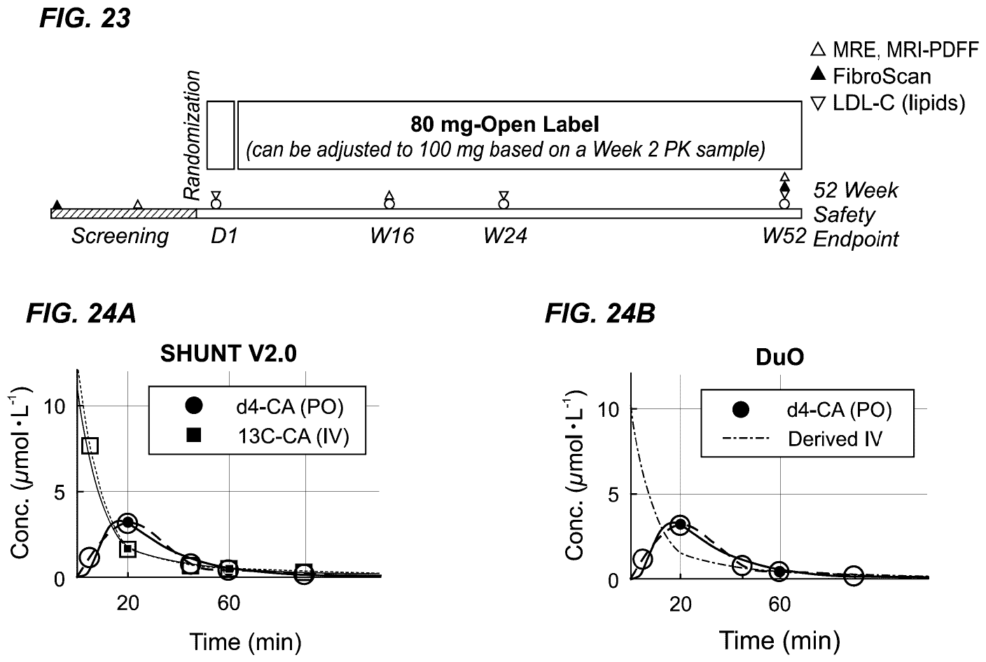

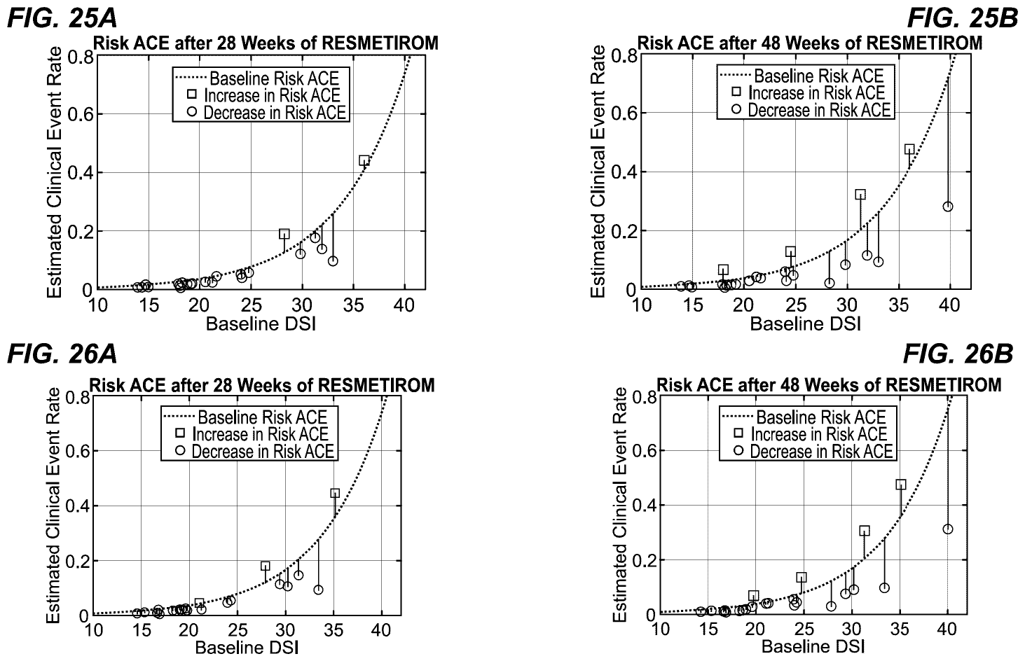

RISK ACE calculated by cholate SHUNT V2.0 test as change from baseline at 28 and 48 weeks of Resmetirom is shown in FIGS. 26A and 268, respectively.

RISK ACE calculated by SHUNT V2.0 and Duo cholate test versions as change from baseline at weeks 28 and 48 are shown in Table 27.

TABLE 27
RISK ACE calculated by SHUNT V2.0
and DuO Cholate Test versions

| | Baseline - Week 28 | | Baseline - Week 48 | |
|---|---|---|---|---|
| | Δ Risk ACE | | Δ Risk ACE | |
| Test Version | (SD) | P Value | (SD) | P Value |
| SHUNT V2.0 | −0.0174 | 0.1233 | −0.0325 | 0.1605 |
| | (0.0497) | | (0.1073) | |
| DuO | −0.0182 | 0.0407 | −0.0355 | 0.1222 |
| | (0.0401) | | (0.1059) | |

Risk ACE from DuO decreased with resmetirom treatment in 21 of 23 subjects, with significant decrease in the mean at 28 weeks as shown in Table 27. At 48 weeks, Risk ACE decreased in 19 of 23 subjects (−0.0355, p=0.1222). SHUNT V2.0 showed similar reductions in Risk ACE at 28 weeks (V2.0: −0.0170, p=0.1145) and 48 weeks (V2.0: −0.0325, p=−0.1605).

Conclusions: The simplified cholate tests measured a reduction in estimated clinical event rate after 28 weeks of resmetirom. The DuO cholate test provides a sensitive and interpretable metric of risk for All Clinical Events in monitoring patients. DuO and SHUNT V2.0 are easier to administer and less invasive, thus, having the potential to be more widely accepted by care providers administering the test and by patients receiving the test.

Clauses
Clause 1. A method for assessing liver function in a subject having or suspected of having or contracting a liver disease, comprising
obtaining blood or serum sample concentration data of an orally administered distinguishable cholate compound collected from a subject at two time points after oral administration;
measuring the area under the curve of the blood or serum concentrations of the orally administered distinguishable cholate compound (AUCoral) in the subject comprising
   simulating a full oral clearance curve using a compartmental model of oral cholate clearance, the compartmental model comprising body mass index (BMI), body weight (BW), and optionally hematocrit (Hct) input values in the subject, and
   calculating the area comprising trapezoidal numerical integration to obtain the AUCoral; and
calculating one or more indices of hepatic disease in the subject using the AUCoral, wherein the one or more indices is associated with liver function in the subject.
Clause 2. The method of clause 1, wherein the obtaining concentration data of the orally administered distinguishable cholate compound at the two time points comprises
receiving first and second blood or serum samples that had been collected from the subject at first and second time points following a single oral dose of a first distinguishable cholate compound; and
analyzing the samples to obtain the oral concentration data at the first and second time points, optionally
wherein the blood or serum samples had been collected within about 180 minutes, 120 minutes, 90 minutes, or within about 75 minutes, after the oral administration.
Clause 3. The method of clause 2, wherein the first and second blood or serum samples had been collected from the subject between at least about 5 min to about 75 min, 10 min to 70 min, 20 min to 60 min, 25 min to 55 min, 30 min to 50 min, 35 to 45 min, or about 40 min apart.
Clause 4. The method of claim 2 or 3, wherein the first and second blood or serum samples had been collected from the subject at about 20 min and about 60 min following the oral administration, respectively.
Clause 5. The method of clause 1, wherein the obtaining concentration data of the orally administered distinguishable cholate compound at the two time points comprises
receiving a single blood or serum sample that had been collected from the subject following administration of a first oral dose of a first distinguishable cholate compound and a second oral dose of a second distinguishable cholate compound to the subject, and
analyzing the single sample to obtain the oral concentration data of the first distinguishable cholate compound and second distinguishable cholate compound at the two time points, optionally
wherein the single blood or serum sample had been collected from the subject within about 180 minutes, 120 minutes, 90 minutes, or within about 75 minutes, after the first oral dose administration.
Clause 6. The method of clause 5, wherein the first and second oral doses had been administered to the subject between at least about 5 min to about 75 min, 10 min to 70 min, 20 min to 60 min, 25 min to 55 min, 30 min to 50 min, 35 to 45 min, or about 40 min apart.
Clause 7. The method of clause 5 or 6, wherein the single sample had been collected from the subject at about 20 min after the second oral dose and simultaneously at about 60 min after the first oral dose.
Clause 8. The method of any one of clauses 1 to 7, further comprising
estimating an area under the curve of blood or serum concentrations of an intravenously administered distinguishable cholate compound (AUCiv); and
calculating the one or more indices of hepatic disease in the subject using the AUCoral and AUCiv values.
Clause 9. The method of clause 8, wherein the estimating the AUCiv comprises a linear regression model, optionally wherein the linear regression model comprises equation 11A:

AUC_{IV}=β₀+β_{BW}·BW+β_{PO,20}·C_{PO,20}+β_{PO,60}·C_{PO,60}+β_{HFR,P}·HFR_{P}, Eqn. 11A

wherein
β₀ is an intercept coefficient, optionally wherein the intercept coefficient is 161.972;
β_{BW} is a body weight coefficient, optionally wherein the body weight coefficient is 0.6459;
β_{PO,20} is an orally administered distinguishable cholate concentration coefficient at a first time point, optionally wherein the β_{PO,20} is 16.9249;
C_{PO,20} is the orally administered distinguishable cholate concentration at the first time point;
β_{PO,60} is an orally administered distinguishable cholate concentration coefficient at a second time point, optionally wherein the β_{PO,60} is 89.2405;
β_{PO,60} is an orally administered distinguishable cholate concentration at the second time point; and
β_{HFR,P} is a portal HFR coefficient, optionally wherein the portal HFR coefficient is −0.4755.
Clause 10. The method of clause 8, wherein the estimating the AUCiv comprises
obtaining blood or serum sample concentration data of an intravenously administered third distinguishable cholate compound in one of the samples that had been collected from the subject, and
exponential fitting the intravenous concentration data to a systemic cholate clearance curve comprising fast, moderate, and slow phases of clearance over at least about 180 min after the iv administration of the intravenous dose.
Clause 11. The method of clause 10, wherein the third distinguishable cholate had been intravenously administered simultaneously with the first oral dose, or at least about 5 min to about 75 min, 10 min to 70 min, 20 min to 60 min, 25 min to 55 min, 30 min to 50 min, 35 to 45 min, or about 40 min after the first oral dose.
Clause 12. The method of clause 10 or 11, wherein the blood or serum sample collected at one time point had been collected within about 90 minutes or less, 75 minutes or less, 60 minutes or less, 45 minutes or less, 30 minutes or less, or about 20 minutes after the intravenous administration.
Clause 13. The method of any one of clauses 10 to 12, wherein the fitting to systemic cholate clearance curve fast phase (Y₀) is calculated according to equation 20:

Y₀=C₀·e^{-k}^{_{fast}}^{·t} Eqn. 20,

wherein
t=time (0 to 20 min);
C₀ is the initial concentration of intravenously administered distinguishable cholate compound,
C₂₀ is the measured 20-minute concentration of intravenously administered distinguishable cholate compound; and
k_{fast} is the rate of elimination in the fast phase estimated by equation 18:

$${k}_{fast} = \frac{ln ( {C}_{0} ) - ln ( {C}_{20} )}{20} , \qquad Eqn . 18$$

optionally wherein
C₀ is estimated according to equation 17;

$${C}_{0} = \frac{{D}_{IV}}{{V}_{d} \cdot BW} , \qquad Eqn . 17$$

wherein
D_{IV} is the intravenous dose of third distinguishable cholate;
BW is subject body weight (kg); and
   Vd is the volume of distribution (V_{d}) in L per kg body weight, calculated according to equation 16A:

$$T P V = {V}_{d} = \frac{0.07}{\sqrt{B M I / 22}} \cdot ( 1 - Hct ) , \qquad Eqn . 16 A$$

wherein TPV is total plasma volume, BMI is body mass index, and Hct is hematocrit in the subject.
Clause 14. The method of any one of clauses 10 to 13, wherein the fitting to systemic cholate clearance curve moderate phase (Y₁) is calculated according to equation 21:

$${Y}_{1} = \frac{{C}_{20}}{{e}^{- {k}_{{mod}^{\cdot 20}}}} \cdot {e}^{- {k}_{{mod}^{\cdot t}}} , \qquad Eqn . 21$$

t=time (20-45 min);
k_{mod} is the rate of elimination in the moderate phase estimated by equation 19:

k_{mod}=β_{0,mod}+β_{kfast}·k_{fast}+β_{IV,20}·C_{IV,20}+β_{PO,20,mod}·C_{PO,20}+β_{PO,60,mod}·C_{PO,60} Eqn. 19,

wherein
β_{0,mod} is the intercept coefficient, optionally wherein the intercept coefficient is 0.0268;
β_{kfast} is the k_{fast} coefficient, optionally wherein the k_{fast} coefficient is 0.546;
β_{IV,20} is the coefficient of the intravenous distinguishable cholate concentration at the 20 min time point; optionally wherein β_{IV,20} is 0.0045;
β_{PO,20,mod} is the coefficient of the oral distinguishable cholate concentration at the 20 min time point, optionally wherein β_{PO,20,mod} is −0.0007;
β_{PO,60,mod} is the coefficient of the oral distinguishable cholate concentration at the 60 min time point, optionally wherein β_{PO,60,mod} is −0.0052.
Clause 15. The method of any one of clauses 10 to 14, wherein the fitting to systemic cholate clearance curve slow phase (Y₂) is calculated according to equation 22:

$${Y}_{2} = \frac{{C}_{45}}{{e}^{- {k}_{{slow}^{\cdot 45}}}} \cdot {e}^{- {k}_{{slow}^{\cdot t}}} , \qquad Eqn . 22$$

wherein
t=time (45-180 min);
C₄₅ is the estimated 45-minute concentration of intravenously administered distinguishable cholate; and
k_{slow} is the rate of elimination in the slow phase estimated by a mean value from a multiplicity of CLD patients, optionally wherein k_{slow} is 0.018 min⁻¹.
Clause 16. The method according to any one of clauses 10 to 15, wherein the areas under each of the three exponential curve fits are calculated by trapezoidal numerical integration and summed to estimate the AUC_{IV}.
Clause 17. The method of any one of clauses 1 to 16, wherein the compartmental model of oral clearance comprises estimating
compartment volumes of a plurality of compartments in the subject; and
flow parameters between the plurality of compartments in the subject.
Clause 18. The method of clause 17, wherein the compartmental model further comprises estimating cholate binding and dose administration in the subject.
Clause 19. The method of clause 17 or 18, wherein the plurality of compartments in the subject comprises systemic, portal, and liver compartments.
Clause 20. The method of clause 18 or 19, wherein the estimating compartment volumes of the plurality of compartments comprises
estimating the systemic compartment volume (V_{S}), the portal compartment volume (V_{P}), and the liver compartment volume (V_{L}) in the subject, optionally each compartment volume in liters (L).
Clause 21. The method of clause 20, wherein the systemic compartment volume (V_{S}) is estimated according to Equation 4A:

V_{S}=TBV·(1−Hct) Eqn. 4A,

wherein
TBV is total blood volume in the subject according to equation 4:

$$T B V = \frac{0.07 \cdot B W}{\sqrt{B M I / 22}} , \qquad Eqn . 4$$

wherein
   BMI is body mass index (kg/m²) in the subject, and
   BW is body weight (kg) in the subject; and
      Hct is the hematocrit in the subject.
Clause 22. The method of clause 20 or 21, wherein the portal compartment volume (V_{P}) is estimated according to Equation 4B:

V_{P}=0.25·V_{S} Eqn. 4B.
Clause 23. The method of any one of clauses 20 to 22, wherein the liver compartment volume (V_{L}) is estimated according to Equation 5A:

V_{L}=(0.275·22.46·BW·d_{L}·fₚₗₐₛₘₐ)/1000 Eqn. 5A,

wherein d_{L} is assumed Liver tissue density of 1.06 g·mL⁻¹.
Clause 24A. The method of any one of clauses 17 to 23, wherein the estimating the flow parameters between the plurality of compartments comprises a system of first-order ordinary differential equations 1 to 3:

$$\frac{{dC}_{S}}{dt} = \frac{1}{{V}_{S}} ( - [ {q}_{SP} + {q}_{SL} ] {C}_{S} + {q}_{LS} \cdot {C}_{L} ) ; \qquad Eqn . 1 A$$

$$\frac{{dC}_{P}}{dt} = \frac{1}{{V}_{P}} ( {D}_{PO} + {q}_{SP} \cdot {C}_{S} - {q}_{PL} \cdot {C}_{P} ) ; and \qquad Eqn . 2 A$$

$$\frac{{dC}_{L}}{dt} = \frac{1}{{V}_{L}} ( - [ {Cl}_{H} \cdot {V}_{L} + {q}_{LS} ] {C}_{L} + {q}_{PL} \cdot {C}_{P} + {q}_{SL} \cdot {C}_{S} ) , \qquad Eqn . 3$$

V is the volume of each of the each of the systemic (V_{S}), portal (V_{P}), and liver (V_{L}) compartments, C is the concentration of cholate in each of the systemic (C_{S}), portal (C_{P}), and liver (C_{L}) compartments, q is the flow rate between compartments, and Cl_{H} is the hepatic clearance.
Clause 24B. The method of any one of clauses 17 to 23, wherein the estimating the flow parameters between the plurality of compartments comprises a system of first-order ordinary differential equations 1B, 2B, and 3:

$$\frac{{dC}_{S}}{dt} = \frac{1}{{V}_{S}} ( - [ {q}_{SP} + {q}_{SL} ] {C}_{S} + {q}_{LS} \cdot {C}_{L} + {q}_{PS} \cdot {C}_{P} ) ; \qquad Eqn . 1 B$$

$$\frac{{dC}_{P}}{dt} = \frac{1}{{V}_{P}} ( {D}_{PO , rate} + {q}_{SP} \cdot {C}_{S} - {q}_{PL} \cdot {C}_{P} - {q}_{PS} \cdot {C}_{P} ) ; and \qquad Eqn . 2 B$$

$$\frac{{dC}_{L}}{dt} = \frac{1}{{V}_{L}} ( - [ {Cl}_{H} \cdot {V}_{L} + {q}_{LS} ] {C}_{L} + {q}_{PL} \cdot {C}_{P} + {q}_{SL} \cdot {C}_{S} ) , \qquad Eqn . 3$$

wherein V is the volume of each of the each of the systemic (V_{S}), portal (V_{P}), and liver (V_{L}) compartments, C is the concentration of orally administered distinguishable cholate compound in each of the systemic (C_{S}), portal (C_{P}), and liver (C_{L}) compartments, q is the flow rate between compartments, and Cl_{H} is the hepatic clearance, and D_{PO,rate} is the rate of orally administered distinguishable cholate compound entering the portal compartment.
Clause 25. The method of clause 24A or B, wherein the estimating comprises estimating total hepatic inflow to the liver (Q_{L}), splanchnic arterial circulation (q_{SP}), hepatic portal venous inflow to the liver (q_{PL}), total hepatic venous return flow to systemic circulation (q_{LS}), and hepatic arterial inflow to the liver (q_{SL}) in the subject.
Clause 26. The method of clause 25, wherein the estimating total hepatic inflow to the liver (Q_{L}) comprising both hepatic arterial (q_{SL}) and portal venous (q_{PL}) inflows to the liver according to equation 6A:

Q_{L}=q_{SL}+q_{PL}, (L·min⁻¹) Eqn. 6A,
wherein
q_{SL} is rate of hepatic arterial inflow to the liver,

q_{SL}=0.25·Q_{L} (L·min⁻¹); and
q_{PL} is rate of portal venous inflow to the liver,

q_{PL}=q_{SP} (L·min⁻¹), wherein
q_{SP} is splanchnic arterial blood flow rate to abdominal intestinal organs,

q_{SP}=0.75·Q_{L,init} (L·min⁻¹), optionally wherein
the initial estimate for Q_{L} is about 1 L·min⁻¹·kg⁻¹ liver wet weight.
Clause 27. The method of clause 25 or 26, wherein the calculating total hepatic venous return flow rate to systemic circulation (q_{LS}) comprises: q_{LS}=q_{PL}+q_{SL}.
Clause 28. The method of any one of clauses 24 to 27, wherein the hepatic clearance (Cl_{H}) is estimated by equation 7A:

Cl_{H}=Q_{L}·ER Eqn. 7A,

wherein
ER is the extraction ratio in the subject and is assumed to be 0.7.
Clause 29. A method for assessing liver function in a subject having or suspected of having or contracting a liver disease, comprising
obtaining input data derived from the subject comprising blood or serum sample concentration data of an orally administered distinguishable cholate compound collected from a subject at two time points within 180 minutes after oral administration, body mass index (BMI), hematocrit (Hct), and estimated volume of distribution (Vd) in the subject;
fitting the input data to a trained function fitting neural network comprising a training algorithm to generate a multiplicity of output points for oral and intravenous distinguishable cholate clearance curves;
generating oral and IV distinguishable cholate clearance curves from the fitted data; measuring AUC_{Oral} and AUC_{IV} values for the subject comprising trapezoidal numerical integration; and
calculating one or more indices of hepatic disease in the subject using the AUCoral and/or and AUC_{IV} values wherein the one or more indices is associated with liver function in the subject.
Clause 30. A method for assessing liver function in a subject having or suspected of having or contracting a liver disease, comprising
obtaining input data derived from the subject comprising blood or serum sample concentration data of an orally administered first distinguishable cholate compound collected from a subject at two time points within 180 minutes after oral administration, blood or serum sample concentration data of an intravenously administered second distinguishable cholate compound collected from a subject at one time point within 180 minutes after intravenous administration, estimated volume of distribution (Vd) in the subject, and estimated initial intravenous distinguishable cholate concentration at 0 minutes of the intravenously administered second distinguishable cholate based on the Vd;
fitting the input data to a trained function fitting neural network comprising a training algorithm to generate a multiplicity of output points for oral and intravenous distinguishable cholate clearance curves;
generating oral and IV distinguishable cholate clearance curves from the fitted data;
measuring AUC_{Oral} and AUC_{IV} values for the subject comprising trapezoidal numerical integration; and
   calculating one or more indices of hepatic disease in the subject using the AUCoral and/or and AUC_{IV} values wherein the one or more indices is associated with liver function in the subject.
Clause 31. The method of clause 29 or 30, wherein the estimated Vd (L per kg body weight) in the subject is estimated by equation 16A:

$$T P V = {V}_{d} = \frac{0.07}{\sqrt{B M I / 22}} \cdot ( 1 - Hct ) , \qquad Eqn . 16 A$$

wherein TPV is total plasma volume, BMI is body mass index, and Hct is hematocrit in the subject.
Clause 32. The method of any one of clauses 29 to 31, wherein the neural network is configured for regression tasks and comprises a 2-layer feedforward network comprising a hidden layer and an output layer, optionally comprising a sigmoid transfer function in the hidden layer and a linear transfer function in the output layer.
Clause 33. The method of any one of clauses 29 to 32, wherein the neural network comprising a training algorithm had been trained on a training data set comprising a multiplicity of oral and intravenous distinguishable cholate clearance curves estimated by a non-compartmental minimal model (MM) from a combination of normal control subjects and chronic liver disease patients.
Clause 34. The method of any one of clauses 29 to 33, wherein the training algorithm is selected from the group consisting of a Levenberg-Marquardt backpropagation, Bayesian Regularization, BFGS Quasi-Newton, Resilient Backpropagation, Scaled Conjugate Gradient, Conjugate Gradient with Powell/Beale Restarts, Fletcher-Power Conjugate Gradient, Polak-Ribiére Conjugate Gradient, One Step Secant, Variable Learning Rate Gradient Descent, Gradient Descent with Momentum, and Gradient Descent training algorithm.
Clause 35. The method of any one of clauses 29 to 34, wherein the output points comprise 5-minute increments from 0 to 180 minutes after the oral administration, resulting in 37 timepoints for each of the oral and IV clearance curves.
Clause 36. The method of any one of clauses 30 to 35, wherein the initial intravenous distinguishable cholate compound concentration at 0 minutes is estimated comprising
estimating Vd (L per kg body weight) in the subject by equation 16A:

$$T P V = {V}_{d} = \frac{0.07}{\sqrt{B M I / 22}} \cdot ( 1 - Hct ) , \qquad Eqn . 16 A$$

dividing the IV dose by V_{d} times body weight (BW).
Clause 37. A method for assessing liver function in a subject having or suspected of having or contracting a liver disease, comprising:
(a) receiving a plurality of blood or serum samples collected from the subject following oral administration of a dose of a first distinguishable cholate compound (doseₒᵣₐₗ) to the subject and simultaneous intravenous co-administration of a dose of a second distinguishable cholate compound (doseᵢᵥ) to the subject, wherein the samples had been collected over no more than about 180 minutes, no more than about 120 minutes, or no more than about 90 minutes after administration;
(b) quantifying the concentration of the first and the second distinguishable cholate compounds; and
(c) generating individual subject oral and intravenous clearance curves from the concentration of the first and second distinguishable cholate compounds comprising using a computer algorithm curve fitting to model oral and intravenous clearance curves; and computing the area under the individualized oral and intravenous clearance curves (AUCoral) and (AUCiv), respectively, in the subject, wherein the multiplicity of samples comprise blood or serum samples collected from the subject over at least 5 time points, and
   wherein the generating individual intravenous clearance curve comprises
estimating an initial intravenous distinguishable cholate compound concentration in the subject at 0 minutes comprising
estimating Vd (L per kg body weight) in the subject by equation 16A:

$$T P V = {V}_{d} = \frac{0.07}{\sqrt{B M I / 22}} \cdot ( 1 - Hct ) ; \qquad Eqn . 16 A$$

dividing the IV dose by V_{d} times body weight (BW) to obtain the initial estimated initial intravenous distinguishable cholate concentration; and
constructing the intravenous distinguishable cholate clearance curve comprising the estimated initial intravenous concentration.
Clause 38. The method of any one of clauses 1 to 37, wherein the one or more indices of hepatic disease is selected from the group consisting of portal hepatic filtration rate (HFRp), systemic hepatic filtration rate (HFRs), cholate SHUNT, liver disease severity index (DSI), indexed hepatic reserve (HR_{indexed}), and algebraic hepatic reserve (HR_{algebraic}), in the subject.
Clause 39. The method of any one of clauses 1 to 38, wherein the calculating one or more indices of hepatic disease comprises calculating portal hepatic filtration rate (HFRp) in the subject by equation 10A:

$$H F {R}_{P} = \frac{{D}_{PO}}{{AUC}_{Oral} \cdot B W} , \qquad Eqn . 10 A$$

wherein D_{PO} is the oral dose of the orally administered distinguishable cholate, and BW is the subject body weight.
Clause 40. The method of any one of clauses 8 to 38, wherein the calculating one or more indices of hepatic disease comprises calculating systemic hepatic filtration rate (HFRs) according to equation 12:

$$H F {R}_{S} = \frac{{D}_{IV}}{{AUC}_{IV} \cdot B W} , \qquad Eqn . 12$$

wherein D_{IV} is the administered intravenous oral dose of distinguishable cholate, and BW subject body weight.
Clause 41. The method of any one of clauses 8 to 40, wherein the calculating one or more indices of hepatic disease comprises estimating cholate SHUNT (F) according to equation 13:

$$F = \frac{{AUC}_{Oral} \cdot {D}_{IV}}{{AUC}_{IV} \cdot {D}_{PO}} . \qquad Eqn . 13$$

Clause 42. The method of any one of clauses 30 to 32, wherein the calculating one or more indices of hepatic disease comprises estimating liver Disease Severity Index (DSI) according to equation 14:

$$D S I = A \cdot \sqrt{{( ln [ \frac{H F {R}_{P , max}}{H F {R}_{P}} ] )}^{2} + {( ln [ \frac{H F {R}_{S , max}}{H F {R}_{S}} ] )}^{2}} , \qquad Eqn . 14$$

wherein
HFR_{P,max} is the upper limit of portal clearance from a multiplicity of healthy controls;
HFR_{S,max} is the upper limit of clearance from a multiplicity of healthy controls; and A is a factor to scale DSI from 0 to 50.
Clause 43. The method of any one of clauses 39 to 42, wherein the calculating one or more indices of hepatic disease comprises estimating indexed Hepatic Reserve (HR_{indexed}) according to equation 15:

$$H {R}_{indexed} = 100 - A \cdot \sqrt{{( ln [ \frac{H F {R}_{P , lean}}{H F {R}_{P}} ] )}^{2} + {( ln [ \frac{H F {R}_{S , lean}}{H F {R}_{S}} ] )}^{2}} , \qquad Eqn . 15$$

wherein
HFR_{P} and HFR_{S} are indexed to lean controls minus one standard deviation (HFR_{P,lean} and HFR_{S,lean}); and A is a constant to scale HR value from 100 to 0.
Clause 44. The method of any one of clauses 39 to 42, wherein the calculating one or more indices of hepatic disease comprises estimating algebraic Hepatic Reserve (HR_{algebraic}) according to equation 15A:

HR_{algebraic}=100−2·DSI Eqn. 15A.
Clause 45. The method of any one of clauses 1 to 44, wherein the first distinguishable cholate compound is a first stable isotope labeled cholate compound, the optional second distinguishable cholate compound is a second stable isotope labeled cholate compound, and the optional third distinguishable cholate compound is a third stable isotope labeled e cholate compound.
Clause 46. The method of clause 45, wherein the first, optional second, and optional third stable isotope labeled cholate compounds are selected from d4-cholate, d2-cholate, d5-cholate, and ¹³C-cholate, optionally wherein the d4 cholate is 2,2,4,4-d4 cholate, the d5-cholate is 2,2,3,4,4-cholate, and further optionally wherein the 13C cholate is 24-¹³C-cholate.
Clause 47. The method of any one of clauses 1 to 46, wherein the subject is a human subject.
Clause 48. The method of any one of clauses 1 to 47, further comprising
comparing the one or more indices of hepatic disease in the subject to one or more cutoff values as an indicator of the relative hepatic function in the subject.
Clause 49. The method of clause 48, wherein the one or more cutoff values are derived from one or more normal healthy controls, a group of known patients, or within the subject over time.
Clause 50. The method of any one of clauses 1 to 49, further comprising
providing the one or more indices of hepatic disease to a medical professional for the purpose of developing a treatment plan in the subject.
Clause 51. The method of clause 50, wherein the one or more indices of hepatic disease is employed for a purpose selected from the group consisting of determining a need for treatment, predicting response to treatment, predicting large esophageal varices, monitoring the effectiveness of a treatment, personalized dosing of one or more drugs, and predicting risk of clinical outcome in the subject.
Clause 52. The method of any one of clauses 1 to 51, wherein the liver disease is a chronic liver disease selected from the group consisting of chronic hepatitis C (CHC), chronic hepatitis B, metabolic dysfunction-associated alcoholic liver disease (Met-ALD), alcoholic liver disease (ALD), steatotic liver disease (SLD), fatty liver disease, Alcoholic SteatoHepatitis (ASH), Alcoholic Hepatitis (AH), metabolic dysfunction-associated steatotic liver disease (MASLD), Non-Alcoholic Fatty Liver Disease (NAFLD), steatosis, metabolic dysfunction-associated steatohepatitis (MASH), Non-Alcoholic SteatoHepatitis (NASH), autoimmune liver disease, cryptogenic cirrhosis, hemochromatosis, Wilson's disease, alpha-1-antitrypsin deficiency, liver cancer, liver failure, cirrhosis, primary sclerosing cholangitis (PSC), and other cholestatic liver diseases.
Clause 53. The method of clause 51 or 52, wherein the clinical outcome is selected from the group consisting of Child-Turcotte-Pugh (CTP) progression, Model for End-stage Liver Disease (MELD) progression, variceal hemorrhage, ascites, splenomegaly, varices, large esophageal varices, portal hypertension (PHTN), hepatic encephalopathy, hepatocellular carcinoma (HCC), decompensation, or liver-related death.
Clause 54. The method of any one of clauses 50 to 53, wherein the treatment is selected from the group consisting of antiviral treatments, antifibrotic treatments, antibiotics, immunosuppressive treatments, anti-cancer treatments, ursodeoxycholic acid, farnesoid X receptor ligands, insulin sensitizing agents, interventional treatment, liver transplant, lifestyle changes, dietary restrictions, low glycemic index diet, antioxidants, vitamin supplements, transjugular intrahepatic portosystemic shunt (TIPS), catheter-directed thrombolysis, balloon dilation and stent placement, balloon-dilation and drainage, weight loss, exercise, and avoidance of alcohol.
Clause 55. The method of any one of any one of clauses 1 to 54, wherein monitoring the need for treatment in the subject comprises
determining the one or more indices of hepatic disease in the subject; and
comparing the one or more indices of hepatic disease to one or more cutoff value(s),
wherein a change in the one or more indices of hepatic disease compared to cutoff value(s) is indicative of the need for treatment in the subject.
Clause 56. The method of any one of clauses 49 to 55, wherein the group of known patients is suffering from a disease or condition selected from the group consisting of a chronic liver disease having a fibrosis stage; portal hypertension; Childs-Turcotte-Pugh (CTP) score A; CTP score B, CTP score C; Model for End-stage Liver Disease (MELD) progression score, primary sclerosing cholangitis (PSC) not listed for transplant; PSC listed for liver transplant; PSC listed for liver transplant without varices; PSC listed for liver transplant with varices; ascites; stomal bleeding; splemomegaly; varices; large varices, variceal hemorrhage; hepatic encephalopathy, decompensation; or liver disease-related death.
Clause 57. The method of clause 56, wherein the fibrosis stage is determined by a method comprising liver biopsy or elastography.
Clause 58. The method of clause 57, wherein the liver biopsy determines Ishak fibrosis score (liver biopsy) of F2 (mild portal fibrosis), F3, F4 (moderate bridging fibrosis), F5 (nodular formation and incomplete cirrhosis), or F6 (cirrhosis).
Clause 59. The method of any one of clauses 25 to 58, wherein the rate of portal venous inflow to the liver (q) differentiates healthy controls, subjects with no varices, subjects with small varices, and subjects with large varices.

### REFERENCES

1. Vajjah, P., G. K. Isbister, and S. B. Duffull, Introduction to Pharmacokinetics in Clinical Toxicology, in Computational Toxicology: Volume I, B. Reisfeld and A. N. Mayeno. Editors. 2012, Humana Press: Totowa, NJ. p. 289-312.
2. Gillespie, W. R., Noncompartmental Versus Compartmental Modelling in Clinical Pharmacokinetics. Clinical Pharmacokinetics, 1991. 20(4): p. 253-262.
3. Hirtz, J., The Fate of Drugs in the Organism, in A bibliographic survey complied by the Societé Française des Sciences et Techniques Pharmaceutique, Working Group under the chairmanship of Hirtz. 1974, Dekker New York.

4. Bassingthwaighte, J. B., et al., Compartmental Modeling in the Analysis of Biological Systems, in Computational Toxicology: Volume I, B. Reisfeld and A. N. Mayeno, Editors. 2012, Humana Press: Totowa, NJ. p. 391-438.
5. Gabrielsson, J. and D. Weiner, Non-compartmental Analysis, in Computational Toxicology: Volume I, B. Reisfeld and A. N. Mayeno, Editors. 2012, Humana Press: Totowa, NJ. p. 377-389.
6. Everson, G. T., et al., Portal-systemic shunting in patients with fibrosis or cirrhosis due to chronic hepatitis C: the minimal model for measuring cholate clearances and shunt. Alimentary Pharmacology & Therapeutics, 2007. 26(3): p. 401-410.
7. McRae, M. P., et al., Compartmental model describing the physiological basis for the HepQuant SHUNT test. Translational Research.
8. Lemmens, H. J., D. P. Bernstein, and J. B. Brodsky, Estimating blood volume in obese and morbidly obese patients. Obesity Surgery, 2006. 16(6): p. 773-6.
9. Mathew, J., P. Sankar, and M. Varacallo, Physiology, Blood Plasma, in StatPearls. 2021, StatPearls Publishing: Treasure Island (FL).
10. Eipel, C., K. Abshagen, and B. Vollmar, Regulation of hepatic blood flow: the hepatic arterial buffer response revisited. World journal of gastroenterology, 2010. 16(48): p. 6046-6057.
11. Lautt, W. W., Chapter 2, Overview, in Hepatic Circulation: Physiology and Pathophysiology. 2009, Morgan & Claypool Life Sciences: San Rafael (CA).
12. Nichols, D. G., et al., Chapter 5—Splanchnic Function and Heart Disease, in Critical Heart Disease in Infants and Children (Second Edition), D. G. Nichols, et al., Editors. 2006, Mosby: Philadelphia. p. 131-141.
13. Andersen, V., et al., The volume of the liver in patients correlates to body weight and alcohol consumption. Alcohol and Alcoholism, 2000. 35(5): p. 531-532.
14. Yu, H. C., et al., Estimation of standard liver volume for liver transplantation in the Korean population. Liver Transplantation, 2004. 10(6): p. 779-83.
15. Heinemann, A., et al., Standard liver volume in the Caucasian population. Liver Transplantation and Surgery, 1999. 5(5): p. 366-8.
16. Lautt, W. W., Hepatic vasculature: a conceptual review. Gastroenterology, 1977. 73(5): p. 1163-1169.
17. Rappaport, A. M., Hepatic blood flow: morphologic aspects and physiologic regulation. Int Rev Physiol, 1980. 21: p. 1-63.
18. Harper, D. and B. Chandler, Splanchnic circulation. BJA Education, 2016. 16(2): p. 66-71.
19. Tygstrup, N., et al., Determination of the hepatic arterial blood flow and oxygen supply in man by clamping the hepatic artery during surgery. Journal of Clinical Investigation, 1962.41(3): p. 447-454.
20. Lautt, W. W., Relationship between hepatic blood flow and overall metabolism: the hepatic arterial buffer response. Federation Proceedings, 1983. 42(6): p. 1662-6.
21. Lautt, W. W., D. J. Legare, and M. S. d'Almeida, Adenosine as putative regulator of hepatic arterial flow (the buffer response). American Journal of Physiology—Heart and Circulatory Physiology, 1985. 248(3): p. H331-H338.
22. Atkinson, A. J. and W. Kushner, Clinical Pharmacokinetics. Annual Review of Pharmacology and Toxicology, 1979. 19(1): p. 105-127.
23. Rowland, M. and T. N. Tozer, Clinical Pharmacokinetics: Concepts and Applications. Lea & Febinger Book. 1995: Williams & Wilkins.
24. Hofmann, A. F., The Continuing Importance of Bile Acids in Liver and Intestinal Disease. Archives of Internal Medicine, 1999. 159(22): p. 2647-2658.
25. Gilmore, I. T. and R. P. Thompson, Plasma clearance of oral and intravenous cholic acid in subjects with and without chronic liver disease. Gut, 1980. 21(2): p. 123.
26. O'Maille. E. R., T. G. Richards, and A. H. Short, The influence of conjugation of cholic acid on its uptake and secretion: hepatic extraction of taurocholate and cholate in the dog. The Journal of physiology, 1967. 189(2): p. 337-350.
27. Salem, F., et al., Considering Age Variation When Coining Drugs as High versus Low Hepatic Extraction Ratio. Drug Metabolism and Disposition, 2016. 44(7): p. 1099.
28. Wilkinson, G. R. and D. G. Shand, A physiological approach to hepatic drug clearance. Clinical Pharmacology & Therapeutics, 1975. 18(4): p. 377-390.
29. Shand, D. G., R. H. Cotham, and G. R. Wilkinson, Perfusion-limited effects of plasma drug binding on hepatic drug extraction. Life Sciences, 1976. 19(1): p. 125-130.
30. Tsao, S. C., et al., Kinetic analysis of albumin-mediated uptake of warfarin by perfused rat liver. Journal of Pharmacokinetics and Biopharmaceutics, 1988. 16(2): p. 165-181.
31. Miyauchi, S., et al., The Phenomenon of Albumin-Mediated Hepatic Uptake of Organic Anion Transport Polypeptide Substrates: Prediction of the In Vivo Uptake Clearance from the In Vitro Uptake by Isolated Hepatocytes Using a Facilitated-Dissociation Model. Drug Metabolism and Disposition, 2018. 46(3): p. 259.
32. Savic, R. M., et al., Implementation of a transit compartment model for describing drug absorption in pharmacokinetic studies. Journal of Pharmacokinetics and Pharmacodynamics, 2007.34(5): p. 711-726.
33. Wilkins, J. J., et al., Population pharmacokinetics of rifampin in pulmonary tuberculosis patients, including a semimechanistic model to describe variable absorption. Antimicrobial Agents and Chemotherapy, 2008. 52(6): p. 2138-2148.
34. Everson, G. T., et al., The spectrum of hepatic functional impairment in compensated chronic hepatitis C: results from the Hepatitis C Anti-viral Long-term Treatment against Cirrhosis Trial. Alimentary Pharmacology & Therapeutics, 2008. 27(9): p. 798-809.
35. Helmke, S. M., et al., A disease severity index based on dual cholate clearances and shunt outperforms biopsy at predicting clinical outcomes in chronic Hepatitis C. Gastroenterology, 2013. 144(5, Supplement 1): p. S-951-S-952.
36. Helmke, S., et al., Significant alteration of the portal circulation in over half of the chronic HCV patients with Ishak fibrosis stage F0-F2. Hepatology, 2011. 54(4).
37. Burton, J. R., et al., The within-individual reproducibility of the disease severity index from the HepQuant SHUNT test of liver function and physiology. Translational Research, 2021.233: p. 5-15.
38. Koo, T. K. and M. Y. Li, A Guideline of Selecting and Reporting Intraclass Correlation Coefficients for Reliability Research. Journal of Chiropractic Medicine, 2016. 15(2): p. 155-163.
39. McGraw, K. O. and S. P. Wong, Forming inferences about some intraclass correlation coefficients. Psychological Methods, 1996. 1(1): p. 30-46.
40. Combleet, P. J. and N. Gochman, Incorrect least-squares regression coefficients in method-comparison analysis. Clinical Chemistry, 1979. 25(3): p. 432-8.
41. Lawitz, E., et al., BI 685509 improves hepatic function in subjects with Child-Pugh A cirrhosis and a liver stiffness measurement of >15 kPa: Results from the HepQuant SHUNT test. Hepatology, 2021(74): p. 1238A-1239A.
42. Derbyshire, E. R. and M. A. Marietta, Structure and Regulation of Soluble Guanylate Cyclase. Annual Review of Biochemistry, 2012. 81(1): p. 533-559.
43. Budworth, J., et al., Tissue Distribution of the Human Soluble Guanylate Cyclases. Biochemical and Biophysical Research Communications, 1999. 263(3): p. 696-701.
44. Zhao, Y., et al., A molecular basis for nitric oxide sensing by soluble guanylate cyclase. Proceedings of the National Academy of Sciences, 1999, 96(26): p. 14753-14758.
45. Wiest, R. and R. J. Groszmann, The paradox of nitric oxide in cirrhosis and portal hypertension: Too much, not enough. Hepatology, 2002. 35(2): p. 478-491.
46. Sarela, A. I., et al., Hepatic and splanchnic nitric oxide activity in patients with cirrhosis. Gut, 1999. 44(5): p. 749.
47. Morales-Ruiz, M., et al., Pathophysiology of Portal Hypertension. PanVascular Medicine, 2015: p. 3631-3665.
48. Langer, D. A. and V. H. Shah, Nitric oxide and portal hypertension: Interface of vasoreactivity and angiogenesis. Journal of Hepatology, 2006. 44(1): p. 209-216.

1. A method for assessing liver function in a subject having or suspected of having or contracting a liver disease, comprising
obtaining blood or serum sample concentration data of an orally administered distinguishable cholate compound collected from a subject at two time points after oral administration;
measuring the area under the curve of the blood or serum concentrations of the orally administered distinguishable cholate compound (AUCoral) in the subject comprising
simulating a full oral clearance curve using a compartmental model of oral cholate clearance, the compartmental model comprising the oral distinguishable cholate compound concentration data at the two time points, body mass index (BMI), body weight (BW), and optionally hematocrit (Hct) input values in the subject, and
calculating the area comprising trapezoidal numerical integration to obtain the AUCoral; and
calculating one or more indices of hepatic disease in the subject using the AUCoral, wherein the one or more indices is associated with liver function in the subject.

2. The method of claim 1, wherein the obtaining concentration data of the orally administered distinguishable cholate compound at the two time points comprises
receiving first and second blood or serum samples that had been collected from the subject at first and second time points following a single oral dose of a first distinguishable cholate compound; and
analyzing the samples to obtain the oral concentration data at the first and second time points, optionally
wherein the blood or serum samples had been collected within about 180 minutes, 120 minutes, 90 minutes, within about 75 minutes, or within about 60 minutes after the oral administration.

3. The method of claim 2, wherein the first and second blood or serum samples had been collected from the subject between at least about 5 min to about 90 min, 10 min to 75 min, 20 min to 60 min, 25 min to 55 min, 30 min to 50 min, 35 to 45 min, or about 40 min apart.

4. The method of claim 3, wherein the first and second blood or serum samples had been collected from the subject at about 20 min and about 60 min following the oral administration, respectively.

5. The method of claim 1, wherein the obtaining concentration data of the orally administered distinguishable cholate compound at the two time points comprises
receiving a single blood or serum sample that had been collected from the subject following administration of a first oral dose of a first distinguishable cholate compound and a second oral dose of a second distinguishable cholate compound to the subject; and
analyzing the single sample to obtain the oral concentration data of the first distinguishable cholate compound and second distinguishable cholate compound at the two time points, optionally
wherein the single blood or serum sample had been collected from the subject within about 180 minutes, 120 minutes, 90 minutes, within about 75 minutes, or within about 60 minutes after the first oral dose administration.

6. The method of claim 5, wherein the first and second oral doses had been administered to the subject between at least about 5 min to about 90 min, 10 min to 75 min, 20 min to 60 min, 25 min to 55 min, 30 min to 50 min, 35 to 45 min, or about 40 min apart.

7. The method of claim 5, wherein the single sample had been collected from the subject at about 20 min after the second oral dose and simultaneously at about 60 min after the first oral dose.

8. The method of claim 1, further comprising
estimating an area under the curve of blood or serum concentrations of an intravenously administered distinguishable cholate compound (AUCiv); and
calculating the one or more indices of hepatic disease in the subject using the AUCoral and AUCiv values.

9. The method of claim 8, wherein the estimating the AUCiv comprises a linear regression model, optionally wherein the linear regression model comprises equation 11A:
AUC_{IV}=β₀+β_{BW}·BW+β_{PO,20}·C_{PO,20}+β_{PO,60}·C_{PO,60}+β_{HFR,P}·HFR_{P} Eqn. 11A,
wherein
β₀ is an intercept coefficient, optionally wherein the intercept coefficient is 161.972;
β_{BW} is a body weight coefficient, optionally wherein the body weight coefficient is 0.6459;
BW is the subject's body weight in kg;
β_{PO,20} is an orally administered distinguishable cholate concentration coefficient at a first time point, optionally wherein the β_{PO,20} is 16.9249;
C_{PO,20} is the orally administered distinguishable cholate concentration at the first time point;
β_{PO,60} is an orally administered distinguishable cholate concentration coefficient at a second time point, optionally wherein the β_{PO,60} is 89.2405;
C_{PO,60} is an orally administered distinguishable cholate concentration at the second time point;
β_{HFR,P} is a portal HFR coefficient, optionally wherein the portal HFR coefficient is −0.4755; and
HFR_{P} is the subject's measured portal HFR.

10. The method of claim 8, wherein the estimating the AUCiv comprises
obtaining blood or serum sample concentration data of an intravenously administered third distinguishable cholate compound in one of the samples that had been collected from the subject, and
exponential fitting the intravenous concentration data to a systemic cholate clearance curve comprising fast, moderate, and slow phases of clearance over at least about 180 min after the iv administration of the intravenous dose.

11. The method of claim 10, wherein the third distinguishable cholate had been intravenously administered simultaneously with the first oral dose, or at least about 5 min to about 75 min, 10 min to 70 min, 20 min to 60 min, 25 min to 55 min, 30 min to 50 min, 35 to 45 min, or about 40 min after the first oral dose.

12. The method of claim 10, wherein the blood or serum sample collected at one time point had been collected within about 90 minutes or less, 75 minutes or less, 60 minutes or less, 45 minutes or less, 30 minutes or less, or about 20 minutes after the intravenous administration.

13. The method of claim 10, wherein the fitting to systemic cholate clearance curve fast phase (Y₀) is calculated according to equation 20:
Y₀=C₀·e^{-k}^{_{fast}}^{·t} Eqn. 20,
wherein
t=time (0 to 20 min);
C₀ is the initial concentration of intravenously administered distinguishable cholate compound,
C₂₀ is the measured 20-minute concentration of intravenously administered distinguishable cholate compound; and
k_{fast} is the rate of elimination in the fast phase estimated by equation 18:
$${k}_{fast} = \frac{ln ( {C}_{0} ) - ln ( {C}_{20} )}{20} , \qquad Eqn . 18$$
optionally wherein
C₀ is estimated according to equation 17;
$${C}_{0} = \frac{{D}_{IV}}{{V}_{d} \cdot BW} , \qquad Eqn . 17$$
wherein
D_{IV} is the intravenous dose of third distinguishable cholate;
BW is subject body weight (kg); and
Vd is the volume of distribution (V_{d}) in L per kg body weight, calculated according to equation 16A:
$$TPV = {V}_{d} = \frac{0.07}{\sqrt{BMI / 22}} \cdot ( 1 - Hct ) , \qquad Eqn . 16 A$$
wherein TPV is total plasma volume, BMI is body mass index, and Hct is hematocrit in the subject.

14. The method of claim 10, wherein the fitting to systemic cholate clearance curve moderate phase (Y₁) is calculated according to equation 21:
$${Y}_{1} = \frac{{C}_{20}}{{e}^{- {k}_{mod} \cdot 20}} \cdot {e}^{- {k}_{mod} \cdot t} , \qquad Eqn . 21$$
wherein
t=time (20-45 min);
k_{mod} is the rate of elimination in the moderate phase estimated by equation 19:
k_{mod}=β_{0,mod}+β_{kfast}·k_{fast}+β_{IV,20}·C_{IV,20}+β_{PO,20,mod}·C_{PO,20}+β_{PO,60,mod}·C_{PO,60} Eqn. 19,
wherein
β_{0,mod} is the intercept coefficient, optionally wherein the intercept coefficient is 0.0268;
β_{kfast} is the k_{fast} coefficient, optionally wherein the k_{fast} coefficient is 0.546;
β_{IV,20} is the coefficient of the intravenous distinguishable cholate concentration at the 20 min time point; optionally wherein β_{IV,20} is 0.0045;
β_{PO,20,mod} is the coefficient of the oral distinguishable cholate concentration at the 20 min time point, optionally wherein β_{PO,20,mod} is −0.0007;
β_{PO,60,mod} is the coefficient of the oral distinguishable cholate concentration at the 60 min time point, optionally wherein β_{PO,60,mod} is −0.0052.

15. The method of claim 10, wherein the fitting to systemic cholate clearance curve slow phase (Y₂) is calculated according to equation 22:
$${Y}_{2} = \frac{{C}_{45}}{{e}^{- {k}_{slow} \cdot 45}} \cdot {e}^{- {k}_{slow} \cdot t} , \qquad Eqn . 22$$
wherein
t=time (45-180 min);
C₄₅ is the estimated 45-minute concentration of intravenously administered distinguishable cholate; and
k_{slow} is the rate of elimination in the slow phase estimated by a mean value from a multiplicity of CLD patients, optionally wherein k_{slow} is 0.018 min⁻¹.

16. The method according to claim 10, wherein the areas under each of the three exponential curve fits are calculated by trapezoidal numerical integration and summed to estimate the AUC_{IV}.

17. The method of claim 1, wherein the compartmental model of oral clearance comprises estimating
compartment volumes of a plurality of compartments in the subject; and
flow parameters between the plurality of compartments in the subject.

18. The method of claim 17, wherein the compartmental model further comprises estimating cholate binding and dose administration in the subject.

19. The method of claim 17, wherein the plurality of compartments in the subject comprises systemic, portal, and liver compartments.

20. The method of claim 18, wherein the estimating compartment volumes of the plurality of compartments comprises
estimating the systemic compartment volume (V_{S}), the portal compartment volume (V_{P}), and the liver compartment volume (V_{L}) in the subject, optionally each compartment volume in liters (L).

21. The method of claim 20, wherein the systemic compartment volume (V_{S}) is estimated according to Equation 4A:
V_{S}=TBV·(1−Hct) Eqn. 4A,
wherein
TBV is total blood volume in the subject according to equation 4:
$$TBV = \frac{0.07 \cdot BW}{\sqrt{BMI / 22}} , \qquad Eqn . 4$$
wherein
BMI is body mass index (kg/m²) in the subject, and
BW is body weight (kg) in the subject; and
Hct is the hematocrit in the subject.

22. The method of claim 20, wherein the portal compartment volume (V_{P}) is estimated according to Equation 4B:
V_{P}=0.25·V_{S} Eqn. 4B.

23. The method of claim 20, wherein the liver compartment volume (V_{L}) is estimated according to Equation 5A:
V_{L}=(0.275·22.46·BW·d_{L}·fₚₗₐₛₘₐ)/1000 Eqn. 5A,
wherein d_{L} is assumed Liver tissue density of 1.06 g·mL⁻¹.

24. The method of claim 17, wherein the estimating the flow parameters between the plurality of compartments comprises a system of first-order ordinary differential equations 1A, 2A, and 3:
$$\frac{{dC}_{S}}{dt} = \frac{1}{{V}_{S}} ( - [ {q}_{SP} + {q}_{SL} ] {C}_{S} + {q}_{LS} \cdot {C}_{L} ) ; \qquad Eqn . 1 A$$
$$\frac{{dC}_{P}}{dt} = \frac{1}{{V}_{P}} ( {D}_{PO} + {q}_{SP} \cdot {C}_{S} - {q}_{PL} \cdot {C}_{P} ) ; and \qquad Eqn . 2 A$$
$$\frac{{dC}_{L}}{dt} = \frac{1}{{V}_{L}} ( - [ {Cl}_{H} \cdot {V}_{L} + {q}_{LS} ] {C}_{L} + {q}_{PL} \cdot {C}_{P} + {q}_{SL} \cdot {C}_{S} ) , \qquad Eqn . 3$$
wherein V is the volume of each of the each of the systemic (V_{S}), portal (V_{P}), and liver (V_{L}) compartments, C is the concentration of orally administered distinguishable cholate compound in each of the systemic (C_{S}), portal (C_{P}), and liver (C_{L}) compartments, q is the flow rate between compartments, and Cl_{H} is the hepatic clearance, and D_{PO,rate} is the rate of orally administered distinguishable cholate compound entering the portal compartment.

25. The method of claim 17, wherein the estimating the flow parameters between the plurality of compartments comprises a system of first-order ordinary differential equations 1B, 2B, and 3:
$$\frac{{dC}_{S}}{dt} = \frac{1}{{V}_{S}} ( - [ {q}_{SP} + {q}_{SL} ] {C}_{S} + {q}_{LS} \cdot {C}_{L} + {q}_{PS} \cdot {C}_{P} ) ; \qquad Eqn . 1 B$$
$$\frac{{dC}_{P}}{dt} = \frac{1}{{V}_{P}} ( {D}_{PO , rate} + {q}_{SP} \cdot {C}_{S} - {q}_{PL} \cdot {C}_{P} - {q}_{PS} \cdot {C}_{P} ) ; and \qquad Eqn . 2 B$$
$$\frac{{dC}_{L}}{dt} = \frac{1}{{V}_{L}} ( - [ {Cl}_{H} \cdot {V}_{L} + {q}_{LS} ] {C}_{L} + {q}_{PL} \cdot {C}_{P} + {q}_{SL} \cdot {C}_{S} ) , \qquad Eqn . 3$$
wherein V is the volume of each of the each of the systemic (V_{S}), portal (V_{P}), and liver (V_{L}) compartments, C is the concentration of orally administered distinguishable cholate compound in each of the systemic (C_{S}), portal (C_{P}), and liver (C_{L}) compartments, q is the flow rate between compartments, and Cl_{H} is the hepatic clearance, and D_{PO,rate} is the rate of orally administered distinguishable cholate compound entering the portal compartment.

26. The method of claim 24, wherein the estimating comprises estimating total hepatic inflow to the liver (Q_{L}), splanchnic arterial circulation (q_{SP}), hepatic portal venous inflow to the liver (q_{PL}), total hepatic venous return flow to systemic circulation (q_{LS}), and hepatic arterial inflow to the liver (q_{SL}) in the subject.

27. The method of claim 26, wherein the estimating total hepatic inflow to the liver (Q_{L}) comprising both hepatic arterial (q_{SL}) and portal venous (q_{PL}) inflows to the liver according to equation 6A:
Q_{L}=q_{SL}+q_{PL}, (L·min⁻¹) Eqn. 6A,
wherein
q_{SL} is rate of hepatic arterial inflow to the liver,
q_{SL}=0.25·Q_{L} (L·min⁻¹); and
q_{PL} is rate of portal venous inflow to the liver,
q_{PL}=q_{SP} (L·min⁻¹), wherein
q_{SP} is splanchnic arterial blood flow rate to abdominal intestinal organs,
q_{SP}=0.75·Q_{L,init} (L·min⁻¹), optionally wherein
the initial estimate for Q_{L} is about 1 L·min⁻¹·kg⁻¹ liver wet weight.

28. The method of claim 26, wherein the calculating total hepatic venous return flow rate to systemic circulation (q_{LS}) comprises: q_{LS}=q_{PL}+q_{SL}.

29. The method of claim 24, wherein the hepatic clearance (Cl_{H}) is estimated by equation 7A:
Cl_{H}=Q_{L}·ER Eqn. 7A,
wherein
ER is the extraction ratio in the subject and is assumed to be 0.7 for the distinguishable cholate compound.

30. A method for assessing liver function in a subject having or suspected of having or contracting a liver disease, comprising
obtaining input data derived from the subject comprising blood or serum sample concentration data of an orally administered distinguishable cholate compound collected from a subject at two time points within 180 minutes after oral administration, body mass index (BMI), estimated volume of distribution (Vd), and optionally hematocrit (Hct) in the subject;
fitting the input data to a trained function fitting neural network comprising a training algorithm to generate a multiplicity of output points for oral and intravenous distinguishable cholate clearance curves;
generating oral and IV distinguishable cholate clearance curves from the fitted data;
measuring AUC_{Oral} and AUC_{IV} values for the subject comprising trapezoidal numerical integration; and
calculating one or more indices of hepatic disease in the subject using the AUCoral and/or and AUC_{IV} values wherein the one or more indices is associated with liver function in the subject.

31. A method for assessing liver function in a subject having or suspected of having or contracting a liver disease, comprising
obtaining input data derived from the subject comprising (i) blood or serum sample concentration data of an orally administered first distinguishable cholate compound collected from a subject at two time points within 180 minutes after oral administration, (ii) blood or serum sample concentration data of an intravenously administered second distinguishable cholate compound collected from a subject at one time point within 180 minutes after intravenous administration, (iii) estimated volume of distribution (Vd) in the subject, and (iv) estimated initial intravenous distinguishable cholate concentration at 0 minutes of the intravenously administered second distinguishable cholate based on the Vd;
fitting the input data to a trained function fitting neural network comprising a training algorithm to generate a multiplicity of output points for oral and intravenous distinguishable cholate clearance curves;
generating oral and IV distinguishable cholate clearance curves from the fitted data;
measuring AUC_{Oral} and AUC_{IV} values for the subject comprising trapezoidal numerical integration; and
calculating one or more indices of hepatic disease in the subject using the AUCoral and/or and AUC_{IV} values wherein the one or more indices is associated with liver function in the subject.

32. The method of claim 30, wherein the estimated Vd (L per kg body weight) in the subject is estimated by equation 16A:
$$TPV = {V}_{d} = \frac{0.07}{\sqrt{BMI / 22}} \cdot ( 1 - Hct ) , \qquad Eqn . 16 A$$
wherein TPV is total plasma volume, BMI is body mass index, and Hct is hematocrit in the subject.

33. The method of claim 30, wherein the neural network is configured for regression tasks and comprises a 2-layer feedforward network comprising hidden layers and an output layer, optionally comprising a sigmoid transfer function in the hidden layers and a linear transfer function in the output layer.

34. The method of claim 30, wherein the neural network comprising a training algorithm had been trained on a training data set comprising a multiplicity of oral and intravenous distinguishable cholate clearance curves estimated by a non-compartmental minimal model (MM) from a combination of normal control subjects and chronic liver disease patients.

35. The method of claim 30, wherein the training algorithm is selected from the group consisting of a Levenberg-Marquardt backpropagation, Bayesian Regularization, BFGS Quasi-Newton, Resilient Backpropagation, Scaled Conjugate Gradient, Conjugate Gradient with Powell/Beale Restarts, Fletcher-Power Conjugate Gradient, Polak-Ribiére Conjugate Gradient, One Step Secant, Variable Learning Rate Gradient Descent, Gradient Descent with Momentum, and Gradient Descent training algorithm.

36. The method of claim 30, wherein the output points comprise 1 to 10 minute, 2 to 8 minute, or 5-minute increments from 0 to 180 minutes after the oral administration, resulting in 180 to 18, 60 to 23, or 37 timepoints for each of the oral and IV clearance curves.

37. The method of claim 31, wherein the initial intravenous distinguishable cholate compound concentration at 0 minutes is estimated comprising
estimating Vd (L per kg body weight) in the subject by equation 16A:
$$TPV = {V}_{d} = \frac{0.07}{\sqrt{BMI / 22}} \cdot ( 1 - Hct ) ; \qquad Eqn . 16 A$$
and
dividing the IV dose by V_{d} times body weight (BW).

38. A method for assessing liver function in a subject having or suspected of having or contracting a liver disease, comprising:
(a) receiving a plurality of blood or serum samples collected from the subject following oral administration of a dose of a first distinguishable cholate compound (doseₒᵣₐₗ) to the subject and simultaneous intravenous co-administration of a dose of a second distinguishable cholate compound (doseᵢᵥ) to the subject, wherein the samples had been collected over no more than 180 minutes after administration;
(b) quantifying the concentration of the first and the second distinguishable cholate compounds; and
(c) generating individual subject oral and intravenous clearance curves from the concentration of the first and second distinguishable cholate compounds comprising using a computer algorithm curve fitting to model oral and intravenous clearance curves; and computing the area under the individualized oral and intravenous clearance curves (AUCoral) and (AUCiv), respectively, in the subject, wherein the multiplicity of samples comprise blood or serum samples collected from the subject at 5 time or more points, and
wherein the generating individual intravenous clearance curve comprises
estimating an initial intravenous distinguishable cholate compound concentration in the subject at 0 minutes comprising
estimating Vd (L per kg body weight) in the subject by equation 16A:
$$TPV = {V}_{d} = \frac{0.07}{\sqrt{BMI / 22}} \cdot ( 1 - Hct ) ; \qquad Eqn . 16 A$$
dividing the IV dose by V_{d} times body weight (BW) to obtain the initial estimated initial intravenous distinguishable cholate concentration; and
constructing the intravenous distinguishable cholate clearance curve comprising the estimated initial intravenous concentration.

39. The method of claim 1, wherein the one or more indices of hepatic disease is selected from the group consisting of portal hepatic filtration rate (HFRp), systemic hepatic filtration rate (HFRs), cholate SHUNT, liver disease severity index (DSI), indexed hepatic reserve (HR_{indexed}), and algebraic hepatic reserve (HR_{algebraic}), in the subject.

40. The method of claim 39, wherein the calculating one or more indices of hepatic disease comprises calculating portal hepatic filtration rate (HFRp) in the subject by equation 10A:
$${HFR}_{P} = \frac{{D}_{PO}}{{AUC}_{Oral} \cdot BW} , \qquad Eqn . 10 A$$
wherein D_{PO} is the oral dose of the orally administered distinguishable cholate, and BW is the subject body weight.

41. The method of claim 39, wherein the calculating one or more indices of hepatic disease comprises calculating systemic hepatic filtration rate (HFRs) according to equation 12:
$${HFR}_{S} = \frac{{D}_{IV}}{{AUC}_{IV} \cdot BW} , \qquad Eqn . 12$$
wherein D_{IV} is the administered intravenous oral dose of distinguishable cholate, and BW is the subject body weight.

42. The method of claim 8, wherein the calculating one or more indices of hepatic disease comprises estimating cholate SHUNT (F) according to equation 13:
$$F = \frac{{AUC}_{Oral} \cdot {D}_{IV}}{{AUC}_{IV} \cdot {D}_{PO}} . \qquad Eqn . 13$$

43. The method of claim 31, wherein the calculating one or more indices of hepatic disease comprises estimating liver Disease Severity Index (DSI) according to equation 14:
$$DSI = A \cdot \sqrt{{( ln [ \frac{{HFR}_{P , max}}{{HFR}_{P}} ] )}^{2} + {( ln [ \frac{{HFR}_{S , max}}{{HFR}_{S}} ] )}^{2}} , \qquad Eqn . 14$$
wherein
HFR_{P,max} is the upper limit of portal clearance from a multiplicity of healthy controls;
HFR_{S,max} is the upper limit of clearance from a multiplicity of healthy controls; and
A is a factor to scale DSI from 0 to 50.

44. The method of claim 40, wherein the calculating one or more indices of hepatic disease comprises estimating indexed Hepatic Reserve (HR_{indexed}) according to equation 15:
$${HR}_{indexed} = 100 - A \cdot \sqrt{{( ln [ \frac{{HFR}_{P , lean}}{{HFR}_{P}} ] )}^{2} + {( ln [ \frac{{HFR}_{S , lean}}{{HFR}_{S}} ] )}^{2}} , \qquad Eqn . 15$$
wherein
HFR_{P} and HFR_{S} are indexed to lean controls minus one standard deviation (HFR_{P,lean} and HFR_{S,lean}); and A is a constant to scale HR value from 100 to 0.

45. The method of claim 40, wherein the calculating one or more indices of hepatic disease comprises estimating algebraic Hepatic Reserve (HR_{algebraic}) according to equation 15A:
HR_{algebraic}=100−2·DSI Eqn. 15A.

46. The method of claim 10, wherein the first distinguishable cholate compound is a first stable isotope labeled cholate compound, the optional second distinguishable cholate compound is a second stable isotope labeled cholate compound, and the optional third distinguishable cholate compound is a third stable isotope labeled cholate compound, optionally wherein the first, optional second, and optional third stable isotope labeled cholate compounds are selected from d4-cholate, d5-cholate, d2 cholate, and ¹³C-cholate, optionally wherein the d4-cholate is 2,2,4,4-d4 cholate, the d5-cholate is 2,2,3,4,4-cholate, and further optionally wherein the 13C cholate is 24-¹³C-cholate.

47. (canceled)

48. (canceled)

49. The method of claim 1, further comprising
comparing the one or more indices of hepatic disease in the subject to one or more cutoff values as an indicator of the relative hepatic function in the subject, optionally wherein the one or more cutoff values are derived from one or more normal healthy controls, a group of known patients, or within the subject over time.

50. (canceled)

51. The method of claim 1, further comprising
providing the one or more indices of hepatic disease to a medical professional for the purpose of developing a treatment plan in the subject.

52. The method of claim 51, wherein the one or more indices of hepatic disease is employed for a purpose selected from the group consisting of determining a need for treatment, predicting response to treatment, monitoring the effectiveness of a treatment, predicting large esophageal varices, personalized dosing of a drug, and predicting risk of clinical outcome in the subject, optionally wherein the clinical outcome is selected from the group consisting of Child-Turcotte-Pugh (CTP) progression, Model for End-stage Liver Disease (MELD) progression, variceal hemorrhage, ascites, splenomegaly, varices, large esophageal varices, portal hypertension (PHTN), hepatic encephalopathy, hepatocellular carcinoma (HCC), decompensation, or liver-related death.

53. The method of claim 1, wherein the liver disease is a chronic liver disease selected from the group consisting of chronic hepatitis C (CHC), chronic hepatitis B, metabolic dysfunction-associated alcoholic liver disease (Met-ALD), alcoholic liver disease (ALD), steatotic liver disease (SLD), fatty liver disease, Alcoholic SteatoHepatitis (ASH), Alcoholic Hepatitis (AH), metabolic dysfunction-associated steatotic liver disease (MASLD), Non-Alcoholic Fatty Liver Disease (NAFLD), steatosis, metabolic dysfunction-associated steatohepatitis (MASH), Non-Alcoholic SteatoHepatitis (NASH), autoimmune liver disease, cryptogenic cirrhosis, hemochromatosis, Wilson's disease, alpha-1-antitrypsin deficiency, liver cancer, liver failure, cirrhosis, primary sclerosing cholangitis (PSC), and other cholestatic liver diseases.

54. (canceled)

55. (canceled)

56. The method of claim 52, wherein monitoring the need for treatment in the subject comprises
determining the one or more indices of hepatic disease in the subject; and
comparing the one or more indices of hepatic disease to one or more cutoff value(s),
wherein a change in the one or more indices of hepatic disease compared to cutoff value(s) is indicative of the need for treatment in the subject.

57. (canceled)

58. The method of claim 57, wherein the fibrosis stage is determined by a method comprising liver biopsy or elastography, optionally wherein the liver biopsy determines Ishak fibrosis score (liver biopsy) of F2 (mild portal fibrosis), F3, F4 (moderate bridging fibrosis), F5 (nodular formation and incomplete cirrhosis), or F6 (cirrhosis).

59. (canceled)

60. The method of claim 1, further comprising obtaining one or more laboratory values, subject characteristics, and/or clinical scores associated with liver function in the subject.

61. The method of claim 60, wherein the laboratory values are selected from the group consisting of platelet count, albumin, prothrombin time international normalized ratio (PT INR), alkaline phosphatase (alk phos), creatinine, alanine aminotransferase (ALT), aspartate aminotransferase (AST), bilirubin, gamma-glutamyl transferase (GGT), APRI (AST to platelet ratio index), FIB-4, triglyceride, ferritin, and glucose.

62. The method of claim 60, wherein the one or more subject characteristics are selected from the group consisting of BMI, obesity, gender, and age.

63. The method of claim 60, wherein the one or more clinical scores are selected from the group consisting of Child-Turcotte-Pugh (CTP) score, model for end-stage liver disease (MELD) score, Ishak fibrosis score, METAVIR fibrosis stage by liver biopsy, BRUNT/KLEINER score, NASH risk factors, and fibrosis stage.

64. The method of claim 63, wherein the one or more clinical scores comprise histological assessment comprising liver biopsy and/or elastography.
